(19) United States
(12) Patent Application Publication
(10) Pub. No.: US 2013/0072458 A1
(43) Pub. Date: Mar. 21, 2013

(54) Methods of Treating Viral Associated Diseases

(71) Applicants: George R. Painter, Chapel Hill, NC (US); Ernest Randall Lanier, Chapel Hill, NC (US); Gwendolyn Powell Painter, Chapel Hill, NC (US)

(72) Inventors: George R. Painter, Chapel Hill, NC (US); Ernest Randall Lanier, Chapel Hill, NC (US); Gwendolyn Powell Painter, Chapel Hill, NC (US)

(73) Assignee: Chimerix, Inc., Durham, NC (US)

(21) Appl. No.: 13/504,785

(22) PCT Filed: Oct. 29, 2010
(86) PCT No.: PCT/US10/54779

§ 371 Date: Aug. 30, 2012

Related U.S. Application Data
(60) Provisional application No. 61/256,701, filed on Oct. 30, 2009.
(60) Provisional application No. 61/326,991, filed on Apr. 22, 2010.
(60) Provisional application No. 61/326,989, filed on Apr. 22, 2010.
(60) Provisional application No. 61/326,982, filed on Apr. 22, 2010.
(60) Provisional application No. 61/326,986, filed on Apr. 22, 2010.
(60) Provisional application No. 61/327,474, filed on Apr. 23, 2010.
(60) Provisional application No. 61/327,914, filed on Apr. 26, 2010.
(60) Provisional application No. 61/328,491, filed on Apr. 27, 2010.
(60) Provisional application No. 61/330,624, filed on May 3, 2010.
(60) Provisional application No. 61/331,704, filed on May 5, 2010.
(60) Provisional application No. 61/355,430, filed on Jun. 16, 2010.
(60) Provisional application No. 61/405,073, filed on Oct. 20, 2010.
(60) Provisional application No. 61/405,080, filed on Oct. 20, 2010.
(60) Provisional application No. 61/405,075, filed on Oct. 20, 2010.
(60) Provisional application No. 61/405,084, filed on Oct. 20, 2010.

(51) Int. Cl. A61K 31/675

(52) U.S. Cl. CPC A61K 31/675
USPC 514/81

(57) ABSTRACT

The present invention provides methods of treating diseases associated with at least one virus. The methods include administering a compound described in the invention in a therapeutically effective amount. According to some aspects of the present invention, the methods may further comprise at least one immunosuppressant agent to treat diseases associated with at least one virus of a subject in need of immunosuppressant agents.

### RELATED APPLICATIONS

This application claims the benefit under 35 U.S.C. §119(e) of U.S. Provisional Patent Application Ser. No. 61/256,701, filed Oct. 30, 2009; U.S. Provisional Patent Application Ser. No. 61/326,991, filed Apr. 22, 2010; U.S. Provisional Patent Application Ser. No. 61/326,989, filed Apr. 22, 2010; U.S. Provisional Patent Application Ser. No. 61/326,982, filed Apr. 22, 2010; U.S. Provisional Patent Application Ser. No. 61/326,986, filed Apr. 22, 2010; U.S. Provisional Patent Application Ser. No. 61/327,474, filed Apr. 23, 2010; U.S. Provisional Patent Application Ser. No. 61/327,914, filed Apr. 26, 2010; U.S. Provisional Patent Application Ser. No. 61/328,491, filed Apr. 27, 2010; U.S. Provisional Patent Application Ser. No. 61/330,624, filed May 3, 2010; U.S. Provisional Patent Application Ser. No. 61/331,704, filed May 5, 2010; U.S. Provisional Patent Application Ser. No. 61/355,430, filed Jun. 16, 2010; U.S. Provisional Patent Application Ser. No. 61/405,073, filed Oct. 20, 2010; U.S. Provisional Patent Application Ser. No. 61/405,080, filed Oct. 20, 2010; U.S. Provisional Patent Application Ser. No. 61/405,075, filed Oct. 20, 2010 and U.S. Provisional Patent Application Ser. No. 61/405,084, filed Oct. 20, 2010, the disclosures of which are incorporated herein by reference in their entireties.

### FIELD OF THE INVENTION

The present invention concerns methods of treating diseases associated with at least one virus with nucleoside phosphonates, in particular diseases associated with polyomavirus.

### BACKGROUND OF THE INVENTION

BK and JC viruses are polyomaviruses that infect more than two thirds of the healthy adult population without obvious clinical symptoms. BK virus is transmitted during childhood and known to persist in a state of latent infection in the renourinary tract with intermitted periods of asymptomatic shedding into urine (See Egli A, et al. (2009) Prevalence of polyomavirus BK and JC infection and replication in 400 healthy blood donors, J Infect Dis 199 (6); 837-846; Hirsch, H H, et al. (2003), Polyomavirus BK, Lancet Infect. Dis. 3, 611-623). In contrast to BK virus, JC virus seroprevalence follows later and continues to increase during adult life. Although infection with these viruses is generally asymptomatic in healthy individuals, patients with immunodeficiency, such as transplant patients, are at risk. BK virus diseases include polyomavirus-associated nephropathy (PVAN) affecting 1-10% of kidney transplant patients and polyomavirus-associated hemorrhagic cystitis (PVHC) affecting 5-15% of patients after allogenic hematopoietic stem cell transplantation (See Hirsch, H H (2005), BK virus: opportunity makes a pathogen. Clin. Infect. Dis. 41, 354-360.). The key disease caused by JC virus is polyomavirus-associated multifocal leukoencephalopathy (PVML)(See Padgett B L, et al., (1971) Cultivation of papova-like virus from human brain with progressive multifocal leucoencephalopathy, Lancet. June 19; 1 (7712);1257-60), and less frequently polyomavirus-associated nephropathy (See Drachenberg C B, et al. (2007) Polyomavirus BK versus JC replication and nephropathy in renal transplant recipients: a prospective evaluation. Transplantation. 84:323-30). Currently, there are no antiviral drugs with proven efficacy for polyomavirus-associated nephropathy or hemorrhagic cystitis in kidney- and bone marrow transplant patients, respectively.

### SUMMARY OF THE INVENTION

A first aspect of the invention is methods of treating conditions/disease associated with at least one virus in a subject. The method comprises administering to the subject a therapeutically effective amount of compounds described herein. The compounds described herein are specifically targeted against viral replication and/or virally infected/transformed cells. In one embodiment, the subject is immunocompromised.

In some embodiments, the disease associated with the virus is selected from nephropathy, hemorrhagic cystitis, or progressive multifocal leukoencephalopathy (PML). In another embodiment, nephropathy or hemorrhagic cystitis is associated with at least one polyomavirus (e.g., BK virus or JC virus). Further, in one embodiment, hemorrhagic cystitis is associated with at least one adenovirus (e.g., serotypes 11 and 12 of subgroup B). In one embodiment, the progressive multifocal leukoencephalopathy (PML) is associated with at least one JC virus.

In some embodiments, the disease is associated with at least one virus selected from polyomavirus (including BK, John Cunningham virus (JCV), Merkel cell virus (MCV), KI polyomavirus (KIV), WU polyomavirus (WUV), Simian virus 40 (SV 40)), papillomavirus (including human papillomavirus, cottontail rabbit papillomavirus, equine papillomavirus and bovine papillomavirus), herpes virus (e.g., herpes simplex virus), adenovirus, Epstein-Barr virus (EBV), human cytogegalovirus (HCMV), Hepatitis B virus, Hepatitis C virus, varicella zoster virus (VZV) or a combination thereof.

In one embodiment, the compound is

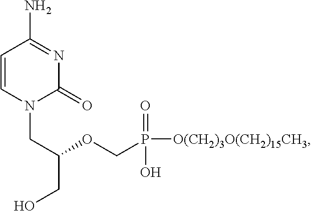

or a pharmaceutically acceptable salt thereof.

A further aspect of the invention provides methods for treating disease associated with at least one virus in a subject in need of an immunosuppressant agent. The methods include administering to the subject a therapeutically effective amount of compound described herein in combination with one or more immunosuppressant agents.

In some embodiments, at least one immunosuppressant agent is selected from Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm or a combination thereof.

In one embodiment, at least one immunosuppressant agents is Tysabri (natalizumab).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the effect of increasing concentrations of CNIX001 on BKV load and expression of BKV proteins. FIG. 1(a) illustrates the relationship between the concentration of CMX001 and reduction of extracellular BKV load. FIG. 1(b) shows the image of immunofluorescence staining 72 h p.i. of BKV-infected RPTECs.

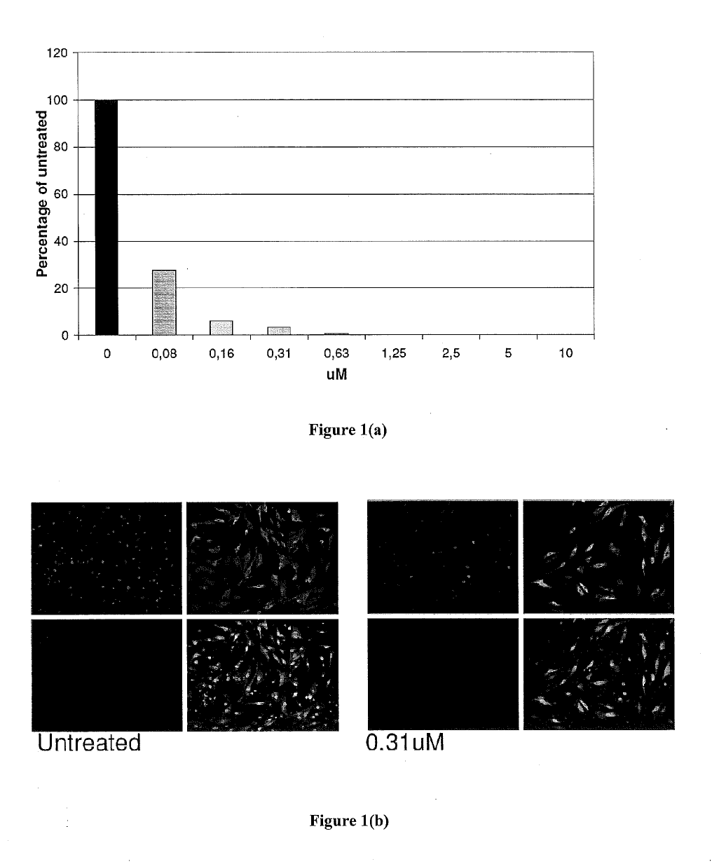

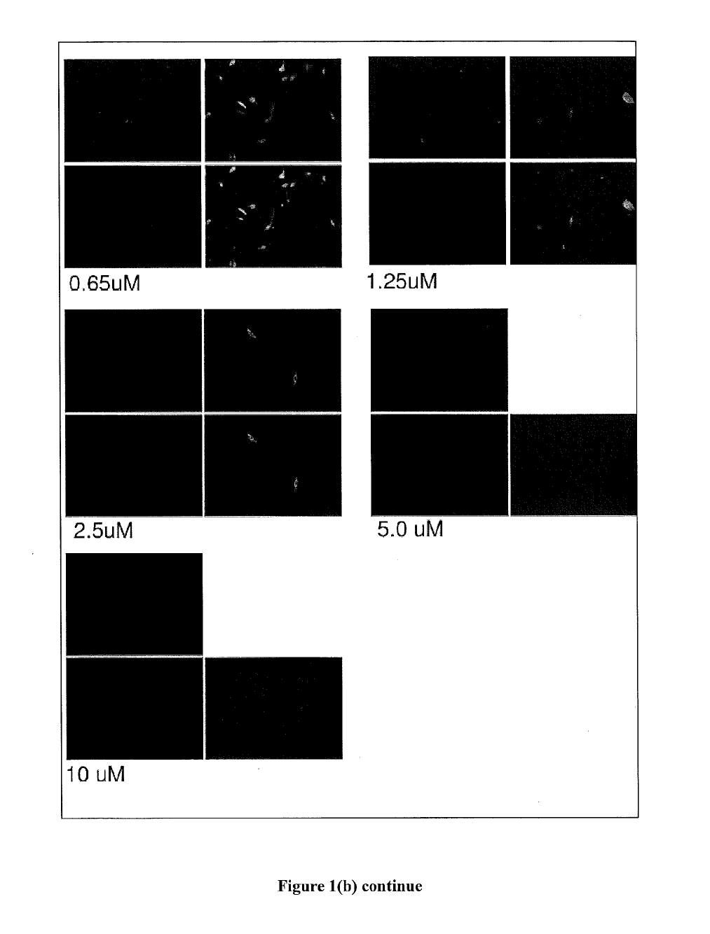

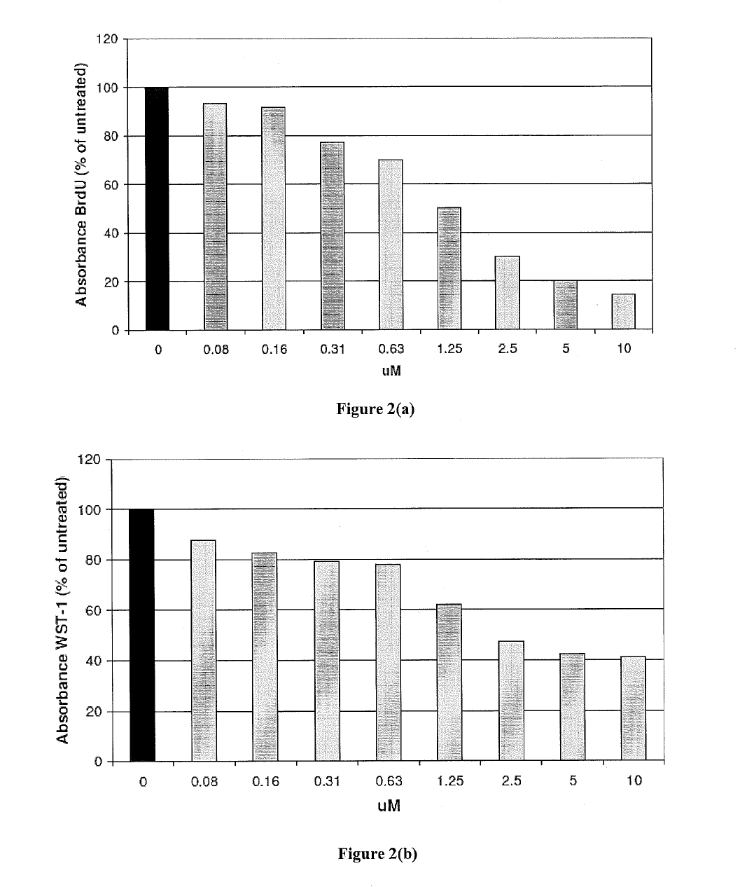

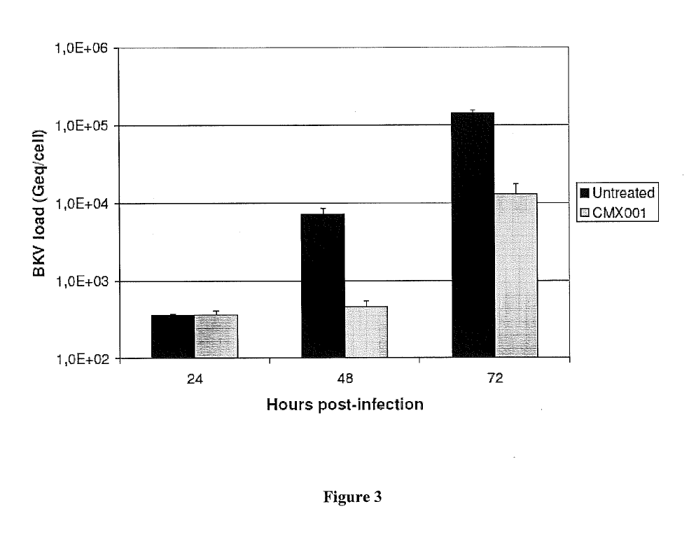

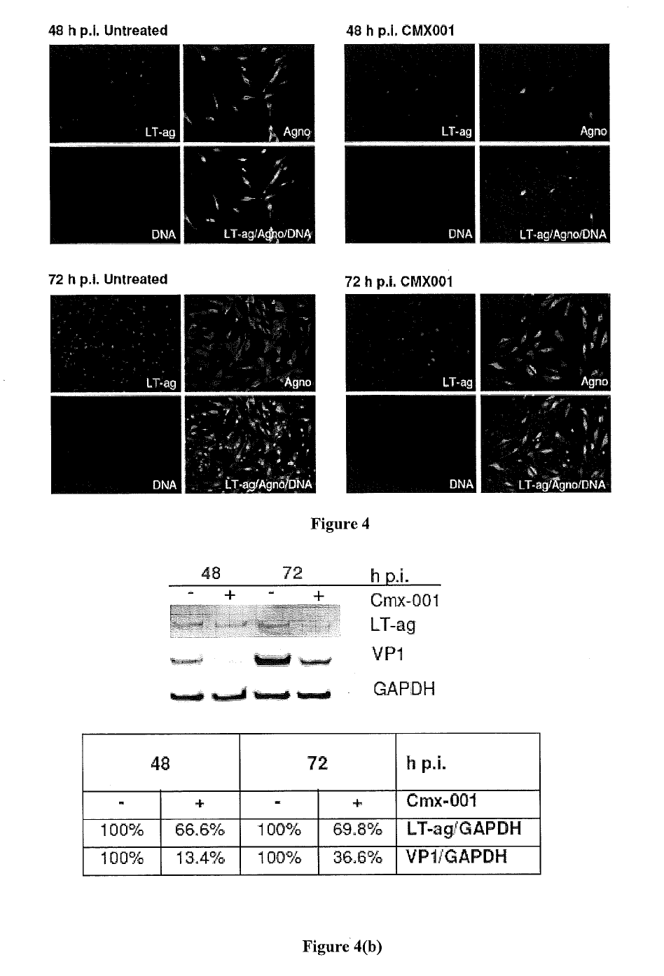

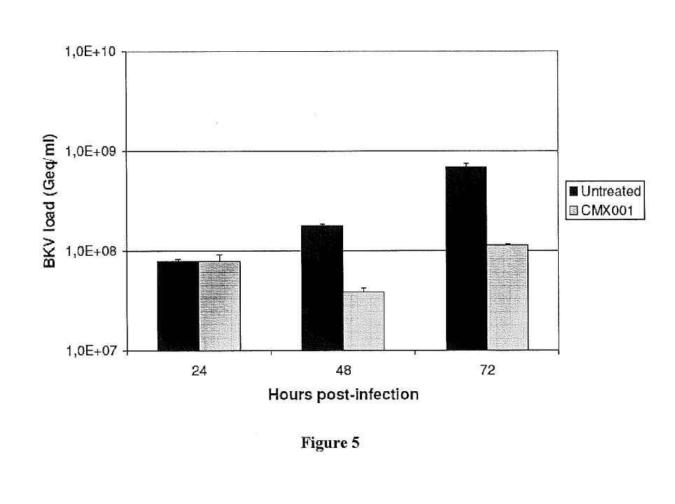

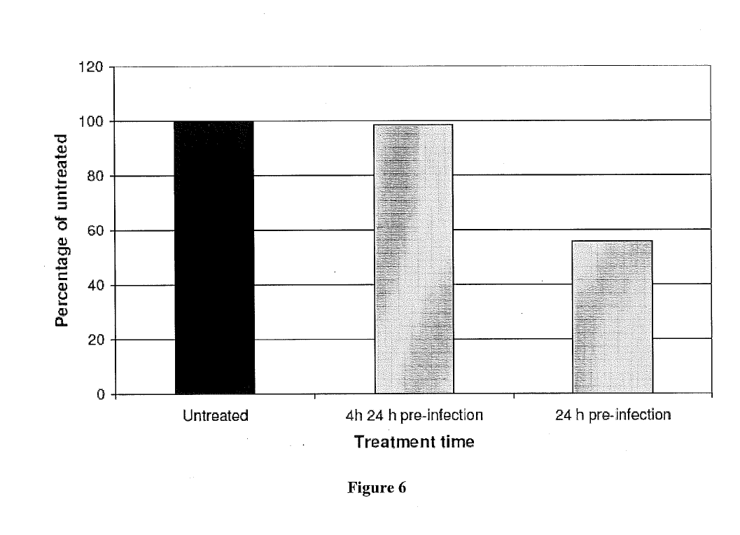

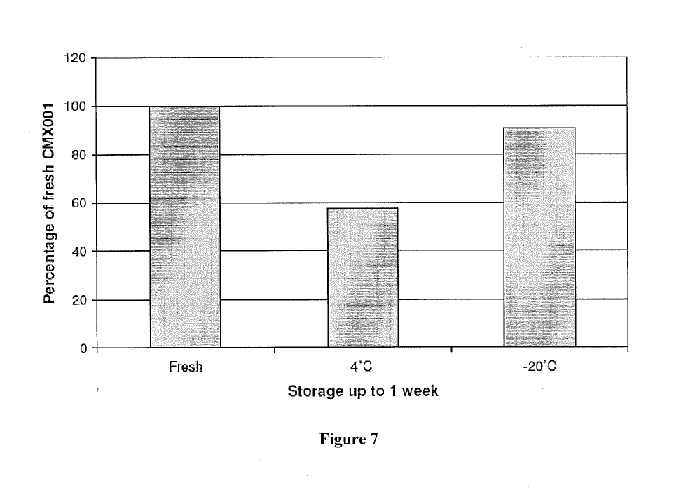

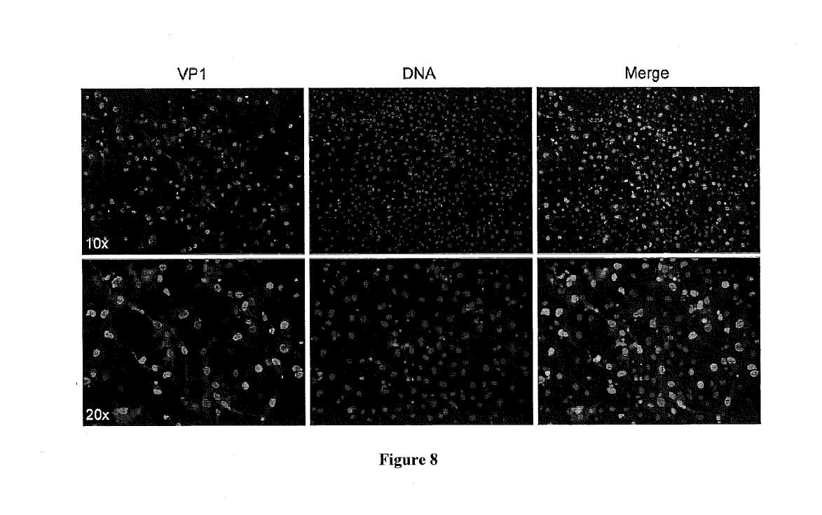

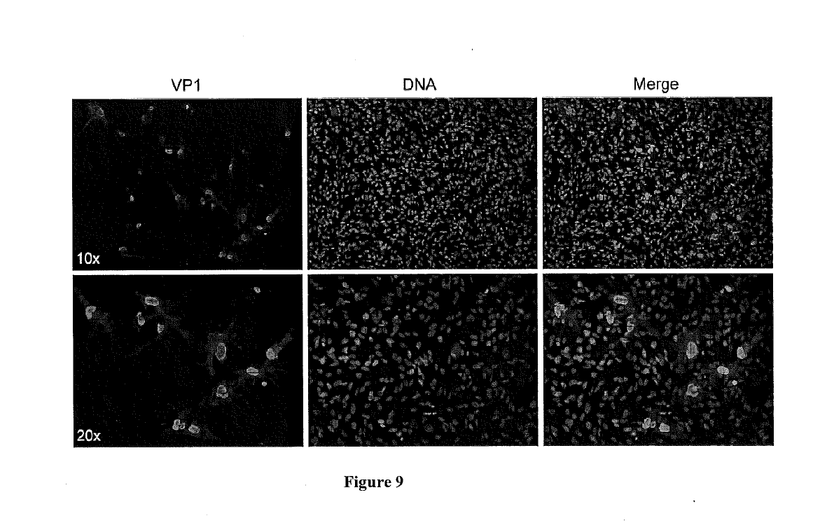

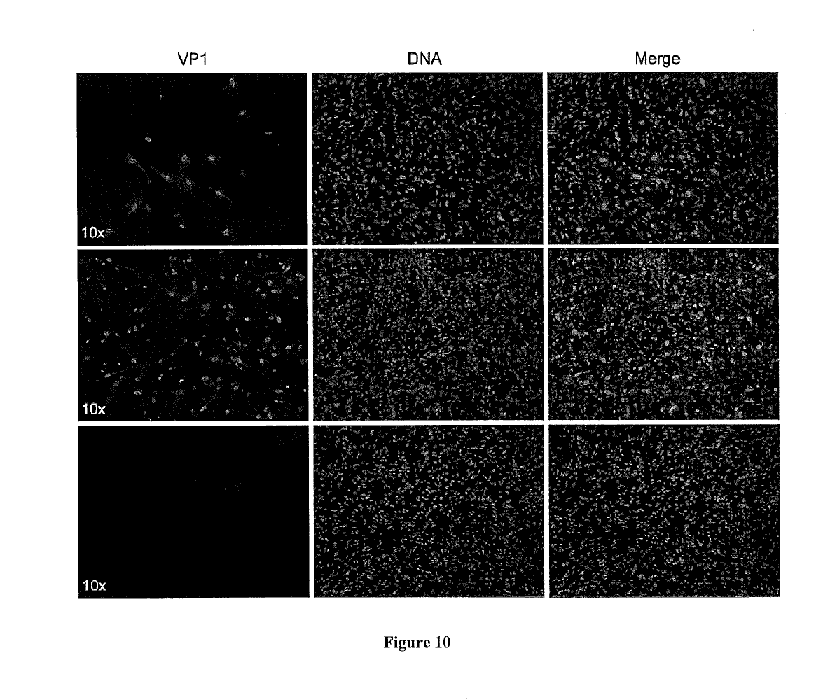

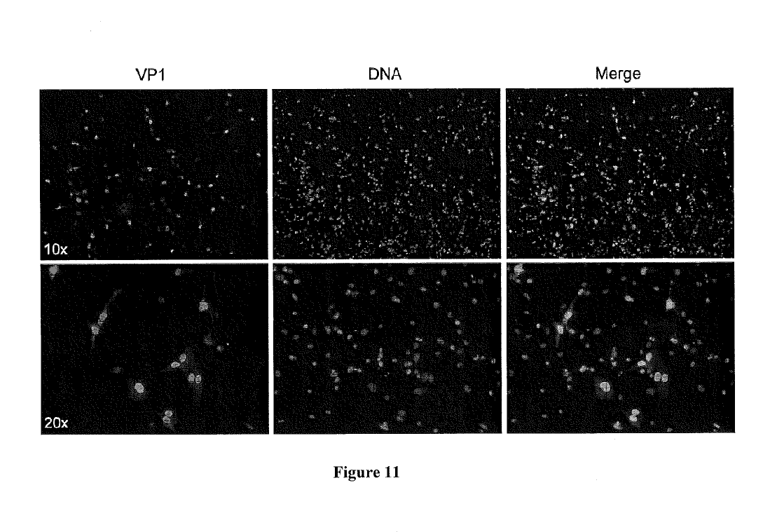

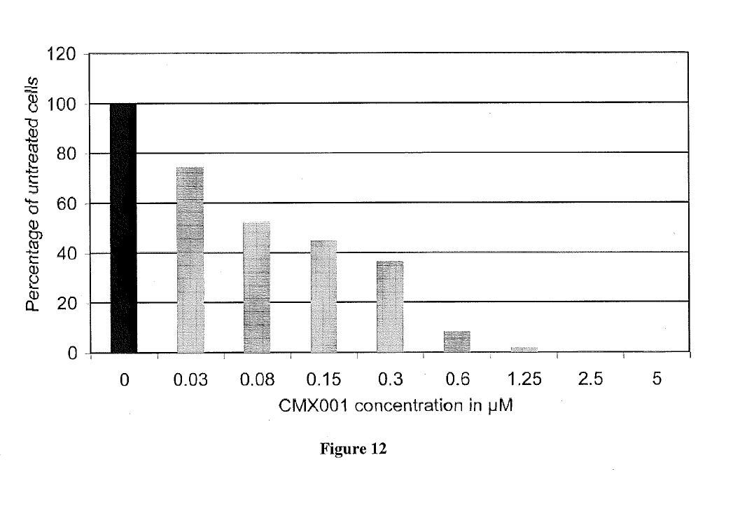

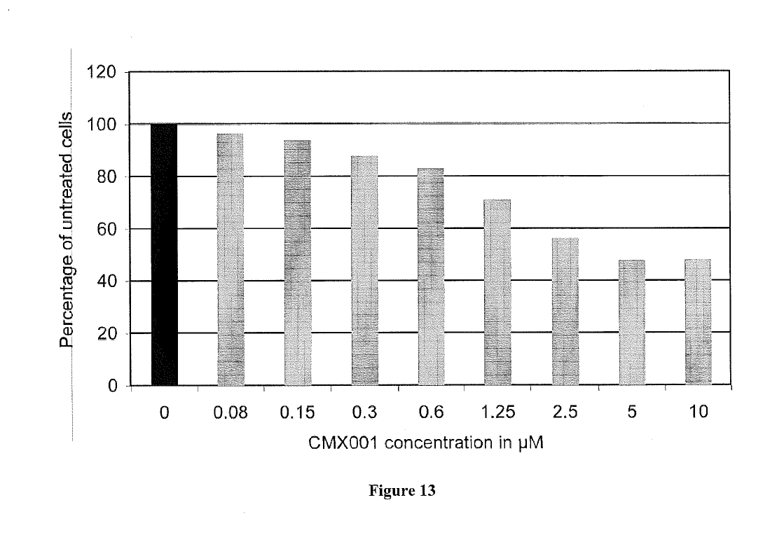

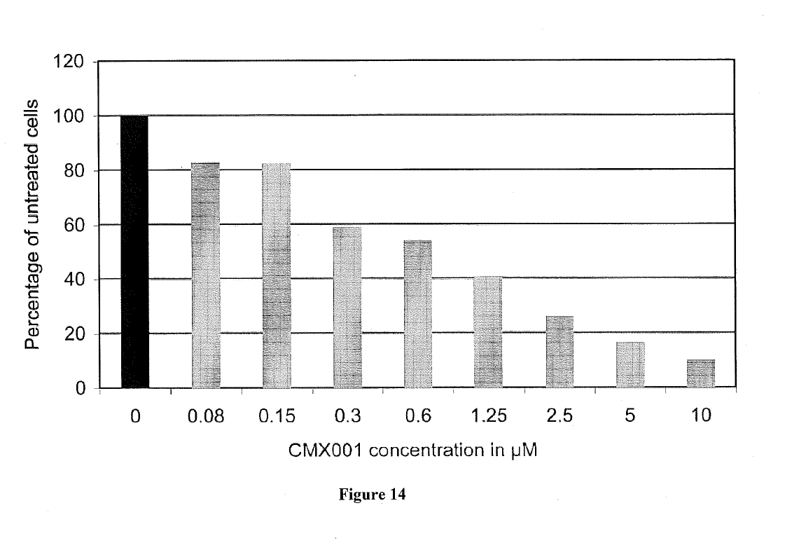

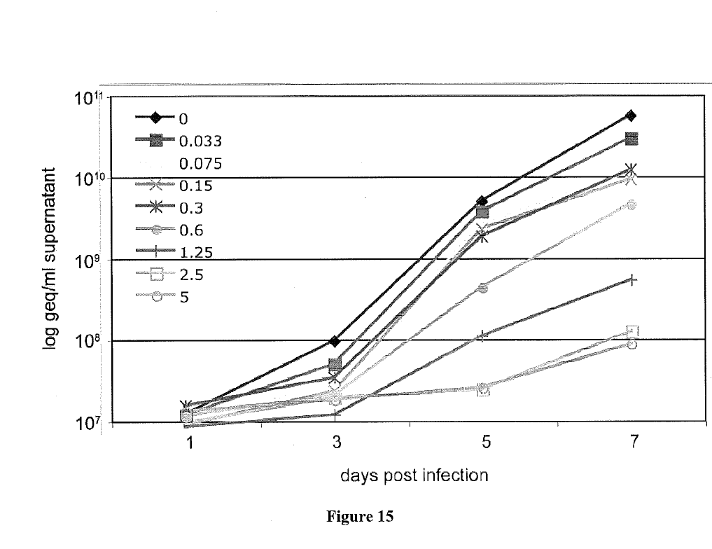

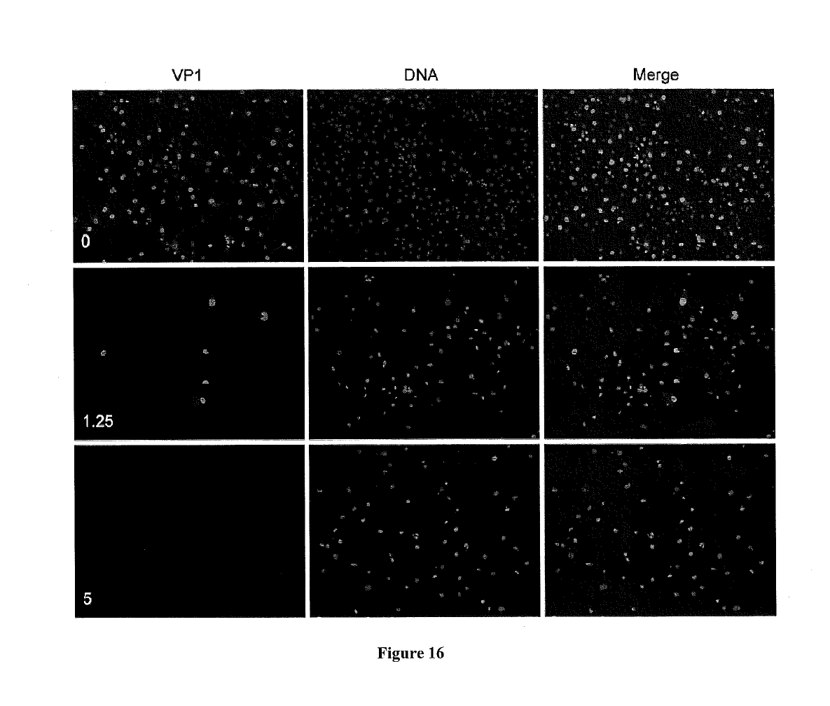

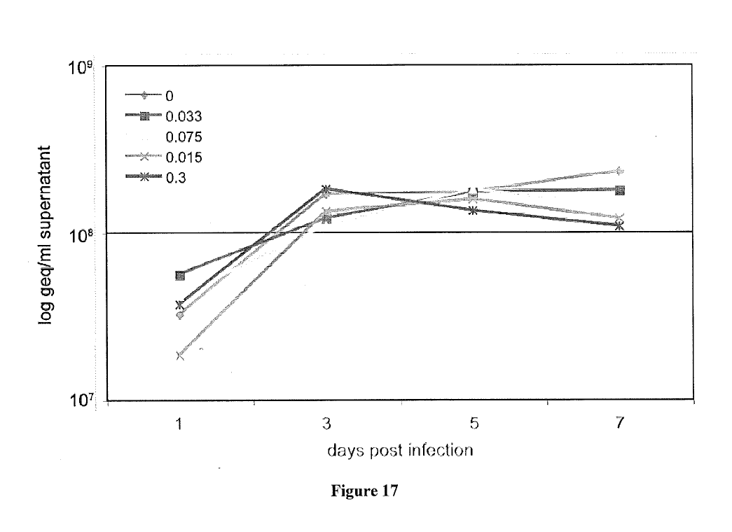

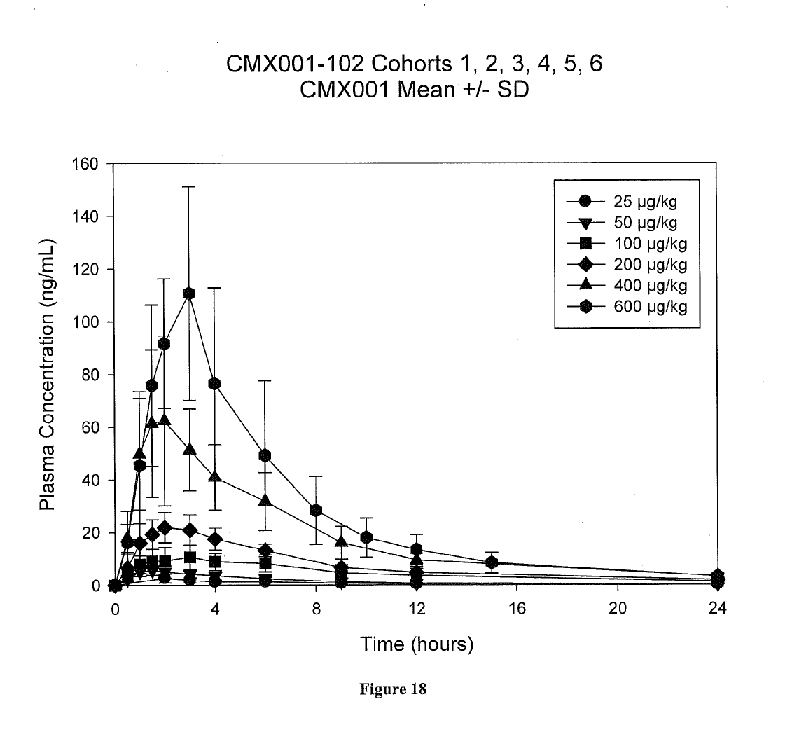

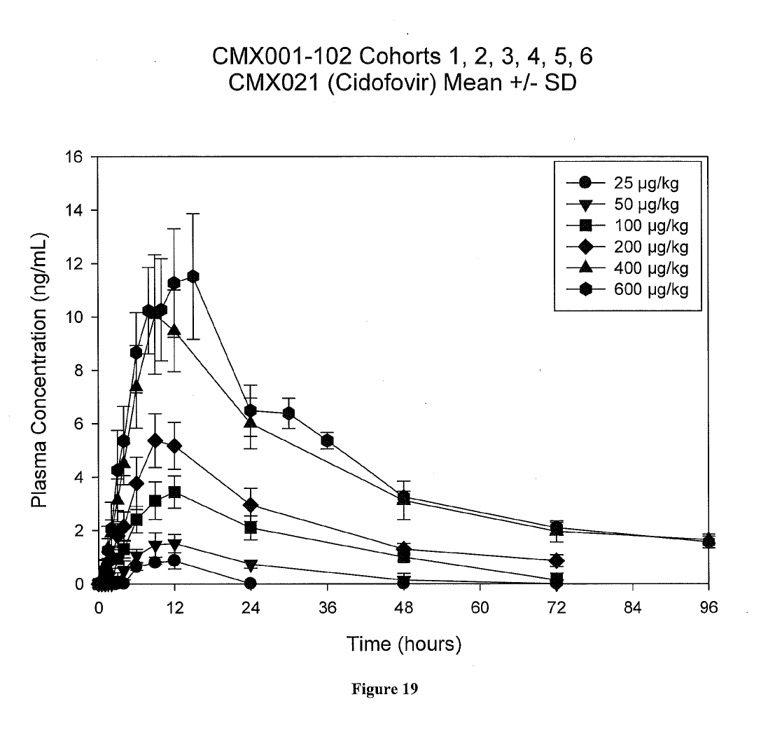

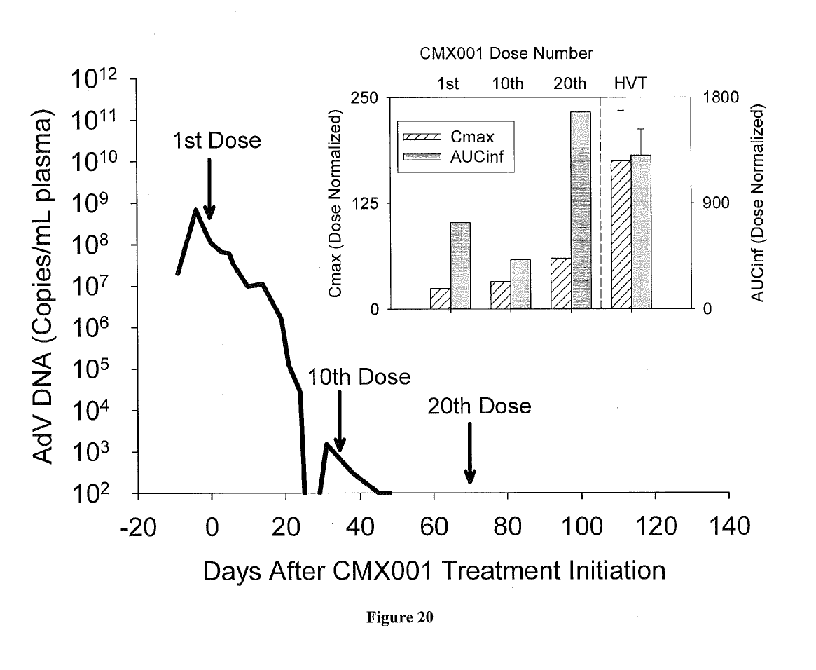

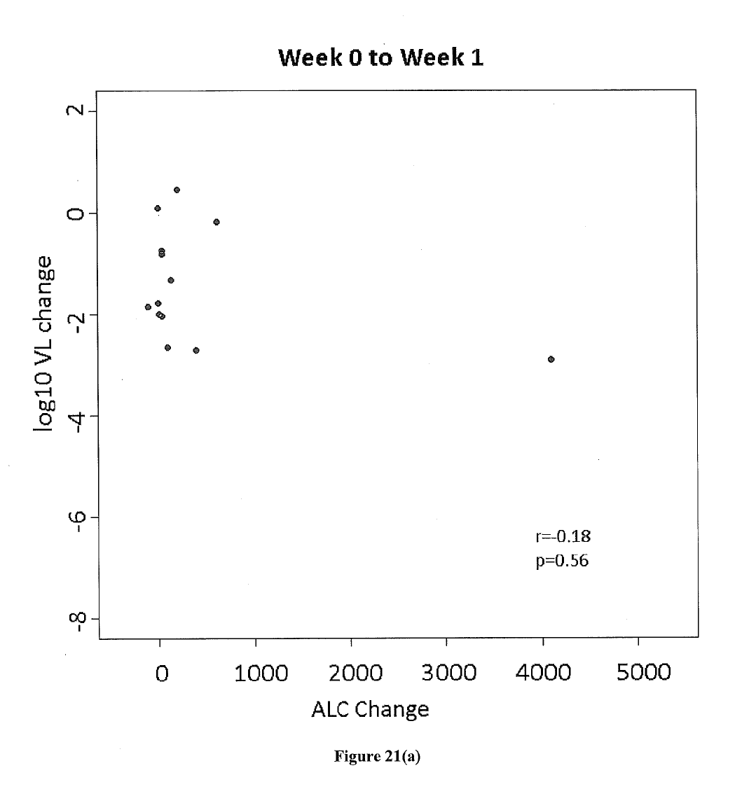

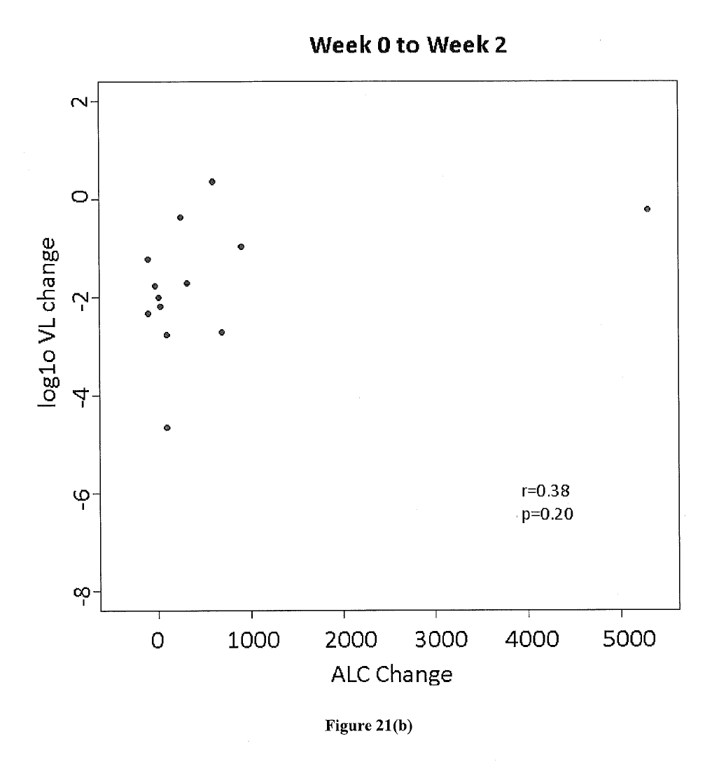

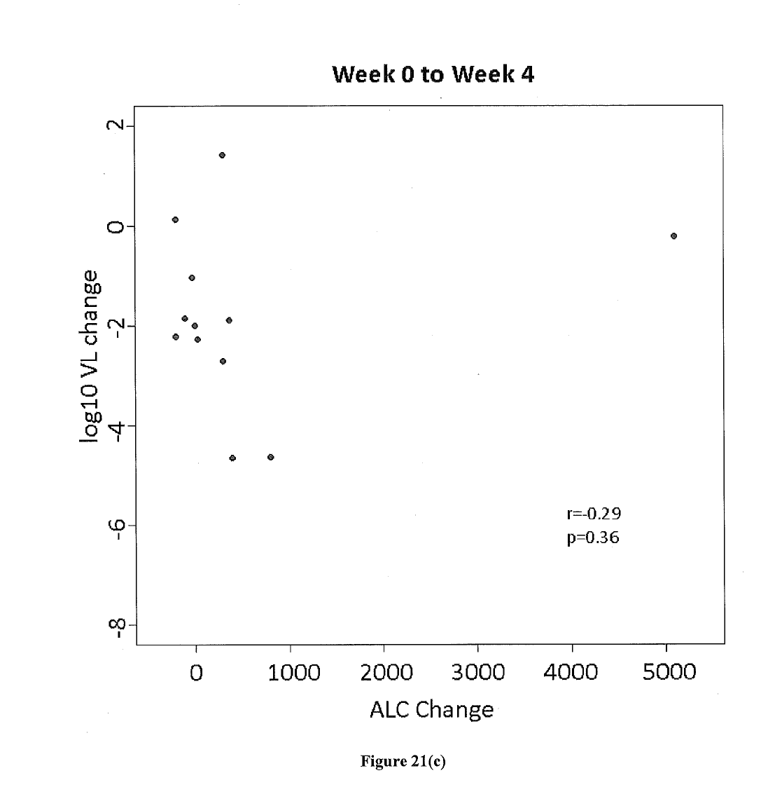

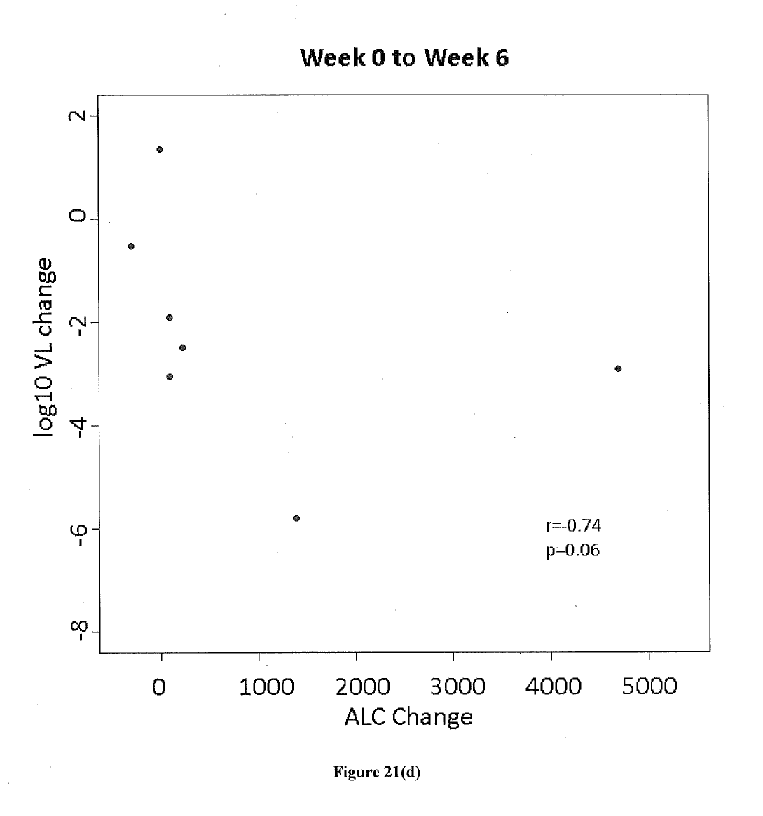

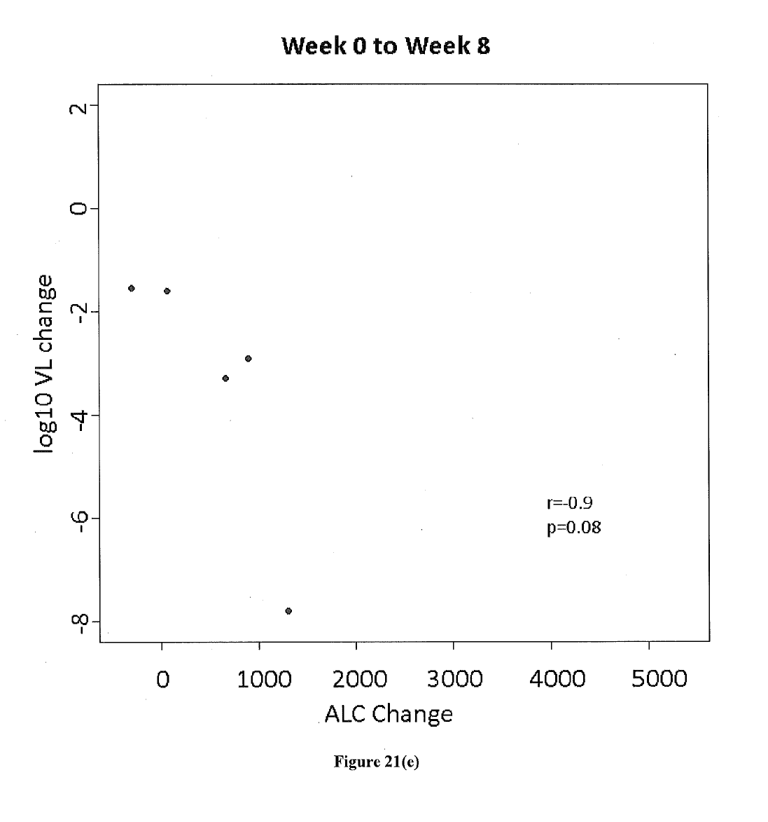

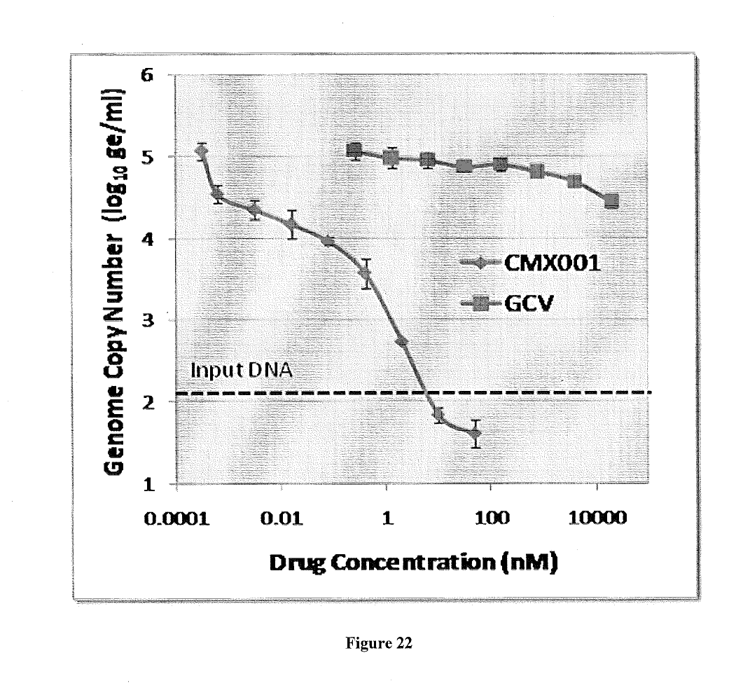

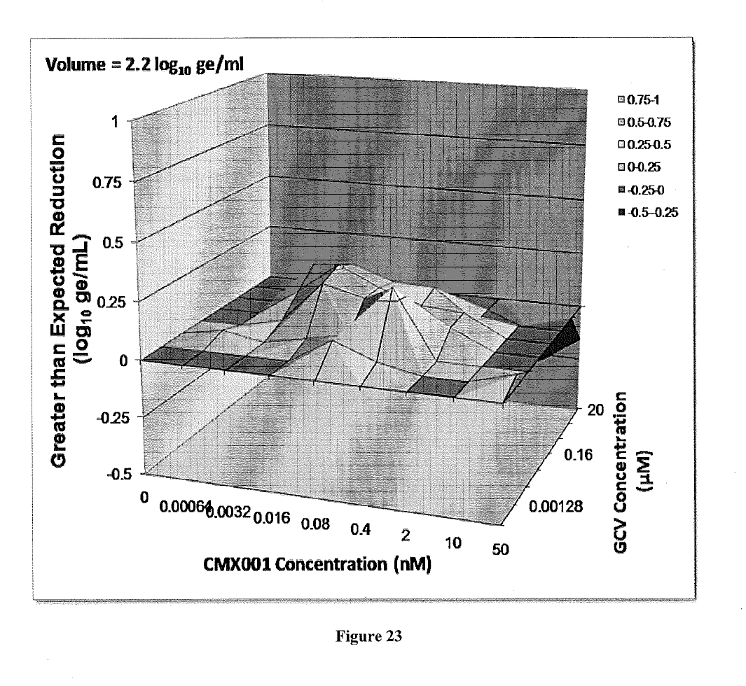

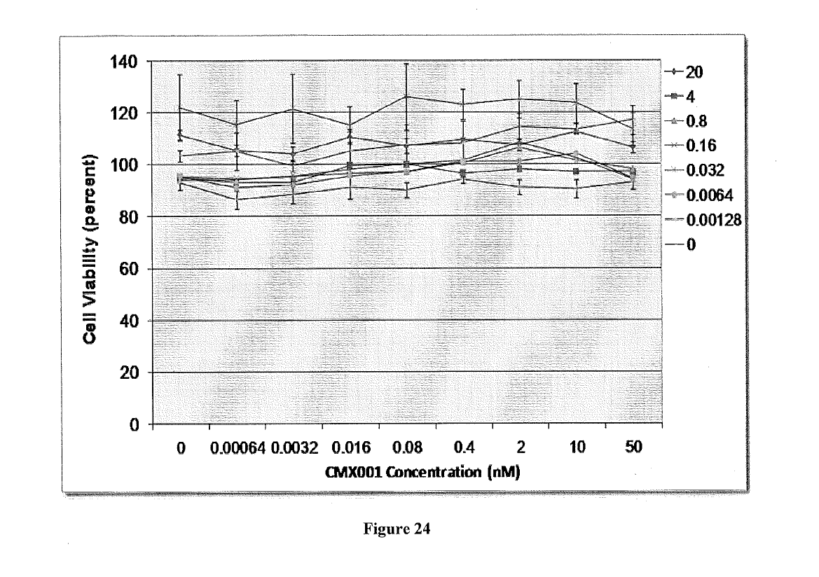

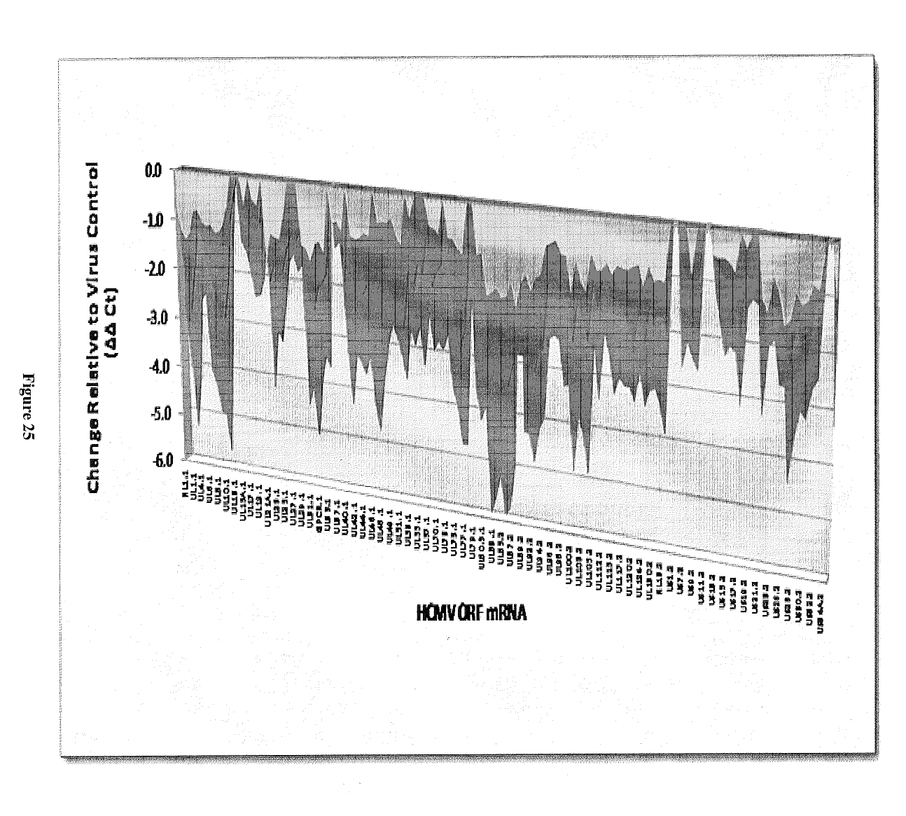

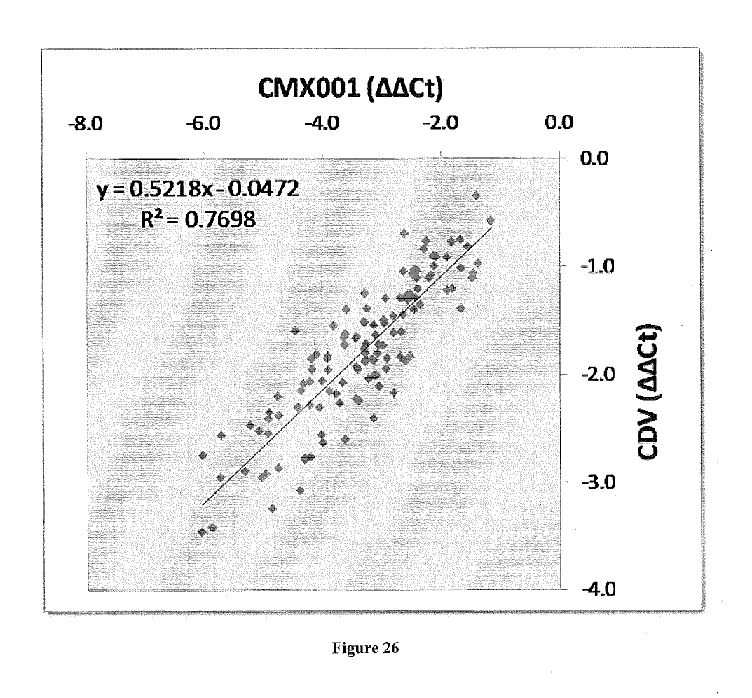

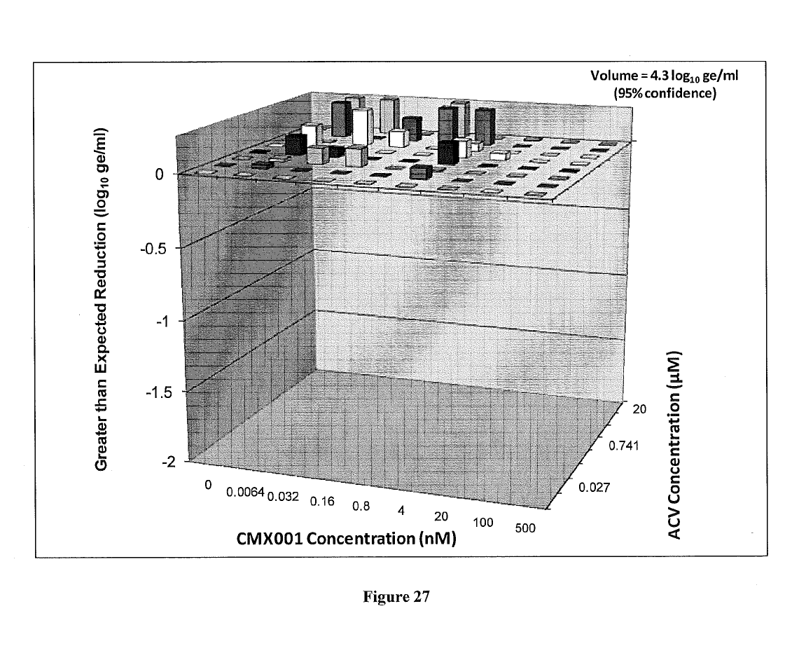

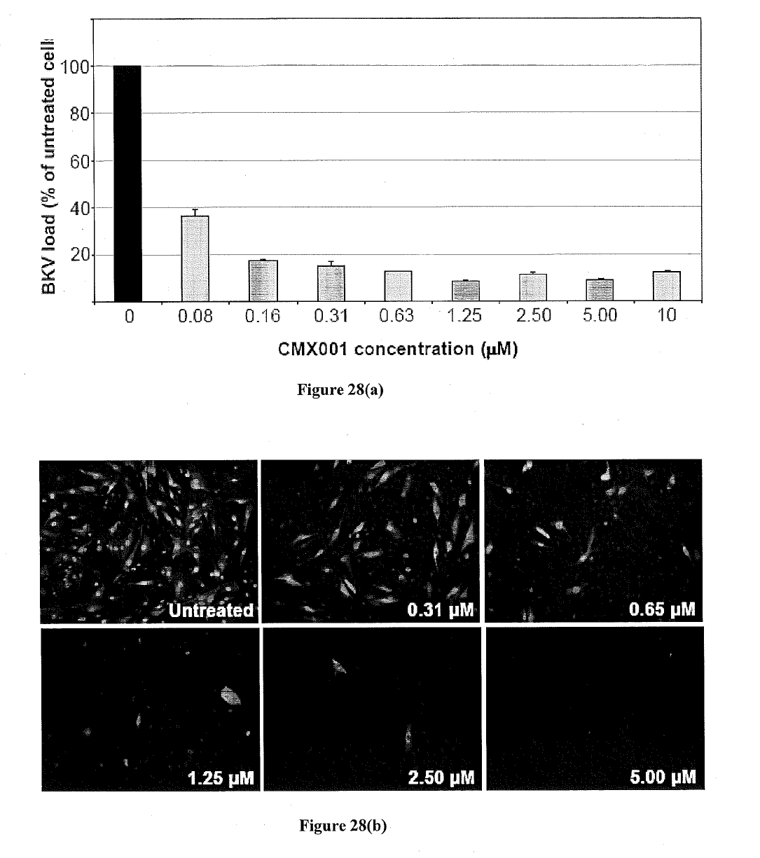

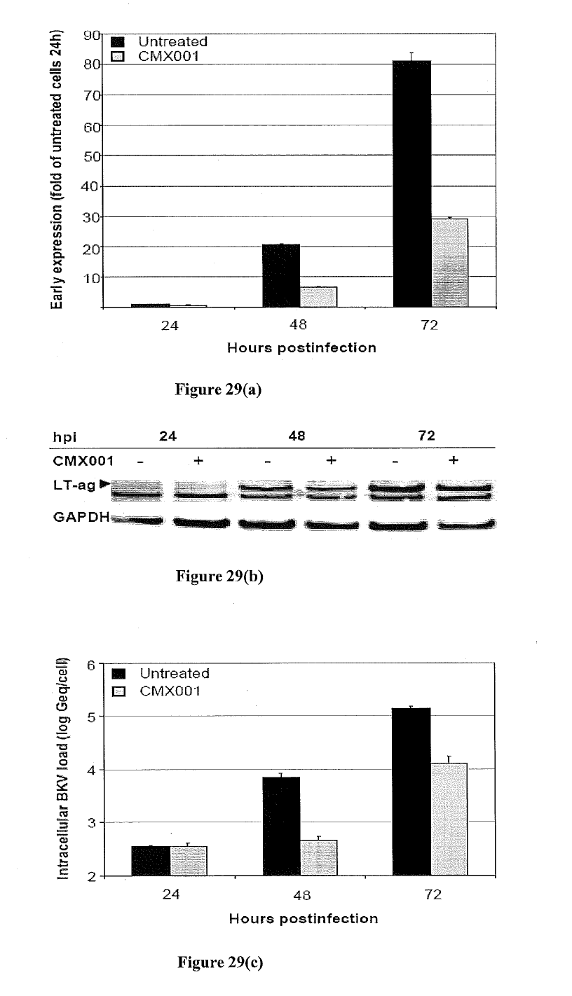

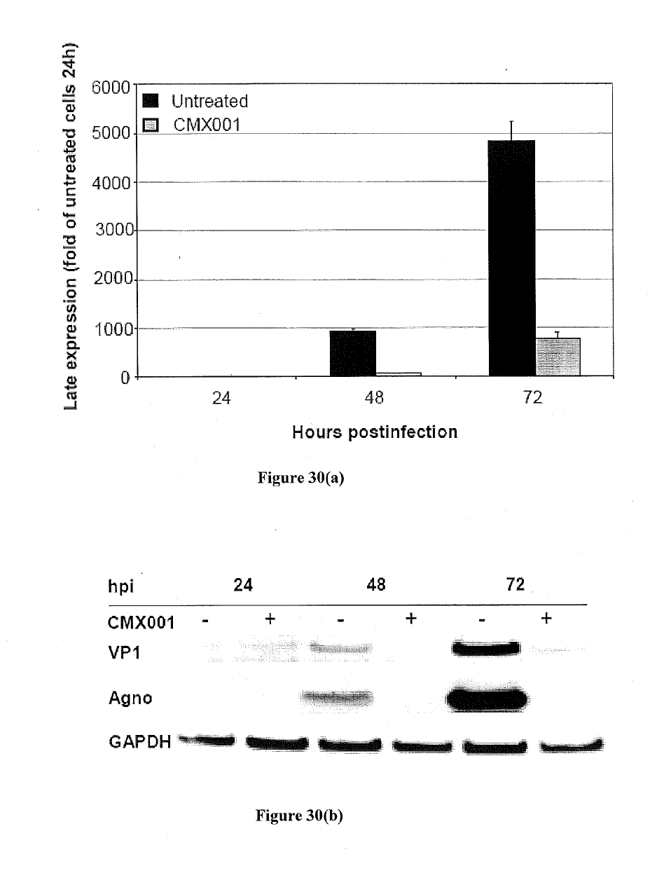

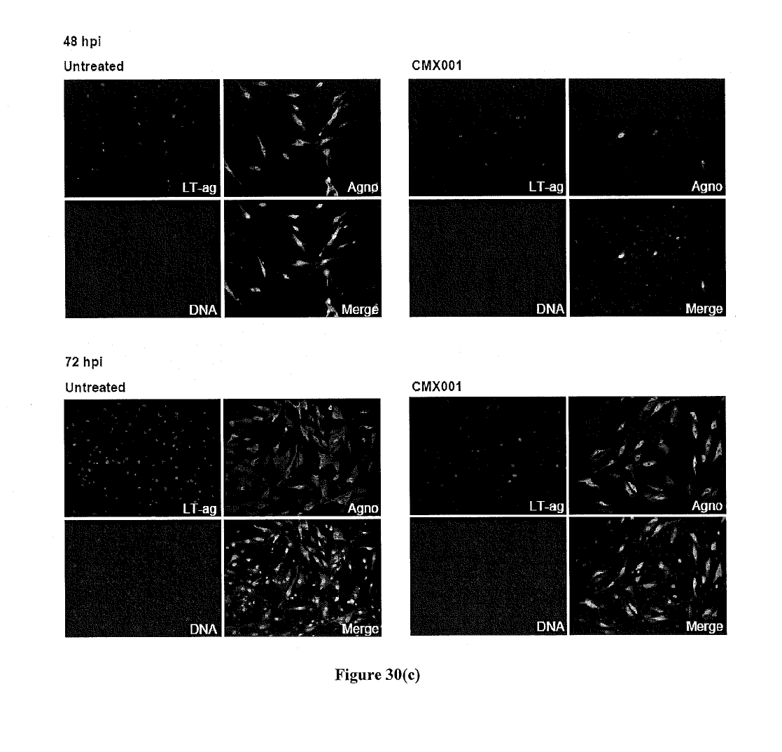

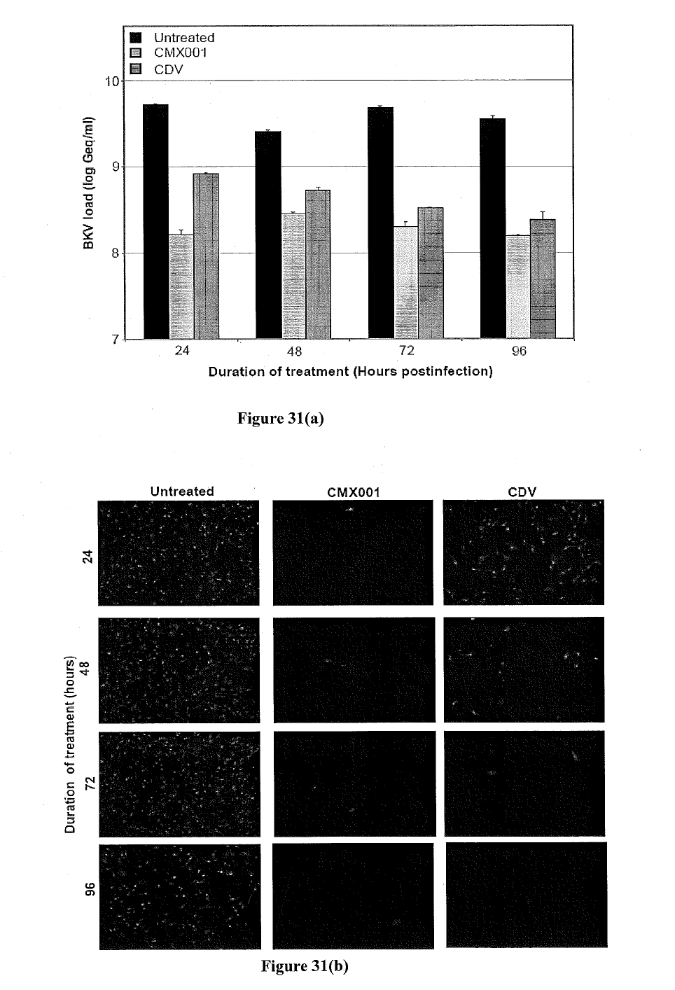

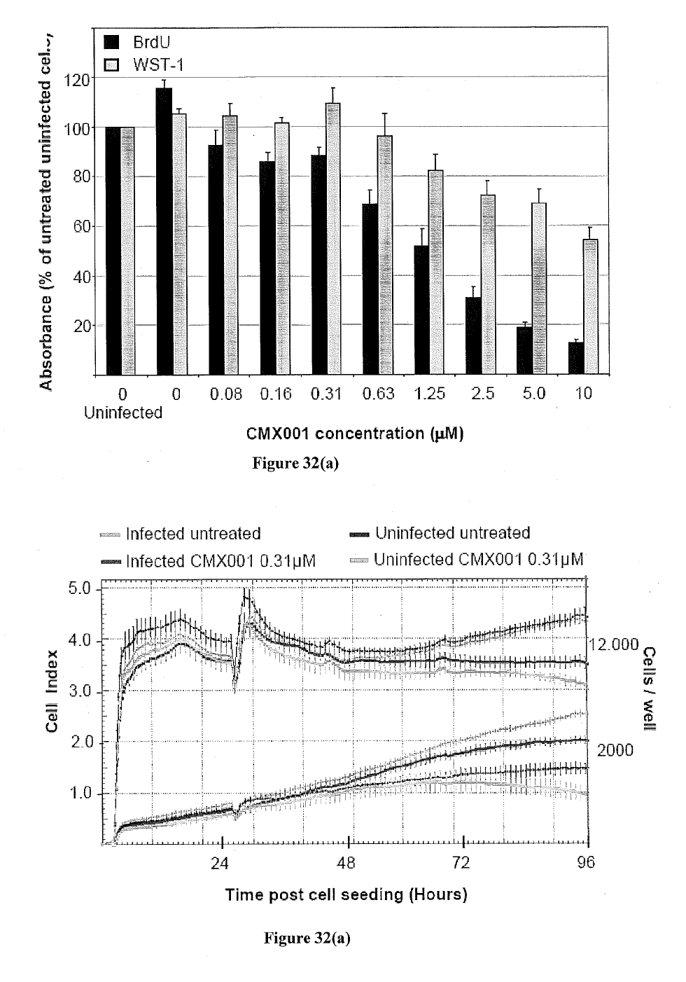

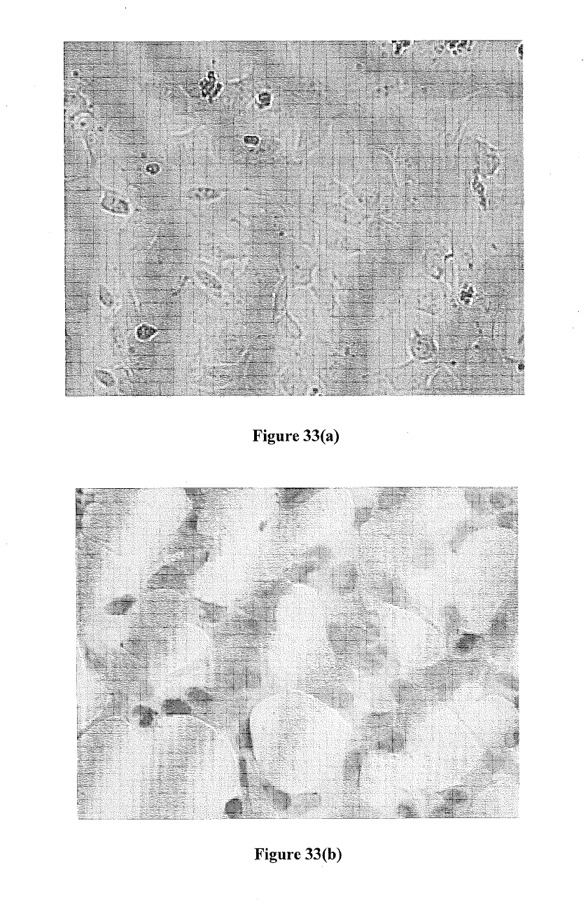

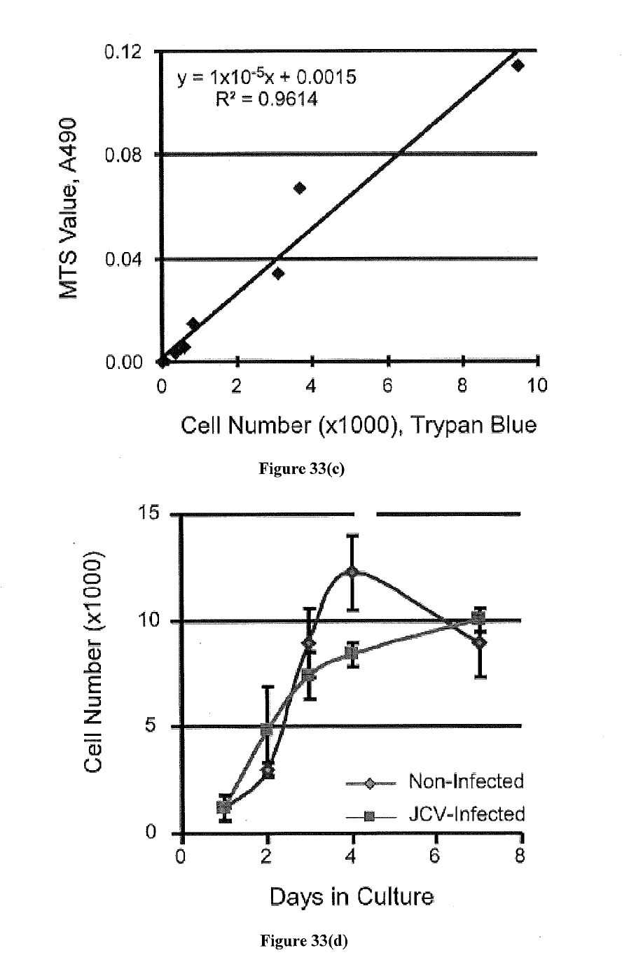

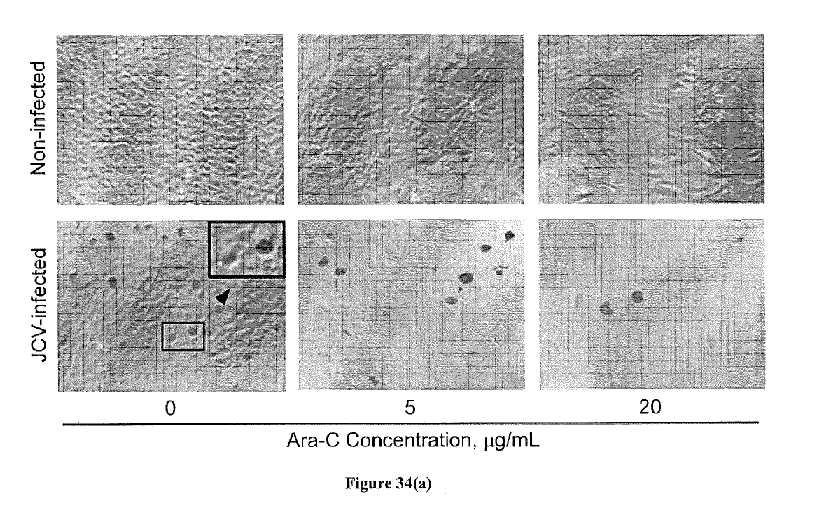

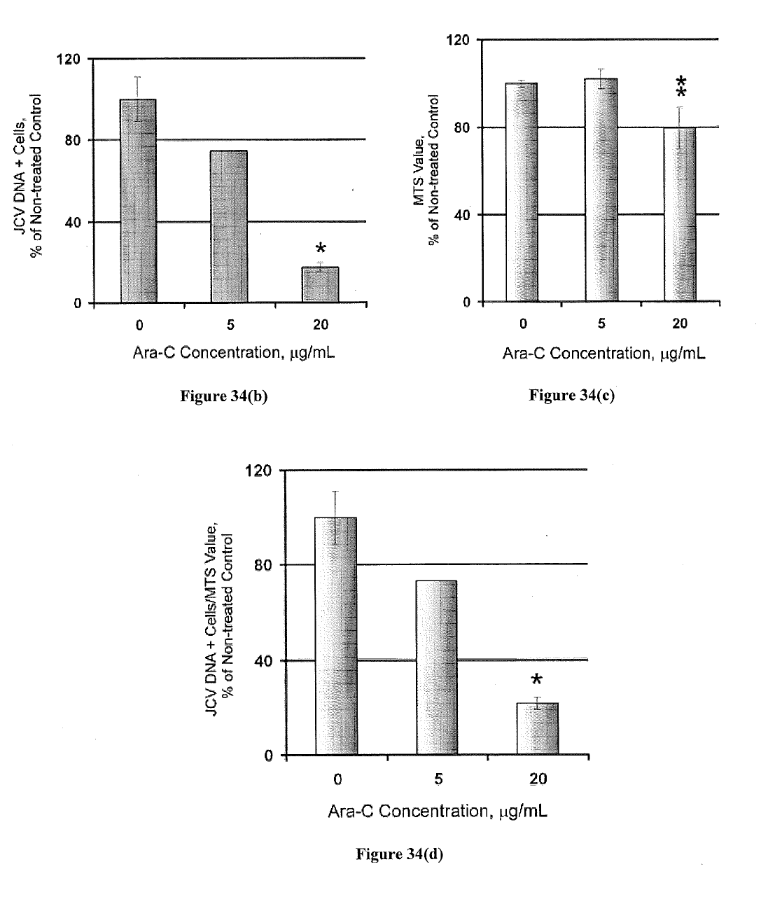

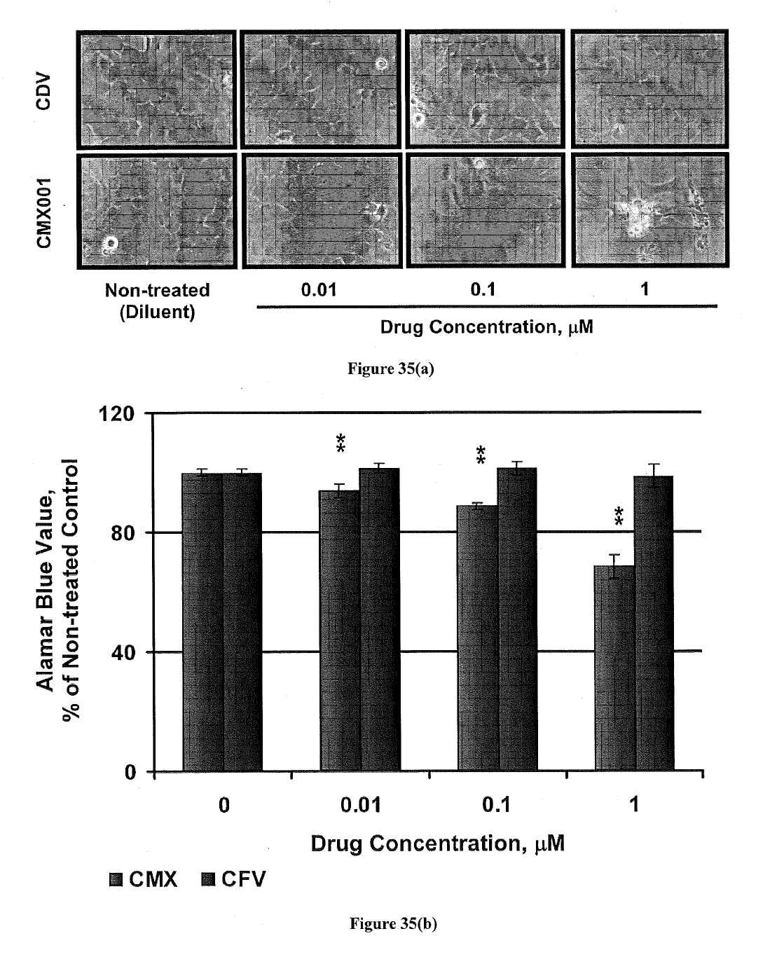

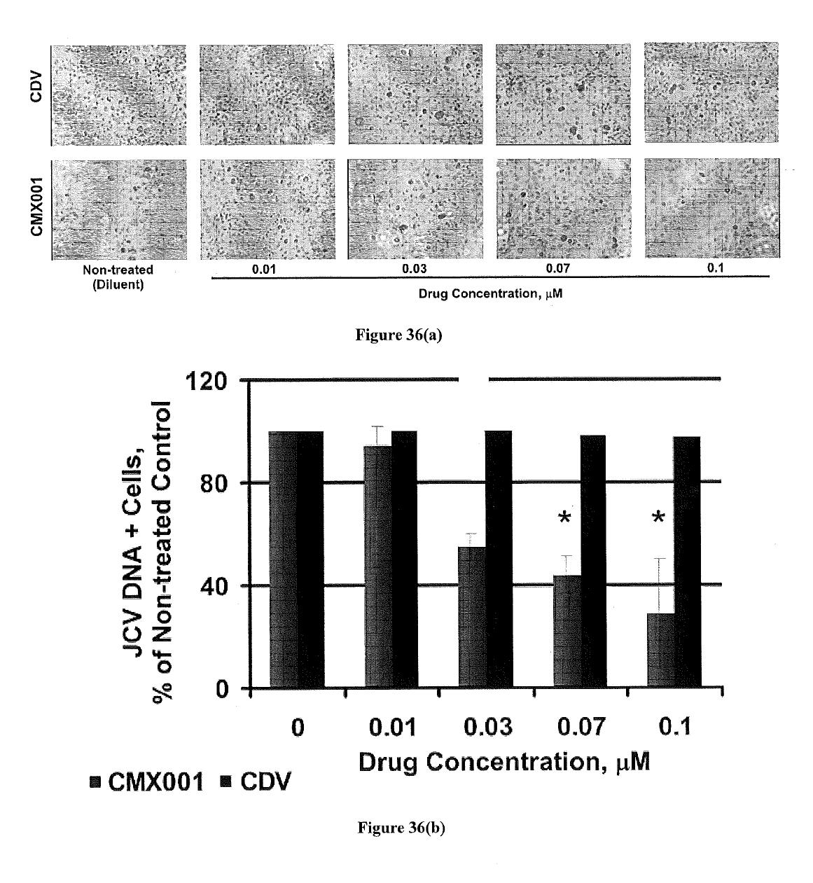

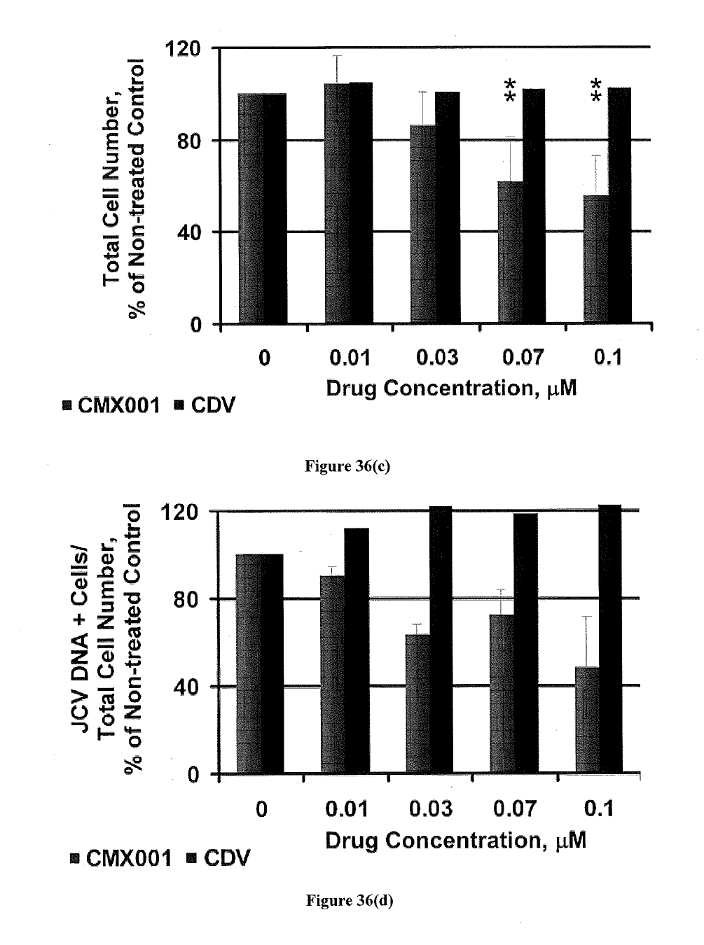

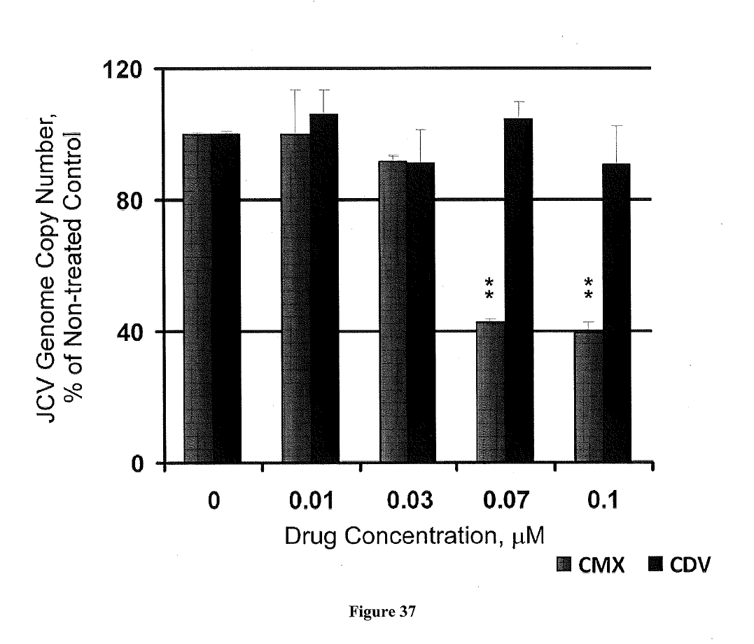

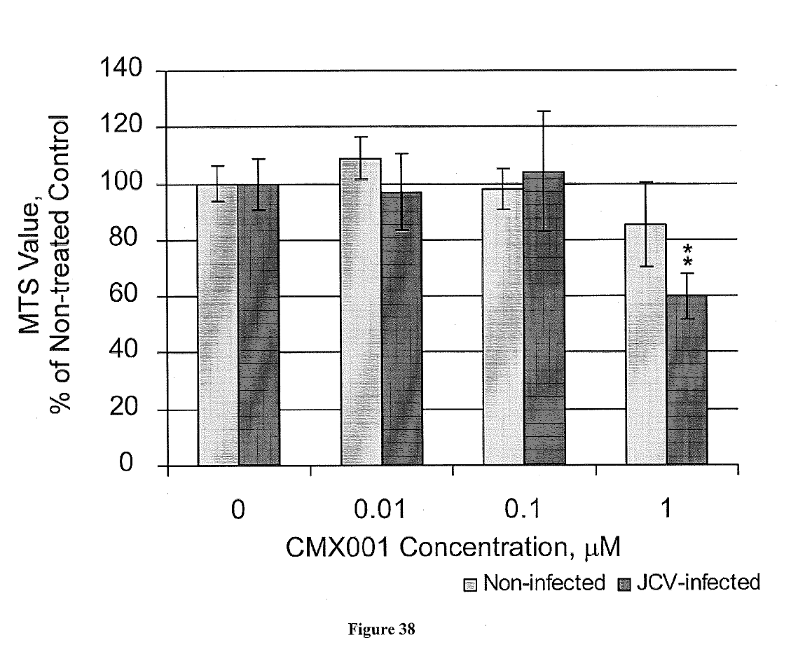

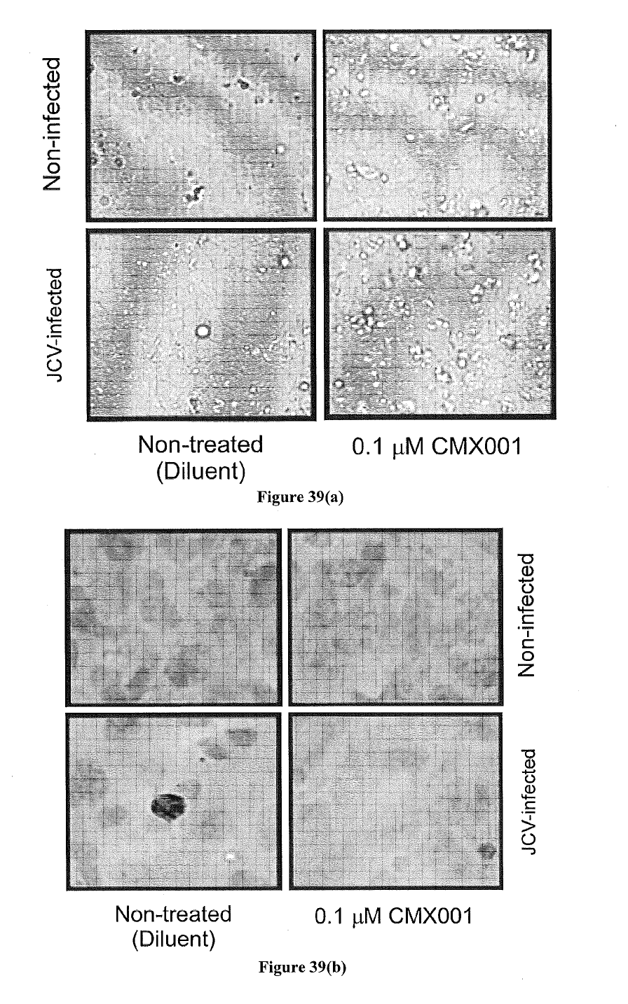

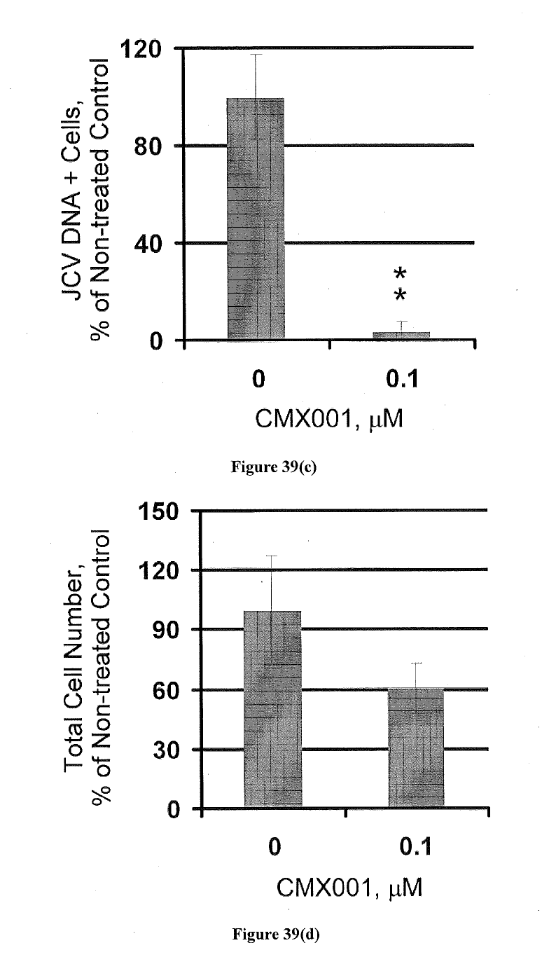

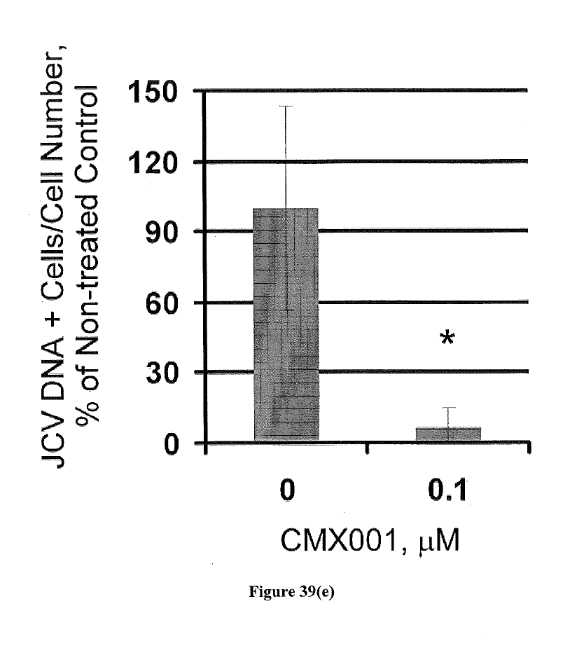

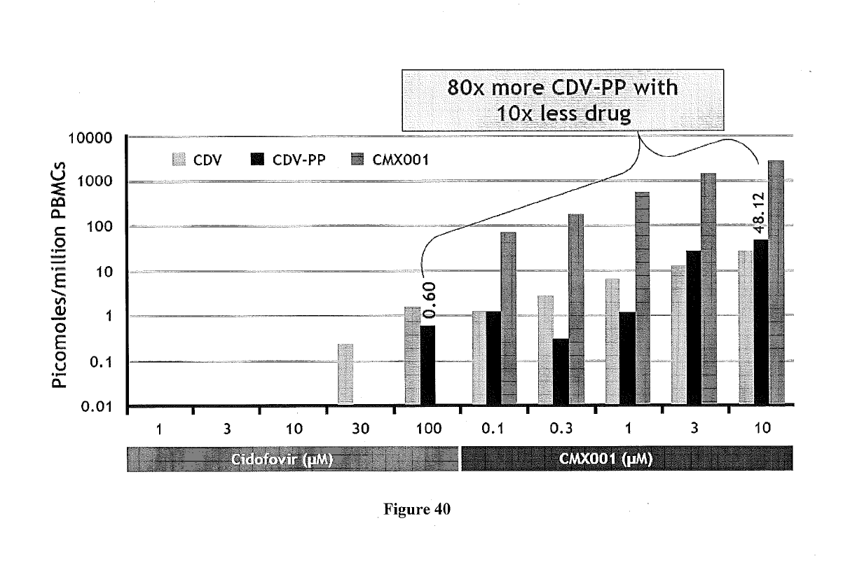

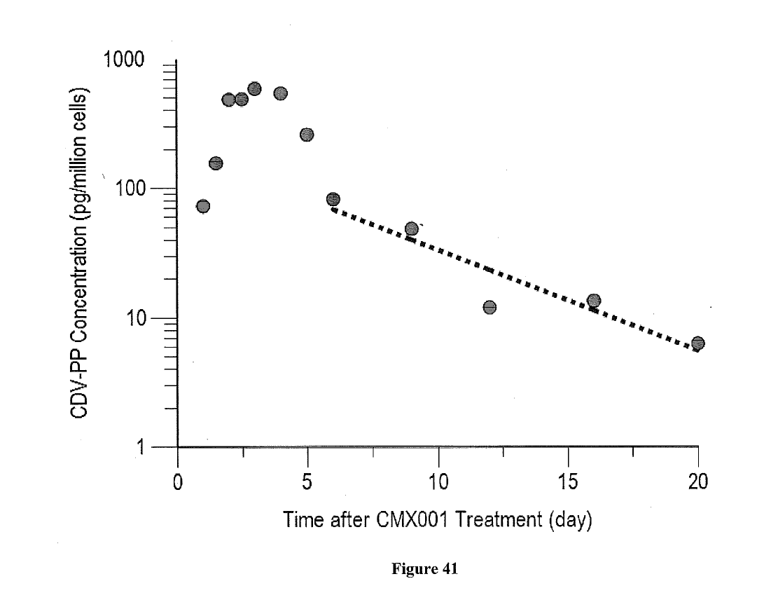

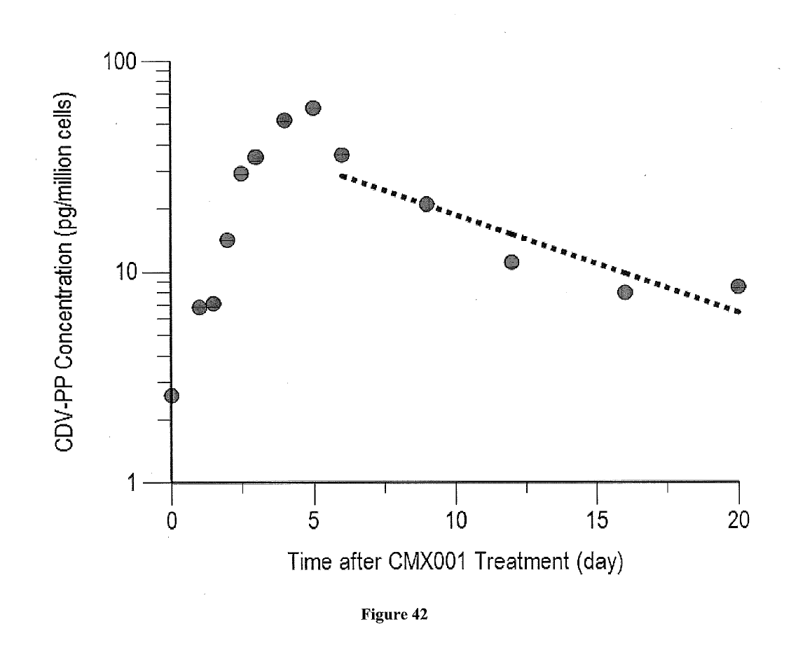

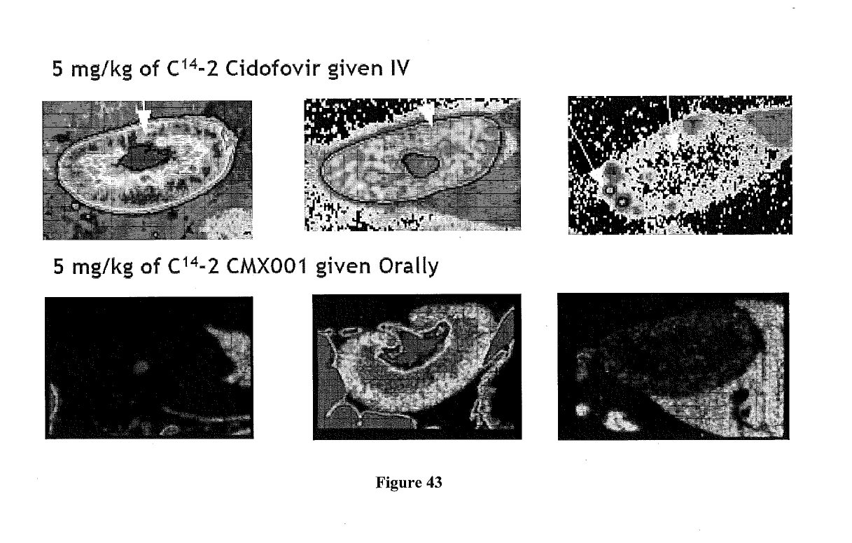

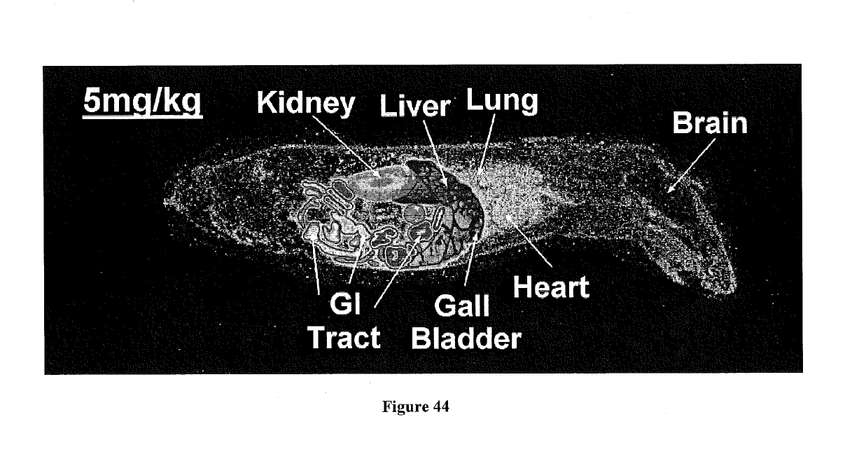

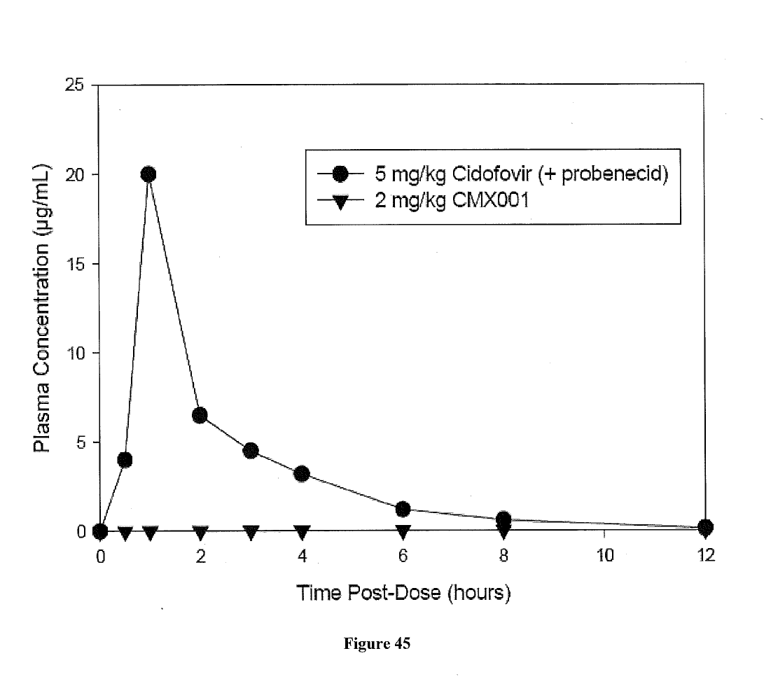

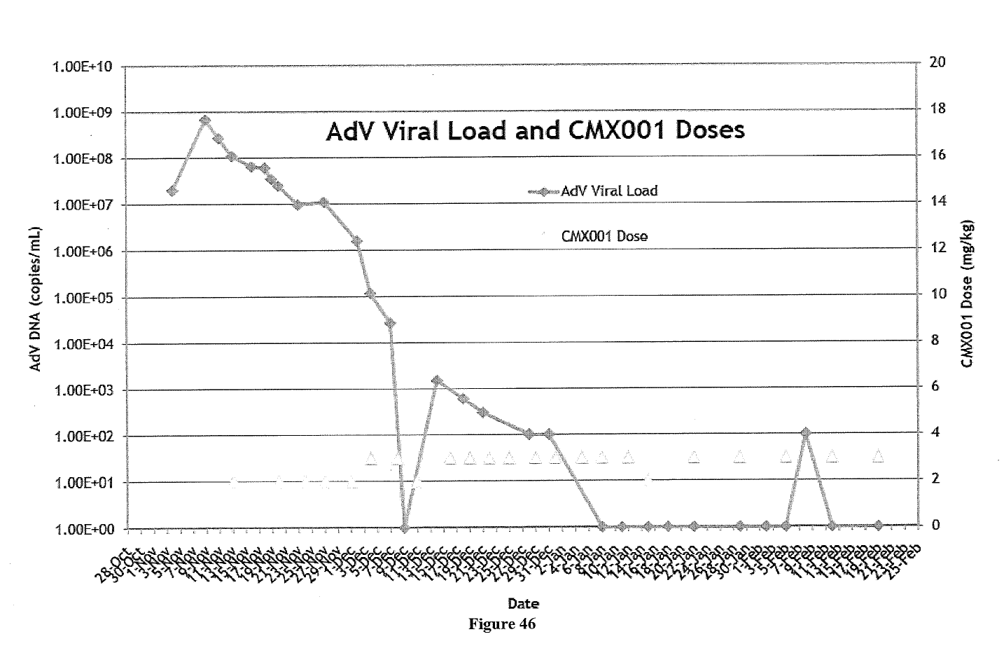

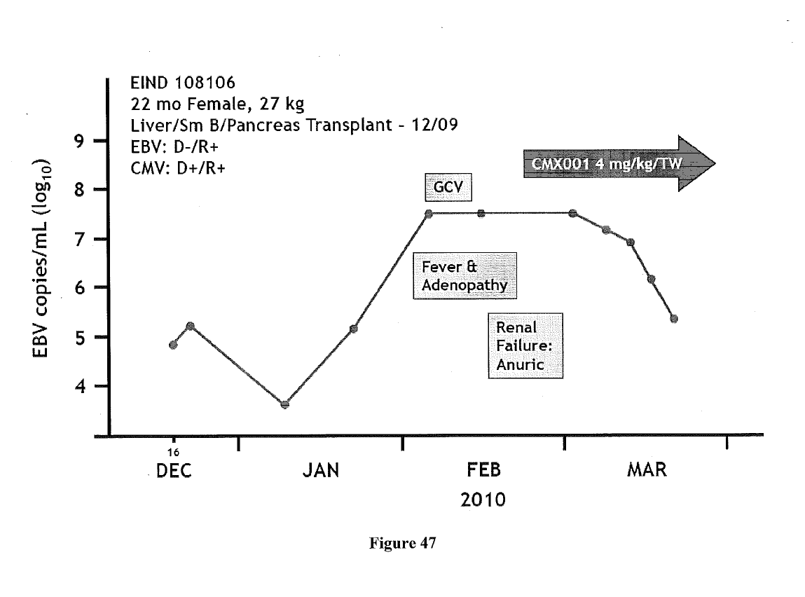

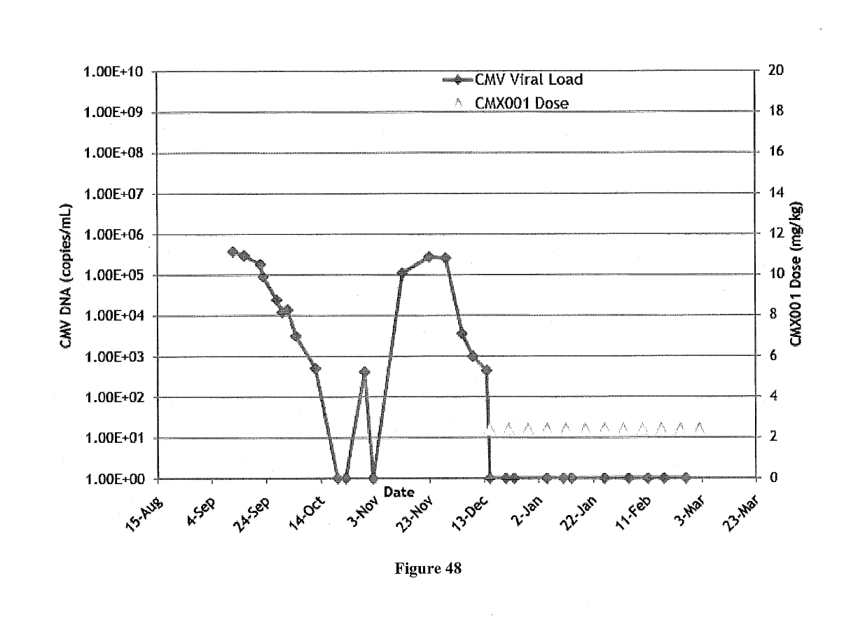

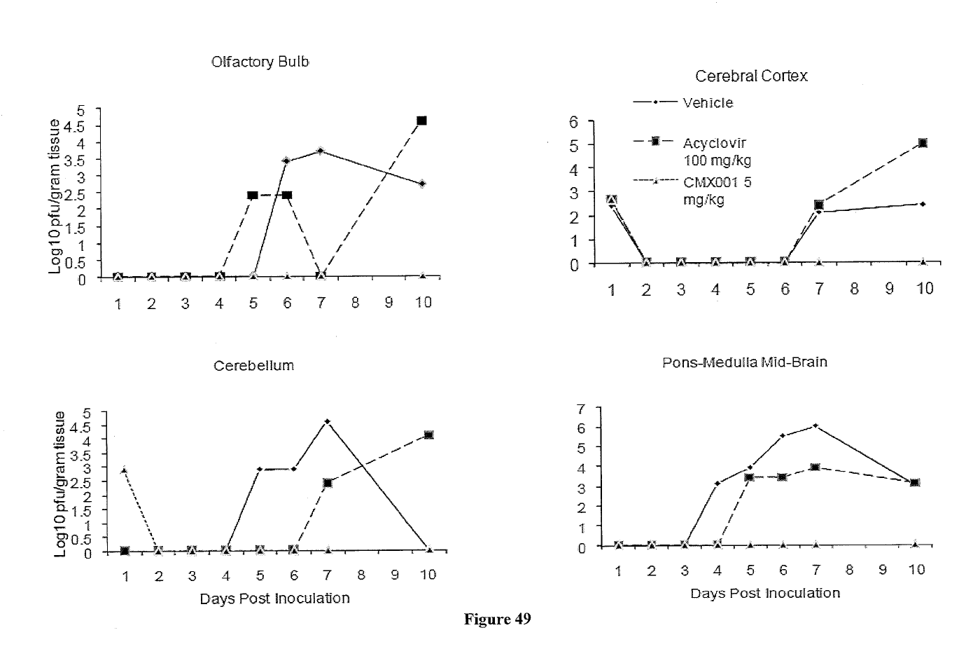

For FIG. 1(a), RPTEC supernatants were harvested 72 h p.i. i.e. 70 h post start of treatment with indicated CMX001 concentrations. BKV load was measured by qPCR and input virus subtracted. DNA load in untreated cells (1.05E+09 Geq/ml) was set as 100%.

For FIG. 1(b), indirect immunofluorescence of untreated and CMX001 treated BKV-infected RPTECs, methanol fixed 72 h p.i. and stained with rabbit anti-agnoprotein serum (green) for visualization of the late agnoprotein and with the SV40 LTag monoclonal Pab416 for visualization of BKV LTag (red). Cell density is shown by Drac 5 staining in blue.

FIG. 2(a) illustrates the relationship between the concentration of CMX001 and DNA replication of uninfected RPTEC. FIG. 2(b) illustrates the relationship between the concentration of CMX001 and metabolic activity of uninfected RPTEC.

For FIG. 2(a), cellular DNA replication was examined with BrdU incorporation. Medium with indicated CMX001 concentrations was added 2 h p.i. and absorbance measured 72 h p.i. Absorbance for untreated cells were set as 100%.

For FIG. 2(b) metabolic activity was examined as WST-1 cleavage. Medium with indicated CMX001 concentrations was added 2 h p.i. and absorbance measured 72 h p.i. Absorbance for untreated cells was set as 100%.

FIG. 3 illustrates the influence of CMX001 0.31 μM on BKV genome replication. For FIG. 3, CMX001-treated and untreated BKV-infected RPTECs were harvested at indicated timepoints and intracellular BKV DNA load per cell was measured by qPCR.

FIG. 4 illustrates the influence of CMX001 0.31 μM on BKV early and late expression. FIG. 4(a) shows the image of indirect immunofluorescence of untreated and CMX001 treated BKV-infected RPTECs. FIG. 4(b) shows the cell extracts from CMX001-treated and untreated BKV-infected RPTECs harvested 48 and 72 h p.i. and western blot performed with rabbit anti-BKV VP1, anti-agnoprotein serum and a monoclonal antibody directed against the housekeeping protein GAPDH.

For FIG. 4(a), indirect immunofluorescence of untreated and CMX001 treated BKV-infected RPTECs, methanol fixed 48 and 72 h p.i. and stained with rabbit anti-agnoprotein serum (green) for visualization of the late agnoprotein and with the SV40 LTag monoclonal Pab416 for visualization of BKV LTag (red). For FIG. 4(b), cell extracts from CMX001-treated and untreated BKV-infected RPTECs were harvested 48 and 72 h p.i. and western blot performed with rabbit anti-BKV VP1, anti-agnoprotein serum and a monoclonal antibody directed against the housekeeping protein GAPDH.

FIG. 5 illustrates the influence of CMX001 0.31 μM on BKV extracellular BKV load. FIG. 5 demonstrates influence of CMX001 0.31 μM on BKV extracellular BKV load, Supernatants from CMX001-treated and untreated BKV-infected RPTECs were harvested at indicated timepoints after infection and BKV load measured by qPCR. Data are presented as BKV load in Geq/ml.

FIG. 6 demonstrates the impact of CMX001 for pre-treatment of RPTECs before infection. For FIG. 6, RPTECs were either treated for 4 hours until 20 h pre-infection when new complete growth medium was added, or they were treated for 24 hours until one hour before infection when they were washed for one hour in complete growth medium before infection. Supernatants were harvested 72 h p.i. and extracellular BKV load measured by qPCR. Data are presented in percent of untreated cells set at 100%.

FIG. 7 illustrates the stability of CMX001. For FIG. 7, BKV-infected RPTECs were treated with freshly made CMX001 or CMX001 from a stock solution at 1 mg/ml stored for one week at 4° C. or −20° C. Supernatants were harvested 72 h p.i. and extracellular BKV load measured by qPCR. Data are presented as BKV load in percent of untreated cells set at 100%.

FIG. 8 demonstrates the replication of JCV Mad-4 in COS-7 cells. For FIG. 8, Indirect immunofluorescence of JCV infected COS-7 cells, fixed 7 d.p.i. and stained with rabbit anti-VP1 serum (red) for visualization of the late capsid protein VP1 and with Höchst 33342 dye to show DNA (blue). The merged pictures are shown in the right panel.

FIG. 9 shows replication of JCV Mad-4 in astrocyte cells and the image of indirect immunofluorescence of JCV-infected astrocyte cells. Fixation and staining are the same as in FIG. 8.

FIG. 10 demonstrates the course of JCV replication in astrocyte cells. FIG. 10(a) shows the indirect immunofluorescence of JCV-infected astrocyte cells, fixed 7 d.p.i. and stained with rabbit anti-VP1 serum (red) for visualization of the late capsid protein VP1 and with Höchst 33342 dye to show DNA (blue). The merged pictures are shown in the right panel. FIG. 10(b) shows the image of indirect immunofluorescence of JCV infected astrocyte cells, fixed at 14 d.p.i. Fixation and staining as in a). FIG. 10(c) shows the image of indirect immunofluorescence of mock-infected astrocyte cells, fixed at 14 d.p.i. Fixation and staining as in 10(a).

FIG. 11 demonstrates the replication of religated JCV Mad-4 DNA in COS-7 cells and the image of indirect immunofluorescence of JCV DNA transfected COS-7 cells, fixed 7 d.p.i. and stained with rabbit anti-VP1 serum (red) for visualization of the late capsid protein VP1 and with Höchst 33342 dye to show DNA (blue). The merged pictures are shown in the right panel.

FIG. 12 demonstrates that the determination of CMX001 IC-50 and IC-90. COS-7 supernatants were harvested 5 d.p.i., i.e. 118 h post start of treatment with indicated CMX001 concentrations. JCV load was measured by qPCR and input virus subtracted. DNA load in untreated cells (5.04×10 E+9 geq/ml) was set as 100%. Replication of JCV is shown as percentage of untreated cells to determine the IC-50 and IC-90.

FIG. 13 demonstrates the effect of increasing concentrations of CMX001 on metabolic activity of COS-7 cells. Metabolic activity was examined as WST-1 cleavage. Medium with indicated CMX001 concentrations was added to COS-7 cells and absorbance measured 72 h post seeding. Absorbance for untreated cells was set as 100%.

FIG. 14 illustrates the effect of increasing concentrations of CMX001 on replication of COS-7 cells. DNA replication was determined by BrdU incorporation. Medium with indicated CMX001 concentrations was added to COS-7 cells and absorbance measured 72 h post seeding. Absorbance for untreated cells was set as 100%.

FIG. 15 shows the effect of increasing concentrations of CMX001 on extracellular viral load. Supernatants from CMX001-treated and untreated JCV-infected COS-7 cells were harvested at indicated timepoints after infection and JCV load measured by qPCR. Data are presented as JCV load in log geq/ml.

FIG. 16 illustrates the effect of increasing concentrations of CMX001 on expression of JCV proteins. Indirect immunofluorescence of JCV-infected COS-7 cells treated with indicated concentrations of CMX001, fixed 7 d.p.i. and stained with rabbit anti-VP1 serum (red) for visualization of the late capsid protein VP1 and with Höchst 33342 dye to show DNA (blue). The merged pictures are shown in the right panel.

FIG. 17 illustrates the effect of increasing concentrations of CMX001 on extracellular viral load in astrocytes. Supernatants from JCV-infected PDA cells treated with indicated concentrations of CMX001 were harvested at indicated timepoints after infection and JCV load measured by qPCR. Data are presented as JCV load in log geq/ml.

FIG. 18 illustrates plasma concentration curves of CMX001 following a single dose administration.

FIG. 19 illustrates plasma concentration curves of Cidofovir following a single dose of CMX001.

FIG. 20 illustrates plasma adenovirus immediately prior to and during treatment with CMX001. Treatment initiated at 2 mg/kg administered twice weekly increasing to 3 mg/kg after the 6^{th} dose. After the virus became undetectable (<10²), administration of CMX001 continued at 3 mg/kg but the schedule was reduced to once weekly for maintenance. The inset shows dose normalized maximum plasma concentrations (Cmax) and systemic exposure (AUCO-int) of CMX001 after the 1^{st}, 10^{th} and 20^{th} doses in comparison to healthy volunteers (HVT) administered a single dose.

FIGS. 21a-21e illustrate scatterplots of change from baseline in log 10 viral load (y-axis) vs. ALC (x-axis). Plots show the difference from week 0 to weeks 1, 2, 4, 6, and 8, respectively. Spearman correlation coefficients and p-values are included.

FIG. 22 illustrates that CMX001 and GCV inhibit the accumulation of viral DNA. Monolayers of HFF cells in 96-well plates were infected with HCMV at and MOI of 0.001 PFU/cell. Compound dilutions were added and infected cells were incubated for 7 days. Total DNA was purified and quantified by real time PCR and is given as log₁₀ genome equivalents/ml of culture (log₁₀ ge/ml). Values represent the average of 4 wells and the bars represent the standard deviation of the data. The dashed line represents the input DNA associated with the inoculum used to initiate the infection.

FIG. 23 illustrates that CMX001 and GCV synergistically inhibit the replication of HCMV. Compounds were added to infected cells at the concentrations shown. Data were derived from the genome copy number determined from 4 replicate samples. The synergy plot represents greater than expected inhibition viral replication at each combination of concentrations at the 95% confidence level. The volume of synergy was relatively low (2,2 log₁₀ genome equivalents/ml (log₁₀ ge/ml), but occurred at a broad range of concentrations.

FIG. 24 illustrates the combined cytotoxicity of CMX001 and GCV in HFF cells. Cell viability was determined at 7 days following the addition of drug combinations. Data shown is the viability at each concentration of CMX001 (nM), with the addition of GCV at the concentration shown in the figure legend (μM). Error bars represent the standard deviation of two replicate determinations.

FIG. 25 illustrates that GCV, CDV, and CMX001 reduce quantities of HCMV transcripts.

FIG. 26 illustrates that transcriptional responses to CDV and CMX001 are similar.

FIG. 27 illustrates a synergy plot of CMX001 and ACV combinations in vitro.

FIG. 28 illustrates the effect of increasing concentrations of CMX001 on BKV load and expression of BKV proteins. FIG. 28(a) RPTEC supernatants were harvested 72 hpi i.e. 70 h post start of treatment with indicated CMX001 concentrations and BKV load was measured by qPCR. DNA load in untreated cells (1.19E+09 Geq/ml) was set as 100%. FIG. 28(b) indirect immunofluorescence of BKV-infected RPTECs either untreated or treated with indicated CMX001 concentrations. The cells were methanol fixed 72 hpi and stained using as primary antibodies polyclonal rabbit anti-agno serum (green) for visualization of the late agno and the SV40 LT-ag monoclonal Pab416 for visualization of BKV LT-ag (red). Cell nuclei (blue) were stained with Drac 5.

FIG. 29 illustrates the influence of CMX001 at 0.31 μM on the BKV-Dunlop early expression and DNA replication in RPTECs. FIG. 29(a) Early mRNA expression. RNA was extracted from CMX001-treated and untreated BKV-infected RPTECs at indicated timepoints. LT-ag mRNA expression was measured by RT-qPCR and normalized to huHPRT transcripts. Results are presented as changes in the LT-ag mRNA level, with the level in the untreated sample at 24 h p.i arbitrarily set to 1. FIG. 29(b) Early protein expression. Cell extracts from CMX001-treated and untreated BKV-infected RPTECs were harvested 24, 48 and 72 hpi and western blot performed with polyclonal rabbit anti-LT-ag serum and with a monoclonal antibody directed against the housekeeping protein glyceraldehydes-3-phosphate dehydrogenase (GAPDH). The anti-LT-ag serum also recognize a cellular protein of unknown origin. FIG. 29(c) BKV DNA replication. CMX001-treated and untreated BKV-infected RPTECs were harvested at indicated timepoints and DNA extracted. Intracellular BKV DNA load was measured by qPCR and normalized for cellular DNA using the aspartoacyclase (ACY) qPCR. Data are presented as Geq/cell.

FIG. 30 illustrates the influence of CMX001 at 0.31 μM on the BKV-Dunlop late expression in RPTECs late mRNA expression. FIG. 30(a) Late mRNA expression. RNA was extracted from CMX001-treated and untreated BKV-infected RPTECS at indicated timepoints. VP1 mRNA expression was measured by RT-OCR and normalized to huHPRT transcripts. Results are presented as changes in the VP1 mRNA level, with the level in the untreated sample at 24 hpi arbitrarily set to 1. FIG. 30(b) Late protein expression. Cell extracts from CMX001-treated and untreated BKV-infected RPTECs were harvested 24, 48 and 72 hpi and western blot performed with polyclonal rabbit anti-agno and anti-VP1 serum and with the monoclonal antibody anti-GAPDH. FIG. 30(c) Early and late protein expression. Indirect immunofluorescence of BKV-infected RPTECs either untreated or treated with CMX001. The cells were methanol fixed 48 and 72 hpi and stained using as primary antibodies polyclonal rabbit anti-agno serum (green) for visualization of the late agno and the SV40 LT-ag monoclonal Pab416 for visualization of BKV LT-ag (red). Cell nuclei (blue) were stained with Drac 5.

FIG. 31 illustrates the kinetics of CMX001 at 0.31 μM treatment of BKV-infected RPTECs. FIG. 31(a) Extracellular BKV load. Two-hours after infection, CMX001 was added and treatment continued for 24, 48, 72 or 96 h, respectively. At the indicated time supernatant was removed, cells were washed and new medium added. At 96 hpi all supernatants were harvested and OCR was performed. Data are presented as BKV load in Geq/ml. FIG. 31(b) The supernatant collected 96 hpi from the cells described above, where diluted 1:10 and seeded on new RPTEC cells. 72 hpi cells were methanol fixed and immunofluoresence staining with polyclonal rabbit anti-agno serum (green) and the SV40 LT-ag monoclonal Pab416 was performed (red). Cell nuclei (blue) were stained with Drac 5.

FIG. 32 illustrates the influence of CMX001 on DNA replication, metabolic activity, cell adhesion and proliferation of uninfected and BKV-infected RPTECs. FIG. 32(a) Cellular DNA replication was examined with a cell proliferation enzyme-linked immunosorbent assay (ELISA) monitoring BrdU incorporation and metabolic activity was examined with cell proliferation reagent WST-1 measuring WST-1 cleavage. Medium with indicated CMX001 concentrations was added 2 hpi and absorbance measured 72 hpi Absorbance for untreated uninfected cells was set as 100%. FIG. 32(b) For a dynamic monitoring of cell adhesion and proliferation of RPTECs the XCELLigence system was used. RPTECs at a density of 2000 cells/well and 12,000 cells/well were seeded on E-plates. Twenty-seven hours post seeding, 150 μl of the media in each well (totally 200 μl) was replaced with fresh media with or without purified BKV-Dunlop(MOI 5) and with or without CMX001 (total concentration of 0.31 μM) and the cells were left until 96 h post cell seeding.

FIG. 33 illustrates the SVG cell growth kinetics. SVG cells growing in culture were analyzed by differential interference contrast microscopy (FIG. 33(a)) and phase contrast microscopy following hematoxylin staining (FIG. 33(b)) at 100× magnification. MTS analysis and trypan blue staining were used to generate a standard curve to correlate cell viability by MTS assay into total cell number (FIG. 33(c)). SVG growth kinetics were measured over 7 days in culture by MTS assay and converted into total cell numbers using the standard curve (FIG. 33(d)).

FIG. 34 illustrates that Ara-C treatment suppresses JCV infection in SVG cells. SVG cells were exposed to 10 HAU of Mad-4 JCV per 5×10⁴ cells overnight. Cells were then treated with 0, 5, or 20 μg per mL of Ara-C. JCV DNA in SVG cells treated with Ara-C was detected by in situ DNA hybridization (FIG. 34(a)). The total number of JCV DNA containing cells was quantified for each concentration of Ara-C tested and is expressed as a percentage of the non-treated control (FIG. 34(b)). Cell density for the SVG cells processed for in situ DNA hybridization was determined by semi-quantification of hematoxylin intensity and is expressed as a percentage of the non-treated control (FIG. 34(c)). The total number of JCV DNA containing cells was normalized for cell density and is expressed as a percentage of the non-treated control (FIG. 34(d)). Error bars represent standard deviation. A single asterisk represents a p<0.05 and two asterisks represent a p<0.01.

FIG. 35 illustrates the limited cytotoxicity of CMX001 to SVG cells. SVG cells growing in culture were treated with drug diluent or 0.01, 0.1 and 1 μM CMX001 or CDV. CMX001 or CDV treated cells were analyzed by phase contrast microscopy at 100× magnification (FIG. 35(a)). Cell viabilities of CMX001 or CDV treated cells were determined by alamar blue staining and are expressed as a percentage of the non-treated control (FIG. 35(b)). Error bars represent standard deviation. Two asterisks represent a p<0.01.

FIG. 36 illustrates that CMX001 suppresses JCV replication in SVG cells. SVG cells were exposed to 10 HAU of Mad-4 JCV per 5×10⁴ cells overnight. Cells were then treated with drug diluent or 0.01, 0.03, 0.07, or 0.1 μM CMX001 or CDV. JCV DNA in infected SVG cells was detected by in situ DNA hybridization (FIG. 36(a)). The total number of JCV DNA containing cells was quantified for each concentration of drug tested and is expressed as a percentage of the non-treated control (FIG. 36(b)). Total cell number for the SVG cells processed for in situ DNA hybridization was determined by semi-quantification of hematoxylin intensity and is expressed as a percentage of the non-treated control (FIG. 36(c)). The number of JCV DNA containing cells was normalized for total cell number and is expressed as a percentage of the non-treated control (FIG. 36(d)). Error bars represent standard deviation. A single asterisk represents a p<0.05, and two asterisks represent a p<0.01.

FIG. 37 illustrates that CMX001 reduces JCV DNA replication in SVG cells. SVG cells were exposed to 10 HAU of Mad-4 JCV per 5×10⁴ cells overnight. Cells were then treated with 0, 0.01, 0.03, 0.07, and 0.1 μM of CMX001 or CDV. Total DNA was isolated 4 days after drug-treatment and JCV DNA was detected by quantitative real-time PCR. JCV genome copy number is expressed as a percentage of the non-treated control. Error bars represent standard deviation. Two asterisks represent a p<0.01.

FIG. 38 illustrates the limited cytotoxicity of CMX001 in an established JCV infection of SVG cells. JCV infection was initiated in SVG cells and maintained over 12 passages in culture. Cells were subsequently treated with 0, 0.01, 0.1, and 1 μM CMX001 for four days in culture. Cell viability was measured by MTS assay. Cell viability is expressed as a percentage of the non-treated control. Error bars represent standard deviation. Two asterisks represent a ρ<0.01.

FIG. 39 illustrates that CMX001 treatment eliminates JCV-infected cells from an established infection. JCV infection was initiated in SVG cells and maintained over 8 passages in culture. Cells were subsequently treated with 0 or 0.1 μM CMX001 for four days in culture. CMX001 treated cells were analyzed by phase contrast microscopy at 100× magnification (FIG. 39(a)). JCV DNA in infected SVG cells treated with CMX001 was detected by in situ DNA hybridization (FIG. 39(b)). The total number of JCV DNA containing cells was quantified and is expressed as a percentage of the non-treated control (FIG. 39(c)). The total number of cells for the SVG cells processed for in situ DNA hybridization was determined by semi-quantification of hematoxylin intensity and is expressed as a percentage of the non-treated control (FIG. 39(d)). The total number of JCV DNA containing cells was normalized for cell density and is expressed as a percentage of the non-treated control (FIG. 39(e)). Error bars represent standard deviation. A single asterisk represents a p<0.05, and two asterisks represent a p<0.01.

FIG. 40 illustrates that CMX001 results in 80 times more CDV-PP with 10 times less drug than cidofovir.

FIG. 41 illustrates the in vitro intracellular levels of CDV-PP in human PBMCs after incubation with CMX001 for 48 hours.

FIG. 42 illustrates the in vitro levels of CDV-PP in human PBMCs after incubation with CMX001 for 1 hour.

FIG. 43 illustrates the clearance of cidofovir or CMX001 from mouse kidney over 4 hours.

FIG. 44 illustrates the organ distribution of CMX001 four hours after an oral dose of 5 mg/kg of [C2-¹⁴C] CMX001.

FIG. 45 illustrates the comparison of plasma cidofovir concentrations following IV cidofovir or oral CMX001.

FIG. 46 illustrates a patient's response of adenovirus viremia to CMX001 treatment.

FIG. 47 illustrates the treatment of Epstein-Barr virus (EBV) viremia in a patient with CMX001.

FIG. 48 illustrates a CMX001 dose and plasma CMV by PCR plot.

FIG. 49 illustrates the effects of CMX001 on Herpes simplex virus-2 (HSV-2) replication in the CNS.

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In case of a conflict in terminology, the present specification is controlling.

### A. DEFINITIONS

As used herein, “alkyl” refers to a straight or branched chain hydrocarbon containing from 1 to 30 carbon atoms. In some embodiments, the alkyl group contains 1 to 24, 2 to 25, 2 to 24, 1 to 10, or 1 to 8 carbon atoms. In one embodiment, the alkyl group contains 15, 16, 17, 18, or 19 to 20 carbon atoms. In some embodiments the alkyl group contains 16 or 17 to 20 carbon atoms. In some embodiments, the alkyl group contains15, 16, 17, 18, 19 or 20 carbon atoms. In still other embodiments, alkyl group contains 1-5 carbon atoms, and in yet other embodiments, alkyl group contain 1-4 or 1-3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. Additional examples or generally applicable substituents are illustrated by the specific compounds described herein.

As used herein, “alkenyl,” refers to a straight or branched chain hydrocarbon containing from 2 to 30 carbons and containing at least one carbon-carbon double bond formed by the removal of two hydrogens. In some embodiments, the alkenyl group contains 2 to 25, 2 to 24, 2 to10, 2 to 8 carbon atoms. In one embodiment, the alkenyl group contains 15, 16, 17, 18, 19 to 20 carbon atoms. In some embodiments, the alkenyl group contains 16 or 17 to 20 carbon atoms. In still other embodiments, alkenyl groups contain 15, 16, 17, 18, 19 or 20 carbon atoms, and in yet other embodiments, alkenyl groups contain 2-5, 2-4 or 2-3 carbon atoms. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. Additional examples or generally applicable substituents are illustrated by the specific compounds described herein.

As used herein, “alkynyl,” refers to a straight or branched chain hydrocarbon group containing from 2 to 30 carbon atoms and containing at least one carbon-carbon triple bond. In some embodiments, the alkynyl group contains 2 to 25, 2 to 24, 2 to 10, or 2 to 8 carbon atoms. In one embodiment, the alkynyl group contains 15, 16, 17, 18 or 19 to 20 carbon atoms. In some embodiments, the alkynyl group contains 16 or 17 to 20 carbon atoms. In still other embodiments, alkynyl groups contain 15, 16, 17, 18, 19 or 20 carbon atoms, and in yet other embodiments, alkynyl groups contain 2-5, 2-4 or 2-3 carbon atoms. Representative examples of alkynyl include, but are not limited, to ethynyl, 1-propynyl, 2-propynyl, 3-butynyl, 2-pentynyl, 1-butynyl and the like. Additional examples or generally applicable substituents are illustrated by the specific compounds described herein.

As used herein, “acyl,” refers to a straight or branched chain hydrocarbon containing from 2 to 30 carbons and at least one carbon of the hydrocarbon chain is substituted with an oxo (═O). In some embodiments, the acyl group contains 2 to 25, 2 to 24, 17 to 20, 2 to 10, 2 to 8 carbon atoms. In one embodiment, the acyl group contains 15, 16, 17, 18, or 19 to 20 carbon atoms. In some embodiments, the acyl group contains 16 or 17 to 20 carbon atoms. In still other embodiments, the acyl group contains 15, 16, 17, 18, 19 or 20 carbon atoms, and in yet other embodiments, the acyl group contains 2-5, 2-4 or 2-3 carbon atoms. Additional examples or generally applicable substituents are illustrated by the specific compounds described herein.

As used herein, the term “alkoxy” refers to an alkyl group, as previously defined, attached to the parent molecular moiety through an oxygen atom. In some embodiments the alkoxy group contains 1-30 carbon atoms. In other embodiment, the alkoxy group contains 1 -20, 1-10 or 1-5 carbon atoms. In some embodiments, the alkoxy group contains 2 to 25, 2 to 24, 15 to 20, 2 to 10, 2 to 8 carbon atoms. In one embodiment, the alkoxy group contains 15, 16, 17, 18 or 19 to 20 carbon atoms. In some embodiments, the alkoxy group contains 15 to 20 carbon atoms. In still other embodiments, the alkoxy group contains 15, 16, 17, 18, 19 or 20 carbon atoms. In some embodiments, the alkoxyl group contains 1 to 8 carbon atoms. In some embodiments, the alkoxyl group contains 1 to 6 carbon atoms. In some embodiments, the alkoxyl group contains 1 to 4 carbon atoms. In still other embodiments, alkoxyl group contains 1-5 carbon atoms, and in yet other embodiments, alkoxyl group contain 1-4 or 1-3 carbon atoms. Representative examples of alkoxyl include, but are not limited to, methoxy, ethoxy, propoxy, isopropoxy, n-butoxy, tert-butoxy, and n-pentoxy. Additional examples or generally applicable substituents are illustrated by the specific compounds described herein.

The term “aliphatic moiety” as used herein, includes saturated, unsaturated, straight chain (i.e., unbranched), or branched, hydrocarbons, which are optionally substituted with one or more functional groups. In some embodiments, the aliphatic may contain one or more function groups selected from double bond, triple bond, carbonyl group (C═O), —O—C(═O)—, —C(—O)—O—, or a combination thereof. As will be appreciated by one of ordinary skill in the art, “aliphatic moiety” is intended herein to include, but is not limited to, alkyl, alkenyl, alkynyl, ester or acyl moieties. Thus, as used herein, the term “alkyl” includes straight, branched saturated groups. An analogous convention applies to other generic terms such as “alkenyl”, “alkynyl”, “acyl” “ester” and the like. Furthermore, as used herein, the terms “alkyl”, “alkenyl”, “alkynyl”, “acyl”, “ester” and the like encompass both substituted and unsubstituted groups. In some embodiments, the term “aliphatic moiety” refers to —(C₁-C₂₄)alkyl, —(C₂-C₂₄)alkenyl, —(C₂-C₂₄)alkynyl, —(C₁-C₂₄)acyl, —C(═O)O—(C₁-C₂₄)alkyl, —O—C(═O)—(C₁-C₂₄)alkyl, —C(—O)O—(C₁-C₂₄)alkenyl, —O—C(═O)—(C₁-C₂₄)alkenyl, —C(═O)O—(C₁-C₂₄)alkynyl, or —O—C(═O)—(C₁-C₂₄)alkynyl. As understood by one skilled in the art, the range of carbon number indicated above encompasses individual number within the range.

As used herein, “cycloalkyl” refers to a monovalent saturated cyclic or bicyclic hydrocarbon group of 3-12 carbons derived from a cycloalkane by the removal of a single hydrogen atom. In some embodiments, cycloalkyl contains 3 to 8 carbon atoms. In some embodiments, cycloalkyl contains 3 to 6 carbon atoms. Cycloalkyl groups may be optionally substituted with alkyl, alkoxy, halo, amino, thiol, or hydroxy substituents. Representative examples of cycloalkyl include, but are not limited to, cyclopropyl, cyclobutyl, cyclopentyl, cyclohexyl, cycloheptyl, and cyclooctyl. Additional examples of generally applicable substituents are illustrated by the specific compounds described herein.

As used herein, “heteroalkyl,” “heteroalkenyl” or “heteroalkynyl” refer to alkyl, alkenyl or alkynyl groups which contain one or more oxygen, sulfur, nitrogen, phosphorus or silicon atoms, e.g., in place of carbon atoms. In some embodiments, the heteroalkyl group contains 1-8 carbon atoms. In certain embodiments, the heteroalkenyl and heteralkynyl groups independently contain 2-8 carbon atoms. In still other embodiments, heteroalkyl, heteroalkenyl and heteralkynyl independently contain 2-5 carbon atoms, and in yet other embodiments, heteroalkyl, heteroalkenyl and heteralkynyl independently contain 2-4 or 2-3 carbon atoms.

The term “heterocycloalkyl” or “heterocycle”, as used herein, refers to a non-aromatic, saturated or unsaturated, 5-, 6- or 7-membered ring or a polycyclic group, including, but not limited to a bi- or tri-cyclic having between one or more heteroatoms independently selected from oxygen, sulfur and nitrogen as part of the ring, wherein (i) each 5-membered ring has 0 to 1 double bonds and each 6-membered ring has 0 to 2 double bonds, (ii) the nitrogen and sulfur heteroatoms may be optionally oxidized, (iii) the nitrogen heteroatom may optionally be quaternized, and/or (iv) any of the above heterocyclic rings may be fused to a benzene ring. Exemplary heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “haloalkyl” refers to a straight or branched chain alkyl group as defined herein containing at least one carbon atom substituted with at least one halo group, halo being as defined herein. In some embodiments, the haloalkyl contains 1 to 30 carbon atoms. In some embodiments, the halkalkyl contains 1 to 8 or 1 to 6 carbon atoms. In other embodiments, the haloalkyl contains 1 to 4 carbon atoms. Additional examples or generally applicable substituents are illustrated by the specific embodiments shown in the Examples which are described herein.

As used herein, the term “aryl” refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated. For example, an aryl may be substituted with one or more heteroatoms (e.g., oxygen, sulfur and/or nitrogen). Additional examples or generally applicable substituents are illustrated by the specific embodiments shown in the Examples which are described herein.

In some embodiments, alkyl, alkenyl, alkynyl, alkoxy, cycloalkyl, cycloalkenyl, heterocycloalkyl, aryl, heteroaryl, acyl, described herein include both substituted and unsubstituted moieties. Exemplary substituents include, but are not limited to, halo, hydroxyl, amino, amide, —SH, cyano, nitro, thioalkyl, carboxylic acid, —NH—C(═NH)—NH₂, alkyl, alkenyl, alkynyl, aryl, heteroaryl, cycloalkyl, heterocycloalkyl, in which alkyl, alkenyl, alkynyl, alkoxyl, aryl, heteroaryl, eycloalkyl, and heterocycloalkyl may be further substituted.

As used herein, the term “amino acid” refers to a compound comprising a primary amino (—NH₂) group and a carboxylic acid (—COOH) group. The amino acids used in the present invention include naturally occurring and synthetic α, β, γ or δ amino acids and L, D amino acids, and include but are not limited to, amino acids found in proteins. Exemplary amino acids include, but are not limited to, glycine, alanine, valine, leucine, isoleucine, methionine, phenylalanine, tryptophan, praline, serine, threonine, cysteine, tyrosine, asparagine, glutamine, aspartate, glutamate, lysine, arginine and histidine. In some embodiments, the amino acid may be a derivative of alanyl, valinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. Additionally, as used herein, “amino acids” also include derivatives of amino acids such as esters, and amides, and salts, as well as other derivatives, including derivatives having pharmacoproperties upon metabolism to an active form.

As used herein, the term “natural a amino acid” refers to a naturally occurring α-amino acid comprising a carbon atom bonded to a primary amino (—NH₂) group, a carboxylic acid (—COOH) group, a side chain, and a hydrogen atom. Exemplary natural a amino acids include, but are not limited to, glycine, alanine, valine, leucine, isoleucine, methionine, phenylalanine, tryptophane, proline, serine, threonine, cysteine, tyrosine, asparaginate, glutaminate, aspartate, glutamate, lysine, arginine and histidine.

Subjects to be treated by the methods of the present invention are, in general, mammalian and primate subjects (e.g., human, monkey, ape, chimpanzee). Subjects may be male or female and may be of any age, including prenatal (i.e., in utero), neonatal, infant, juvenile, adolescent, adult, and geriatric subjects. Thus, in some cases the subjects may be pregnant female subjects. Treatment may be for any purpose, including the therapeutic treatment of previously infected subjects, as well as the prophylactic treatment of uninfected subjects (e.g., subjects identified as being at high risk for infection).

As used herein, “Human immunodeficiency virus” (or “HIV”) as used herein is intended to include all subtypes thereof, including HIV subtypes A, B, C, D, E, F, G, and O, and HIV-2.

As used herein, “Hepatitis B virus” (or “HBV”) as used herein is intended to include all subtypes (adw, adr, ayw, and ayr) and or genotypes (A, B, C, D, E, F, G, and H) thereof.

As used herein, or “a therapeutically effective amount” refers to an amount that will provide some alleviation, mitigation, and/or decrease in at least one clinical symptom in the subject. Those skilled in the art will appreciate that the therapeutic effects need not be complete or curative, as long as some benefit is provided to the subject.

As used herein, “specificity” or “specifically against” refers to a compound that may selectively inhibit the metabolic activity and/or DNA replication of a certain type of virally infected cells. The specificity may be tested by using any methods known to one skilled in the art, for example, testing IC₉₀ and/or IC₅₀. In some embodiments, the compounds described herein may have IC₉₀ and/or IC₅₀ against viral infected cells to be at least about three fold lower than the IC₉₀ and/or IC₅₀ against normal (uninfected) cells. In some embodiments, the compounds described herein may have IC₉₀ and/or IC₅₀ against viral infected cells to be about three fold to ten fold lower than the IC₉₀ and/or IC₅₀ against normal (uninfected) cells. In some embodiments, the compounds described herein may have IC₉₀ and/or IC₅₀ against viral infected cells to be at least ten fold lower than the IC₉₀ and/or IC₅₀ against normal (uninfected) cells. In some embodiments, the compounds described herein may have specific cytotoxicity against viral infected and/or transformed cells. The cytotoxicity may be measured by any methods known to one skilled in the art.

Unless otherwise stated, structures depicted herein are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the terms “treatment,” “treat,” and “treating” refer to reversing, alleviating, inhibiting the progress of a disease or disorder as described herein, or delaying, eliminating or reducing the incidence or onset of a disorder or disease as described herein, as compared to that which would occur in the absence of the measure taken. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Active compounds of the present invention may optionally be administered in combination (or in conjunction) with other active compounds and/or agents useful in the treatment of viral infections as described herein. The administration of two or more compounds “in combination” or “in conjunction” means that the two compounds are administered closely enough in time to have a combined effect, for example an additive and/or synergistic effect. The two compounds may be administered simultaneously (concurrently) or sequentially or it may be two or more events occurring within a short time period before or after each other. Simultaneous administration may be carried out by mixing the compounds prior to administration, or by administering the compounds at the same point in time but at different anatomic sites or using different routes of administration. In some embodiments, the other antiviral agent may optionally be administered concurrently.

“Parenteral” as used herein refers to subcutaneous, intravenous, intra-arterial, intramuscular or intravitreal injection, or infusion techniques.

“Topically” as used herein encompasses administration rectally and by inhalation spray, as well as the more common routes of the skin and mucous membranes of the mouth and nose and in toothpaste.

### B. COMPOUNDS

According to some aspects of the present invention, compounds with a range of biological properties are provided. Compounds described herein have biological activities relevant for the treatment of diseases associated with at least one virus.

(1) According to one aspect of the present invention, the compounds have the structure of Formula A, A′, B or B′

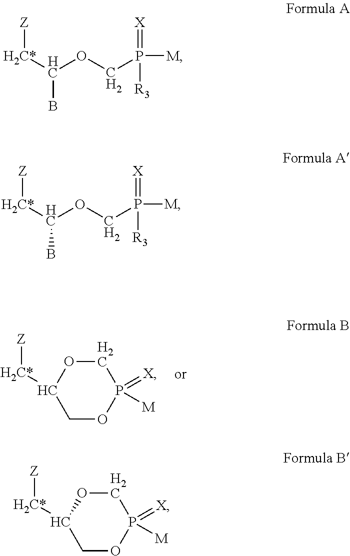

wherein:

M is

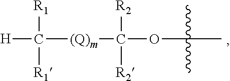

and the oxygen of M is bonded to —P(═X)(R₃)—,

Q, when present, is:

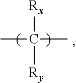

R₁, R₁′, R₂, R₂′, Rₓ and R_{y} are independently —H, halogen, —ORⁱ, —SRⁱ, —NHRⁱ, or NRⁱRⁱⁱ,

and Rⁱ and Rⁱⁱ are independently hydrogen or an aliphatic moiety,

and m is an integer from 0 to 6,

B is selected from the group consisting of hydrogen, F, CF₃, CHF₂, —CH₃, —CH₂CH₃, —CH₂OH, —CH₂CH₂OH, —CH(OH)CH₃, —CH₂F, —CH═CH₂, and —CH₂N₃,

X is selenium, sulphur, or oxygen (in some embodiments, X is oxygen);

R₃ is hydroxy, —OR₂ₐ, C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, or —NR′R″ (in some embodiments, R₃ is hydroxyl)
R₂ₐ is C₁₋₈ alkyl, C₂₋₈ alkenyl, C₂₋₈ alkynyl, C₁₋₈ heteroalkyl, C₂₋₈ heteroalkenyl, C₂₋₈ heteroalkynyl, —P(═O)(OH)₂, or —P(═O)(OH)OP(═O)(OH)₂,
R′ and R″are independently selected from the group consisting of H, C₁₋₈ alkyl, C₂₋₈ alkenyl, C₂₋₈ alkynyl, C₁₋₈ heteroalkyl, C₂₋₈ heteroalkynyl, C₂₋₈ heteroalkenyl, and C₆₋₁₀ aryl, or
NR′R″ is a substituted or unsubstituted amino acid residue;

Z comprising a heterocyclic moiety comprising at least one N (in some embodiments, the heterocyclic moiety is purine or pyrimidine), and

the symbol * indicates the point of attachment of the methylene moiety in Formula A, A′, B or B′ to Z is via an available nitrogen of the heterocyclic moiety,

or a pharmaceutically acceptable salt thereof.

In some embodiments, the compound is in the form of an enantiomer, diastereomer, racemate, stereoisomer, tautomer, rotamer or a mixture thereof.

In another embodiment, B is —CH₃ or —CH₂OH.

In some embodiments, R₃ is hydroxyl.

In some embodiments, M is selected from —O—(CH₂)₂—O—C₁₋₂₄alky, —O—(CH₂)₃—O—C₁₋₂₄ alkyl, —O—CH₂—CH(OH)—CH₂—O—C₁₋₂₄alkyl, and —O—CH₂—CH(OH)—CH₂—S—C₁₋₂₄alkyl. In another embodiment, M is —O—(CH₂)ₐ—O—(CH₂)ₜ—CH₃, wherein a is 2 to 4 and t is 11 to 19. In some embodiments, a is 2 or 3 and t is 15 or 17. In some embodiments, M is —O—(CH₂)₂—O—(CH₂)₁₅CH₃ or —O—(CH₂)₂—O—(CH₂)₁₇C14₃. In one embodiment, M is —O—(CH₂)₃—O—(CH₂)₁₅CH₃ or —O—(CH₂)₃—O—(CH₂)₁₇CH₃.

In one embodiment, the compound has the structure of Formula C:

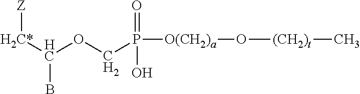

wherein:
a is 2 to 4, (in one embodiment, a is 2 or 3)
t is 11 to 19 (in one embodiment, t is 15, 16 or 17) and
B is hydrogen, —CH₃, or —CH₂OH (in one embodiment, B is —CH₃),

or a pharmaceutically acceptable salt thereof.

In one embodiment, M is selected from formula a, b or c.

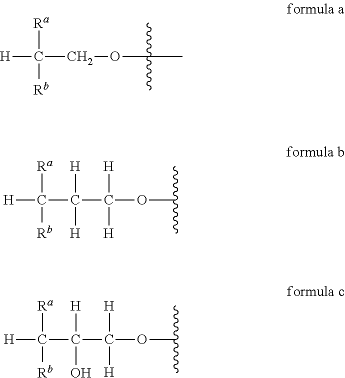

wherein R^{a} and R^{b} are independently —H, halogen, —ORⁱ, —SRⁱ, NHRⁱ, or —NRⁱRⁱⁱ, and Rⁱ and Rⁱⁱ are independently hydrogen or an aliphatic moiety. In some embodiments, Rⁱ and Rⁱⁱ are independently —(C₁-C₂₄)alkyl, —(C₂-C₂₄)alkenyl, —(C₂-C₂₄)alkynyl or —(C₁-C₂₄)acyl.

In some embodiments, at least one of R^{a} or R^{b} is not hydrogen. In some embodiments, R^{a} and R^{b} are independently selected from the group consisting of —H, optionally substituted —O(C₁-C₂₄)alkyl, —O(C₂-C₂₄)alkenyl, —O(C₁-C₂₄)acyl, —S(C₂-C₂₄)-alkyl, —S(C₂-C₂₄)alkenyl, and —S(C₁-C₂₄)acyl.

In some embodiments, for M, R₁, R₁′, R₂, R₂′, Rₓ and R_{y} are independently selected from —O(C₁-C24)alkyl, —O(C₂-C₂₄)alkenyl, —O(C₂-C₂₄)alkynyl, —O(C₁-C₂₄)acyl, —S(C₁-C₂₄)alkyl, —S(C₂-C₂₄)alkenyl, —S(C₂-C₂₄)alkynyl, —S(C₁-C₂₄)acyl, —NH(C₁-C₂₄)alkyl, —NH(C₂-C₂₄)alkenyl, —NH(C₂-C₂₄)alkynyl, —NH(C₁-C₂₄)acyl, —N((C₁-C₂₄)alkyl)((C₂-C₂₄)alkyl), —N((C₁-C₂₄)alkyl)((C₂-C₂₄)alkenyl), —N((C₁-C₂₄)alkyl)((C₂-C₂₄)acyl), —N((C₁-C₂₄)alkyl)((C₂-C₂₄)alkynyl), —N((C₂-C₂₄)alkeyl)((C₂-C₂₄)alkynyl), —N((C₂-C₂₄)alkenyl)((C₂-C₂₄)alkenyl), —N((C₂-C₂₄)alkynyl)((C₂-C₂₄)alkynyl), —N((C₁-C₂₄)acyl)((C₂-C₂₄)alkynyl), or —N((C₁-C₂₄)acyl)((C₂-C₂₄)alkenyl).

In one embodiment, Z comprises (or is) purine or pyrimidine, which may be optionally substituted by at least one substituent. In some embodiments, at least one substituent may be selected from the group consisting of halogen, hydroxyl, amino, substituted amino, di-substituted amino, sulfur, nitro, cyano, acetyl, acyl, aza, C₁₋₆ alkyl, C₂₋₆ alkenyl, C₂₋₆ alkynyl, C₆₋₁₀ aryl, and carbonyl substituted with a C₁₋₆ alkyl, C₂₋₆ alkenyl, C₂₋₆ alkynyl, or C₆₋₁₀ aryl, haloalkyl and aminoalkyl.

In some embodiments, Z may be selected from adenine, 6-chloropurine, xanthine, hypoxanthine, guanine, 8-bromoguanine, 8-chloroguanine, 8-aminoguanine, 8-hydrazinoguanine, 8-hydroxyguanine, 8-methylguanine, 8-thioguanine, 2-aminopurine, 2,6-diaminopurine, thymine, cytosine, 5-fluorocytosine, uracil; 5-bromouracil, 5-iodouracil, 5-ethyluracil, 5-ethynyluracil, 5-propynyluracil, 5-propyluracil, 5-vinyluracil, or 5-bromovinyluracil. In some embodiments, Z is selected from guanin-9-yl, adenin-9-yl, 2, 6-diaminopurin-9-yl, 2-aminopurin-9-yl or their 1-deaza, 3-deaza, 8-aza compounds, or cytosin-1-yl. In some embodiments, Z is guanin-9-yl or 2, 6-diaminopurin-9-yl.

In another embodiment, Z is selected from 6-alkylpurine and N⁶-alkylpurines, N⁶-acylpurines, N⁶-benzylpurine, 6-halopurine, N⁶-acetylenic purine, N⁶-acyl purine, N⁶-hydroxyalkyl purine, 6-thioalkyl purine, N²-alkylpurines, N⁴-alkylpyrimidines, N⁴-acylpyrimidines, 4-halopyrimidines, N⁴-acetylenic pyrimidines, 4-amino and N⁴-acyl pyrimidines, 4-hydroxyalkyl pyrimidines, 4-thioalkyl pyrimidines, thymine, cytosine, 6-azapyrimidine, including 6-azacytosine, 2- and/or 4-mereaptopyrimidine, uracil, C⁵-alkylpyrimidines, C⁵-benzylpyrimidines, C⁵-halopyrimidines, C⁵-vinylpyrirnidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, acyl groups such as acetyl and propionyl, methanesulfonyl, and p-toluenesulfonyl. Preferred bases include cytosine, 5-fluorocytosine, uracil, thymine, adenine, guanine, xanthine, 2,6-diaminopurine, 6-aminopurine, 6-chloropurine and 2,6-dichloropurine.

In one embodiment, Z is

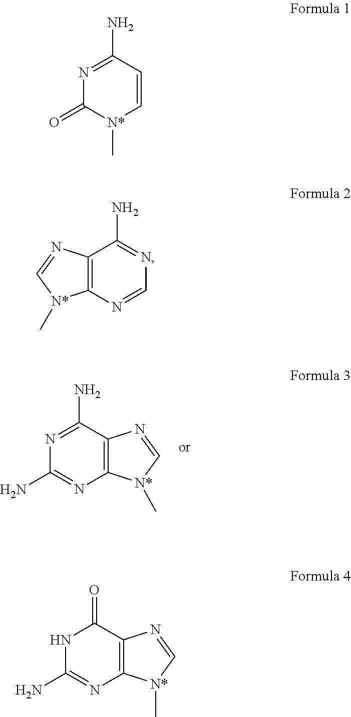

wherein the symbol * in Formula 1-4 indicates the point of attachment of N to the methylene in Formula A, A′, B or B′.

The example of Z is further described in U.S. Pat. No. 6,583,149, which is incorporated by reference in its entirety.

Additional examples of Z include, but are not limited to, moieties of the general formula:

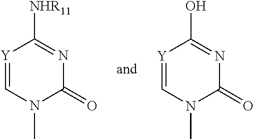

where:

Y is N or CX;

X is selected from the group consisting of H, halo, C₁₋₆ alkyl, C₂₋₆ alkenyl, C₂₋₆ alkynyl, CN, CF₃, N₃, NO₂, C₆₋₁₀) aryl, C₆₋₁₀ heteroaryl, and COR_{b};
R_{b} is selected from the group consisting of H, OH, SH, C₁₋₆ alkyl, C₁₋₆ aminoalkyl,

C₁₋₆ alkoxy and C₁₋₆ thioalkyl; and

R₁₁ is selected from the group consisting of H, C₁₋₆ alkyl, C₂₋₆ alkenyl, C₂₋₆ alkynyl, C₆₋₁₀ aryl, C₃₋₁₀cycloalkyl and carbonyl substituted with a C₁₋₆ alkyl, C₂₋₆ alkenyl, C₂₋₆ alkynyl, or C₆₋₁₀ aryl.

Additional examples of Z include, but are not limited to, compounds of the general formula:

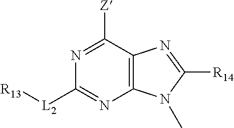

where:

Z′ is —NRₐR_{b}, —SRₐ or —ORₐ,

L₂ is a covalent bond, or is —N(-R₁₅)—, N(—R₁₅)C(═O)-, -0-, -S-, -S(═O)-, or is —S(═O)₂—,

R₁₃ is H, C₁₋₆ alkyl, C₁₋₆ heteroalkyl, C₂₋₆ alkenyl, C₆₋₁₀ aryl, C₇₋₁₆ arylalkyl, C₃₋₁₀ carbocyclyl, C₆₋₁₀ heterocyclyl, or C₇₋₁₆ heterocyclylalkyl;

R₁₄ is H, halo, hydroxy, alkoxy, —O(CH₂)ₓOC(═O)OR₁₅, or OC(═O)0R₁₅, wherein x is 2 or 3 to 10, 15 or 20, or wherein each occurrence of Rᵢ and Rᵢᵢ are independently selected from the group consisting of hydrogen, C₁₋₆ alkyl, C₂₋₆ alkenyl, C₂₋₆ alkynyl, C₃₋₆ cycloalkyl, and C₃₋₈ heterocyclyl; and

R₁₅ is H, C₁₋₆ alkyl, C₁₋₆ heteroalkyl, C₂₋₆ alkenyl, C₆₋₁₀ aryl, C₇₋₁₆ arylalkyl, C₃₋₁₀ cycloalkyl, C₆₋₁₀ heterocyclyl, or C₇₋₁₆ heterocyclalkyl

Rₐ, R_{b} are independently selected from the group consisting of hydrogen, C₁₋₆ alkyl, C₁₋₆acyl, or C₃₋₆ cycloalkyl, and C₃₋₈ heterocyclyl, wherein C₃₋₆ cycloalkyl and C₃₋₈ heterocyclyl may be optionally substituted with one or more C₁₋₅ alkyl.

Additional examples of Z include, but are not limited to, moiety of the general formula:

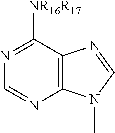

R₁₆ and R₁₇ are independently selected from the group consisting of hydrogen, C₁₋₆ alkyl, C₁₋₆ acyl or C₃₋₆ cycloalkyl, or C₃₋₈ heterocyclyl, wherein C₃₋₆ cycloalkyl and C₃₋₈ heterocyclyl can be optionally substituted with one or more C₁₋₅ alkyl.

The exemplary compounds of the present invention include, but are not limited to,

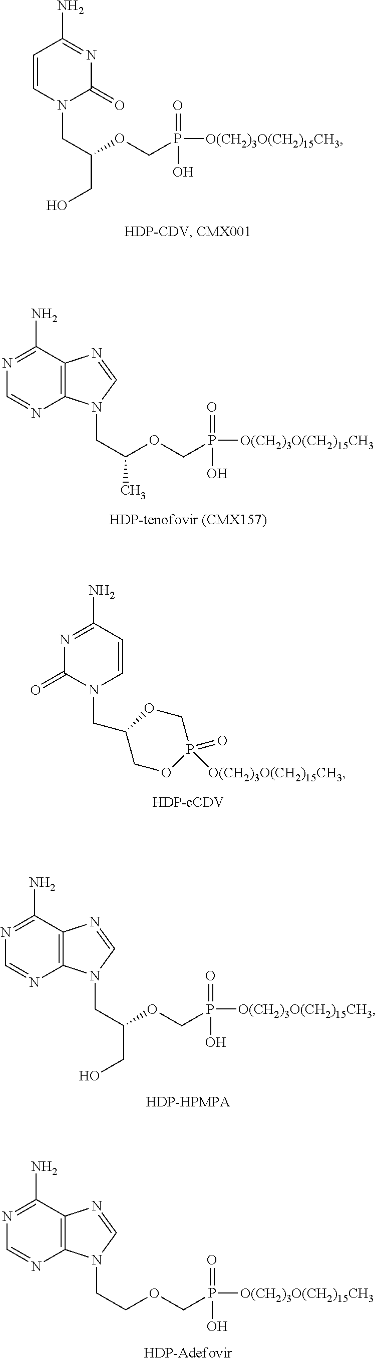

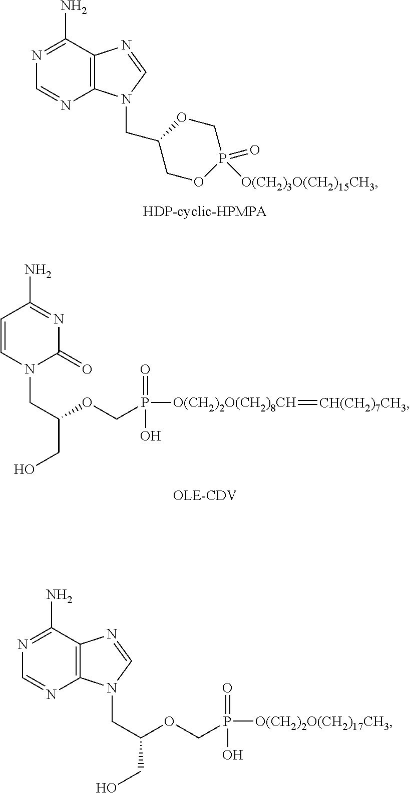

or a pharmaceutically acceptable salt thereof.

More exemplary compounds are shown below:

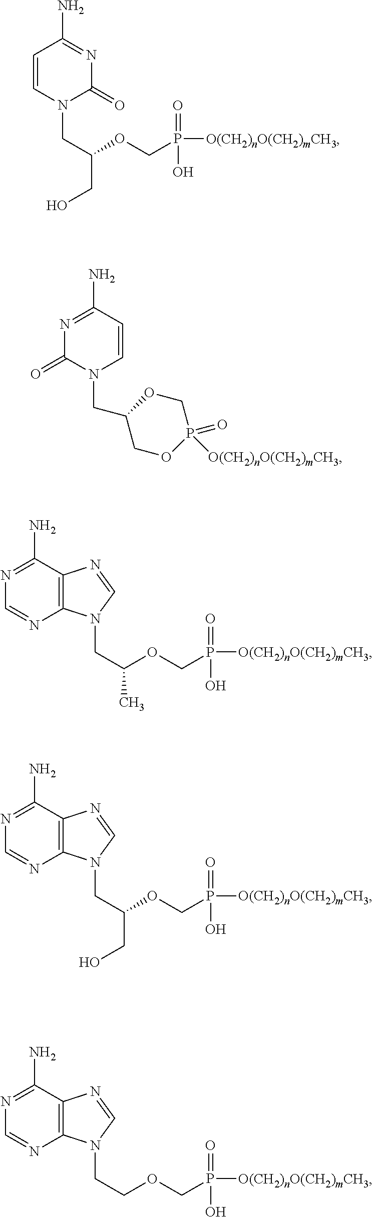

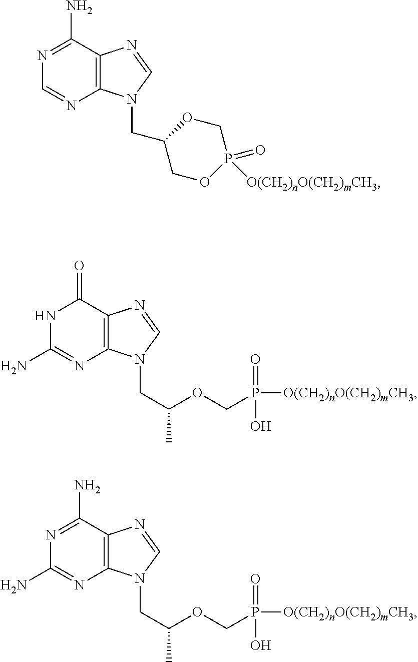

wherein each occurrence of n is independently 2 or 3, and each occurrence of m is independently 15, 16 or 17.

(2) According to some aspects of the present invention, the compounds of the present invention have the structure of Formula I:

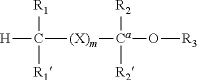

wherein:

R₁, R₁′, R₂ and R₂′ are independently —H, halogen, —ORⁱ, —SRⁱ, —NHRⁱ, —NRⁱRⁱⁱ, and Rⁱ

and Rⁱⁱ are independently hydrogen or aliphatic, and

R₃ is a pharmaceutically active phosphonate, bisphosphonate or a phosphonate derivative of a pharmacologically active compound;

X, when present, is:

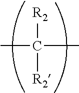

and m is an integer from 0 to 6.

In some embodiments, said alkyl, alkenyl, alkynyl or acyl moieties optionally have 1 to 6 double bonds.

In some embodiments, at least one of R₁ and R₁′ are not —H.

In some embodiments, m is 0, 1 or 2. In one embodiment, R₂ and R₂′ are H. In another embodiment, the compounds are ethanediol, propanediol or butanediol derivatives of a therapeutic phosphonate. In one embodiment, the compounds of the present invention are ethanediol phosphonate species has the structure:

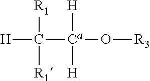

wherein R₁, R₁′, and R₃ are as defined above.

In some embodiments, the compounds of the present invention are propanediol species that have the structure:

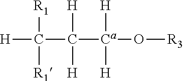

wherein m is 1 and R₁, R₁′, and R₃ are as defined above in the general formula.

In one embodiment, the compounds of the present invention are glycerol species that have the structure:

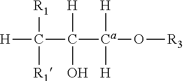

wherein m is 1, R₂ is H, R₂′ is OH, and R₂ and R₂′ on C^{α} are both —H. Glycerol is an optically active molecule. Using the stereospecific numbering convention for glycerol, the sn-3 position is the position which is phosphorylated by glycerol kinase. In compounds of the invention having a glycerol residue, the R₃ moiety may be joined at either the sn-3 or sn-1 position of glycerol.

In some embodiments, R₁ is an alkoxy group having the formula —O—(CH₂)ₜ—CH₃, wherein t is 0-24. In one embodiment, t is 11-19. In another embodiment, t is 15 or 17.

Additionally, antiviral phosphonates such as cidofovir, cyclic-eidofovir, adefovir, tenofovir, and the like, may be used as an R₃ group in accordance with the present invention.

(3) Compounds, compositions, formulations, and methods of treating subjects that can be used to carry out the present invention include, but are not limited to, those described in U.S. Pat. No. 6,716,825, 7,034,014, 7,094,772, 7,098,197, and 7,452,898, and 7,687,480 the disclosures of which are incorporated by reference herein in their entireties.

In some embodiments, the active compounds have the structure Formula C:

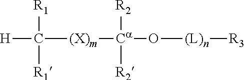

wherein:

R₁, R₁′, R₂ and R₂′ are independently —H, oxo, halogen, —NH₂, —OH, or —SH or optionally substituted —XRⁱ, and wherein X is O, S, —NH, or —NRⁱⁱ, and Rⁱ and Rⁱⁱ are independently —(C₁-C₂₄)alkyl, —(C₁-C₂₄)alkenyl, —(C₁-C₂₄)alkynyl, or —(C₁-C₂₄)acyl.

In some embodiments, at least one of R₁ and R₁′ are not —H. In some embodiments, said alkenyl or acyl moieties optionally have 1 to 6 double bonds,

R₃ is a pharmaceutically active phosphonate, bisphosphonate or a phosphonate derivative of a pharmacologically active compound, linked to a functional group on optional linker L or to an available oxygen atom on C^{α};

X, when present, is:

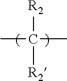

L is a valence bond or a bifunctional linking molecule of the formula -J-(CR₂)ₜ-G-, wherein t is an integer from 1 to 24, J and G are independently —O—, —S—, —C(O)O—, or —NH—, and R is —H, substituted or unsubstituted alkyl, or alkenyl;

m is an integer from 0 to 6; and

n is 0 or 1.

In some embodiments, m=0, 1 or 2. In some embodiments, R₂ and R₂′ are H, and the prodrugs are then ethanediol, propanediol or butanediol derivatives of a therapeutic phosphonate. A exemplary ethanediol phosphonate species has the structure:

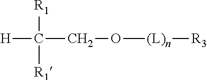

wherein R₁, R₁′, R₃, L, and n are as defined above.

In some embodiments, propanediol species has the structure:

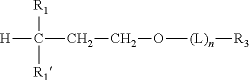

wherein m=1 and R₁, R₁′, R₃, L and n are as defined above in the general formula.

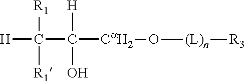

wherein m=1, R₂═H, R₂′═OH, and R₂ and R₂′ on C^{α} are both —H. Glycerol is an optically active molecule. Using the stereospecific numbering convention for glycerol, the sn-3 position is the position which is phosphorylated by glycerol kinase. In compounds of the invention having a glycerol residue, the -(L)ₙ-R₃ moiety may be joined at either the sn-3 or sn-1 position of glycerol.

In another embodiment, R₁ is an alkoxy group having the formula —O—(CH₂)ₜ—CH₃, wherein t is 0-24. More preferably t is 11-19. Most preferably t is 15 or 17.

Exemplary R₃ groups include bisphosphonates that are known to be clinically useful, for example, the compounds:

Etidronate: 1-hydroxyethylidene bisphosphonic acid (EDHP);

Clodronate: dichloromethylene bisphosphonic acid (Cl₂ MDP);

Tiludrom ate: chloro-4-phenylth ° methylene bisphosphonic acid;

Pamidronate: 3-amino-1-hydroxypropylidene bisphosphonic acid (ADP);

Alendronate: 4-amino-1-hydroxybutylidene bisphosphonic acid;

Olpadronate: 3 dimethyl amino-1-hydroxypropylidene bisphosphonic acid (dimethyl-APD);

Ibandronate: 3-methylpentylamino-1-hydroxypropylidene bisphosphonic acid (BM 21.0955);

EB-1053 : 3-(1-pyrrolidinyl)-1-hydroxypropylidene bisphosphonic acid;

Risedmnate: 2-(3-pyridinyl)-1-hydroxy-ethylidene bisphosphonic acid;

Amino-Olpadronate: 3 -(N,N-diimethylanino-1-aminopropylidene)bisphosphonate (IG9402), and the like.

R₃ may also be selected from a variety of phosphonate-containing nucleotides (or nucleosides which can be derivatized to their corresponding phosphonates), which are also contemplated for use herein. Preferred nucleosides include those useful for treating disorders caused by inappropriate cell proliferation such as 2-chloro-deoxyadenosine, 1-β-D-arabinofuranosyl-cytidine (cytarabine, ara-C), fluorouridine, fluorodeoxyuridine (floxuridine), gemcitabine, cladribine, fludarabine, pentostatin (2′-deoxycoformycin), 6-mercaptopurine, 6-thioguanine, and substituted or unsubstituted 1-β-D-arabinofuranosyl-guanine (ara-G), 1-β-D-arabinofuranosyl-adenosine (ara-A), 1-β-D-arabinofuranosyl-uridine (ara-U), and the like.

Nucleosides useful for treating viral infections may also be converted to their corresponding 5′-phosphonates for use as an R₃ group. Such phosphonate analogs typically contain either a phosphonate (—PO₃H₂) or a methylene phosphonate (—CH₂—PO₃H₂) group substituted for the 5′-hydroxyl of an antiviral nucleoside. Some examples of antiviral phosphonates derived by substituting —PO₃H₂ for the 5′-hydroxyl are:

| | | |
|---|---|---|
| 3′-azido-3′,5′- dideoxythymidine-5′- phosphonic acid (AZT phosphonate) | 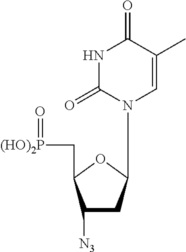 | Hakimelahi, G. H.; Moosavi- Movahedi, A. A.; Sadeghi, M. M.; Tsay, S-C; Hwu, J. R. J. Med. Chem. 1995, 38: 4648- 4659. |
| 3′,5′-dideoxythymidine- 2′-ene-5′-phosphonic acid (d4T phosphonate) | 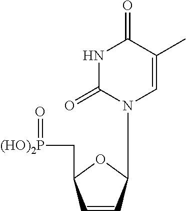 | Hakimelahi, G. H.; Moosavi- Movahedi, A. A.; Sadeghi, M. M.; Tsay, S-C; Hwu, J. R. J. Med. Chem. 1995, 38: 4648- 4659. |
| 2′,3′,5′-trideoxycytidine- 5′-phosphonic acid (ddC phosphonate) | 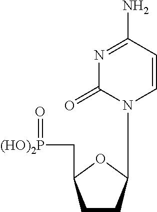 | Kofoed, T., Ismail, A. E. A. A.; Pedersen, E. B.; Nielsen, C. Bull. Soc. Chim. Fr. 1997, 134: 59-65. |
| 9-[3-(phosphono- methoxy)propyl]adenine (Adefovir) | 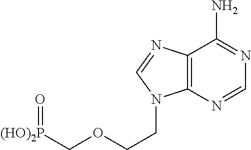 | Kim, C. U.; Luh, B. Y.; Misco, P. F.; Bronson, J. J.; Hitchcock, M. J. M.; Ghazzouli, I.; Martin, J. C. J. Med. Chem. 1990, 33: 1207-1213. |

Some examples of antiviral phosphonates derived by substituting —CH₂—PO₃H₂ for the 5′-hydroxyl are:

| | | |
|---|---|---|
| Ganciclovir phosphonate | 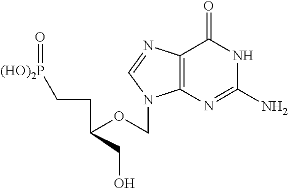 | Huffman, J. H.; Sidwell, R. W.; Morrison, A. G.; Coombs, J., Reist, E. J. Nucleoside Nucleotides, 1994, 13: 607-613. |
| Acyclovir phosphonate | 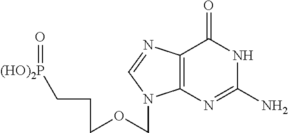 | Huffman, J. H.; Sidwell, R. W.; Morrison, A. G.; Coombs, J., Reist, E. J. Nucleoside Nucleotides, 1994, 13: 607-613. |
| Ganciclovir cyclic phosphonate | 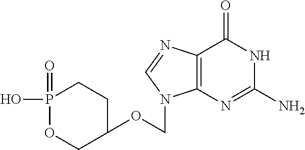 [SMILES: Nc1nc2c(ncn2COC2CCP(=O)(O)OC2)c(=O)[nH]1] | Smee, D. F.; Reist, E. J. Antimicrob. Agents Chemother. 1996, 40: 1964-1966. |
| 3′-thia-2′,3′- dideoxycytidine-5′- phosphonic acid | 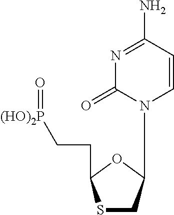 | Kraus, J. L.; Nucleosides Nucleotides, 1993, 12: 157-162. |

Other exemplary antiviral nucleotide phosphonates are derived similarly from antiviral nucleosides including ddA, ddI, ddG, L-FMAU, DXG, DAPD, L-dA, L-dI, L-(d)T, L-dC, L-dG, FTC, penciclovir, and the like.

Additionally, antiviral phosphonates such as cidofovir, cyclic cidofovir, adefovir, tenofovir, and the like, may be used as an R₃ group in accordance with the present invention.

Many phosphonate compounds exist that can be derivatized according to the invention to improve their pharmacologic activity, or to increase their oral absorption, such as, for example, the compounds disclosed in the following patents, each of which are hereby incorporated by reference in their entirety: U.S. Pat. No. 3,468,935 (Etidronate), U.S. Pat. No. 4,327,039 (Pamidronate), U.S. Pat. No. 4,705,651 (Alendronate), U.S. Pat. No. 4,870,063 (Bisphosphonic acid derivatives), U.S. Pat. No. 4,927,814 (Diphosphonates), U.S. Pat. No. 5,043,437 (Phosphonates of azidodideoxynucleosides), U.S. Pat. No. 5,047,533 (Acyclic purine phosphonate nucleotide analogs), U.S. Pat. No. 5,142,051 (N-Phosphonylmethoxyalkyl derivatives of pyrimidine and purine bases), U.S. Pat. No. 5,183,815 (Bone acting agents), U.S. Pat. No. 5,196,409 (Bisphosphonates), U.S. Pat. No. 5,247,085 (Antiviral purine compounds), U.S. Pat. No. 5,300,671 (Gem-diphosphonic acids), U.S. Pat. No. 5,300,687 (Trifluoromethylbenzylphosphonates), U.S. Pat, No. 5,312,954 (Bis- and tetrakis-phosphonates), U.S. Pat. No. 5,395,826 (Guanidinealkyl-1,1-bisphosphonic acid derivatives), U.S. Pat. No. 5,428,181 (Bisphosponate derivatives), U.S. Pat. No. 5,442,101 (Methylenebisphosphonic acid derivatives), U.S. Pat. No. 5,532,226 (Trifluoromethybenzylphosphonates), U.S. Pat. No. 5,656,745 (Nucleotide analogs), U.S. Pat. No. 5,672,697 (Nucleoside-5′-methylene phosphonates), U.S. Pat. No. 5,717,095 (Nucleotide analogs), U.S. Pat. No. 5,760,013 (Thymidylate analogs), U.S. Pat. No. 5,798,340 (Nucleotide analogs), U.S. Pat. No. 5,840,716 (Phosphonate nucleotide compounds), U.S. Pat. No. 5,856,314 (Thio-substituted, nitrogen-containing, heterocyclic phosphonate compounds), U.S. Pat. No. 5,885,973 (olpadronate), U.S. Pat. No. 5,886,179 (Nucleotide analogs), U.S. Pat. No. 5,877,166 (Enantiomerically pure 2-aminopurine phosphonate nucleotide analogs), U.S. Pat. No. 5,922,695 (Antiviral phosphonomethoxy nucleotide analogs), U.S. Pat. No. 5,922,696 (Ethylenic and allenic phosphonate derivatives of purines), U.S. Pat. No. 5,977,089 (Antiviral phosphonomethoxy nucleotide analogs), U.S. Pat. No. 6,043,230 (Antiviral phosphonomethoxy nucleotide analogs), U.S. Pat. No. 6,069,249 (Antiviral phosphonomethoxy nucleotide analogs); Belgium Patent No. 672205 (Clodronate); European Patent No. 753523 (Amino-substituted bisphosphonic acids); European Patent Application 186405 (geminal diphosphonates); and the like.

Certain bisphosphonate compounds have the ability to inhibit squalene synthase and to reduce serum cholesterol levels in mammals, including man. Examples of these bisphosphonates are disclosed, for example, in U.S. Pat. Nos. 5,441,946 and 5,563,128 to Pauls et al. Phosphonate derivatives of lipophilic amines, both of which are hereby incorporated by reference in their entirety. Analogs of these squalene synthase inhibiting compounds according to the invention, and their use in the treatment of lipid disorders in humans are within the scope of the present invention. Bisphosphonates of the invention may be used orally or topically to treat periodontal disease as disclosed in U.S. Pat. No. 5,270,365, hereby incorporated by reference in its entirety.

In some embodiments, the active compounds have a phosphonate ester formed by a covalent linking of an antiviral compound selected from the group consisting of cidofovir, adefovir, cyclic cidofovir and tenofovir, to an alcohol selected from the group consisting of an alkylglycerol, alkylpropanediol, 1-S-alkylthioglycerol, alkoxyalkanol or alkylethanediol, or a pharmaceutically acceptable salt thereof.

In some embodiments, the active compounds comprise an antiviral nucleoside compound, wherein the 5′-hydroxyl group has been substituted for a phosphonate or methyl phosphonate that is covalently linked to an alkylethanediol.

Certain compounds of the invention possess one or more chiral centers, e.g. in the acyclic moieties, and may thus exist in optically active forms. Likewise, when the compounds contain an alkenyl group or an unsaturated alkyl or acyl moiety there exists the possibility of cis- and trans-isomeric forms of the compounds. Additional asymmetric carbon atoms can be present in a substituent group such as an alkyl group. The R- and S-isomers and mixtures thereof, including racemic mixtures as well as mixtures of cis- and trans-isomers are contemplated by this invention. All such isomers as well as mixtures thereof are intended to be included in the invention. If a particular stereoisomer is desired, it can be prepared by methods well known in the art by using stereospecific reactions with starting materials that contain the asymmetric centers and are already resolved or, alternatively, by methods that lead to mixtures of the stereoisomers and resolution by known methods.

In cases where compounds are sufficiently basic or acidic to form stable nontoxic acid or base salts, administration of the compound as a pharmaceutically acceptable salt may be appropriate. Pharmaceutically acceptable salts are salts that retain the desired biological activity of the parent compound and do not impart undesired toxicological effects. Pharmaceutically acceptable salts include those derived from pharmaceutically acceptable inorganic or organic bases and acids. Suitable salts include those derived from alkali metals such as potassium, lithium or sodium; alkaline earth metals such as calcium and magnesium; or any pharmaceutically acceptable amine salts such as a moiety containing an amino group include, for example, ammonium, mono, di, tri or tetra substituted amino groups, or any applicable organic bases containing at least one nitrogen, for example, aniline, indole, piperidine, pyridine, pyrimidine, pyrrolidine.

In some embodiments, the pharmaceutically acceptable salts are selected from organic acid addition salts formed with acids, which form a physiological acceptable anion, for example, acetate, adipate, aspartate, benzoate, besylate, bicarbonate/carbonate, bisulphate/sulphate, borate, camsylate, citrate, cyclamate, bicarbonate, carbonate, disylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts.

Exemplary agent that may be used to form the salt include, but are not limited to, (1) acids such as inorganic acid, for example, hydrochloric acid, hydrobromic acid, sulfuric acid, phosphoric acid, nitric acid; or organic acids, for example, acetic acid, citric acid, fumaric acid, alginic acid, gluconic acid, gentisic acid, hippuric acid, benzoic acid, maleic acid, tannic acid, L-mandelic acid, orotic acid, oxalic acid, saccharin, succinic acid, L-tartaric acid, ascorbic acid, palmitic acid, polyglutamie acid, toluenesulfonic acid, naphthalenesulfonic acid, methanesulfonic acid, naphthalenedisulfonic acid, polygalacturonic acid, (2) bases such as ammonia, mono, di, tri or tetra-substituted ammonia, alkali metal bases such as potassium hydroxide, lithium hydroxide, sodium hydroxide; alkaline earth bases such as magnesium hydroxide, calcium hydroxide; organic bases such as L-arginine, diethylamine, diethylaminoethanol, dicyclohexylamine, ethylenediamine, imidazole, L-lysine, 2-hydroxyethylmorpholine, N-methyl-glucamine, potassium methanolate, zinc tert-butoxide.

One aspect of the invention provides compounds of Formula D

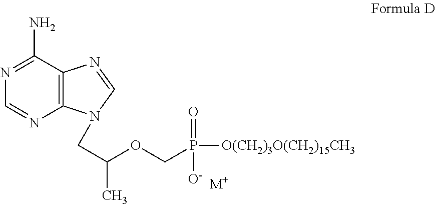

wherein M⁺ is potassium (K⁺), sodium (Na⁺), lithium (Li⁺), calcium (Ca²⁺), magnesium (Mg₂₊), or any pharmaceutically acceptable cation containing at least one nitrogen. Exemplary cations containing at least one nitrogen include, but are not limited to, various ammonium, mono, di, tri or tetra substituted amino cations. In one embodiment, the cations containing at least one nitrogen may be represented by the formula of [NR₁R₂R₃R₄]⁺ and R₁, R₂, R₃, and R₄ are independently hydrogen or aliphatic moiety. In one embodiment, the aliphatic moiety is selected from C₁₋₅ alkyl (e.g., NH₄⁺, NH₃CH₃⁺, N H₃CH₂CH₃⁺, etc.), C₁₋₅ alkenyl, or C₁₋₅ alkynyl, etc. In some embodiments, M⁺ is potassium (K⁺), sodium (Na⁺), or lithium (Li⁺). In one embodiment, M⁺ is K⁺. For compounds of formula I, when M⁺ is a cation with multiple charges, multiple equivalents of anions will present to meet the cation-anion balance. For example, when the cation is Ca²⁺ or Mg²⁺, two equivalents of the anions are present to meet the requirement for cation-anion balance.

In one embodiment, the compound has the structure of

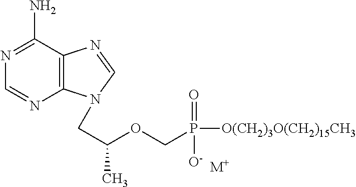

The salt may be in various forms, all of which are included within the scope of the invention. These forms include anhydrous form or solvates. In one embodiment, M⁺ is K⁺, Na⁺, or Li⁺. In other embodiments, the salt may be in the crystalline form with various degrees. In one embodiment, the compound is in an anhydrous form, a solvate or crystalline form.

Active compounds as described herein can be prepared in accordance with known procedures, or variations thereof that will be apparent to those skilled in the art. See, e.g., Painter et al., Evaluation of Hexadecyloxypropyl-9-R-[2-(Phosphonomethoxy)Propyl]-Adenine, CMX157, as a Potential Treatment for Human Immunodeficiency Virus Type 1 and Hepatitis B Virus Infections, Antimicrobial Agents and Chemotherapy 51, 3505-3509 (2007) and US Patent Application Publication No. 2007/0003516 to Almond et al.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### B. SYNTHESIS OF ACTIVE COMPOUNDS

The process to be utilized in the preparation of the compounds described herein depends upon the specific compound desired. Such factors as the selection of the specific substituent and various possible locations of the specific substituent all play a role in the path to be followed in the preparation of the specific compounds of this invention. Those factors are readily recognized by one of ordinary skill in the art.

In general, the compounds of this invention may he prepared by standard techniques known in the art and by known processes analogous thereto. General methods for preparing compounds of the present invention are set forth below.

In the following description, all variables are, unless otherwise noted, as defined in the formulas described herein. The following non-limiting descriptions illustrate the general methodologies that may be used to obtain the compounds described herein.

Compounds (or “prodrugs”) useful in the invention can be prepared in a variety of ways, as generally depicted in Schemes I-VI and examples of U.S. Pat. No. 6,716,825. The general phosphonate esterification methods described below are provided for illustrative purposes only and are not to be construed as limiting this invention in any manner. Indeed, several methods have been developed for direct condensation of phosphonic acids with alcohols (see, for example, R. C. Larock, Comprehensive Organic Transformations, VCH, New York, 1989, p. 966 and references cited therein). Isolation and purification of the compounds and intermediates described in the examples can be effected, if desired, by any suitable separation or purification procedure such as, for example, filtration, extraction, crystallization, flash column chromatography, thin-layer chromatography, distillation or a combination of these procedures. Specific illustrations of suitable separation and isolation procedures are in the examples below. Other equivalent separation and isolation procedures can of course, also be used.

Scheme I of U.S. Pat. No. 6,716,825 outlines a synthesis of bisphosphonate prodrugs that contain a primary amino group, such as pamidronate or alendronate. Example 1 therein provides conditions for a synthesis of 1-O-hexadecyloxypropyl-alendronate (HDP-alendronate) or 1-O-hexadecyloxypropyl-pamidronate (HDP-pamidronate). In this process, a mixture of dimethyl 4-phthalimidobutanoyl phosphonate (1b, prepared as described in U.S. Pat. No. 5,039,819)) and hexadecyloxypropyl methyl phosphite (2) in pyridine solution is treated with triethylamine to yield bisphosphonate tetraester 3b which is purified by silica gel chromatography. Intermediate 2 is obtained by transesterification of diphenyl phosphite as described in Kers, A., Kers, I., Stawinski, J., Sobkowski, M., Kraszewski, A. Synthesis, April 1995, 427 430. Thus, diphenyl phosphite in pyridine solution is first treated with hexadecyloxypropan-1-ol, then with methanol to provide compound 2.

An important aspect of the process is that other long chain alcohols may be used in place of hexadecyloxypropan-1-ol to generate the various compounds of this invention. Treatment of intermediate 3b with bromotrimethylsilane in acetonitrile cleaves the methyl esters selectively to yield monoester 4b. Treatment of 4b with hydrazine in a mixed solvent system (20% methanol/80% 1,4-dioxane) results in removal of the phthalimido protecting group as shown. The desired alendronate prodrug is collected by filtration and converted to the triammonium salt by treatment with methanolic ammonia.

Scheme II of U.S. Pat. No. 6,716,825 illustrates a synthesis of analogs of bisphosphonates lacking a primary amino group, in this case the process steps are similar to those of Scheme I except that protection with a phthalimido group and subsequent deprotection by hydrazinolysis are unnecessary. Bisphosphonates having 1-amino groups, such as amino-olpadronate, maybe converted to analogs according to the invention prodrugs using a slightly modified process shown in Scheme III of U.S. Pat. No. 6,716,825. Treatment of a mixture of compound 2 and 3-(dimethylamino)propionitrile with dry HCl followed by addition of dimethyl phosphite affords tetraester 3 which, after demethylation with bromotrimethylsilane, yields hexadecyloxypropyl-amino-olpadronate.

Scheme IV of U.S. Pat. No. 6,716,825 illustrates synthesis of a bisphosphonate analog where the lipid group is attached to a primary amino group of the parent compound rather than as a phosphonate ester.

Scheme V of U.S. Pat. No. 6,716,825 illustrates a general synthesis of alkylglycerol or alkylpropartediol analogs of cidofovir, cyclic cidofovir, and other phosphonates. Treatment of 2,3-isopropylidene glycerol, 1, with NaH in dimethylformamide followed by reaction with an alkyl methanesulfonate yields the alkyl ether, 2. Removal of the isopropylidene group by treatment with acetic acid followed by reaction with trityl chloride in pyridine yields the intermediate 3. Alkylation of intermediate 3 with an alkyl halide results in compound 4. Removal of the trityl group with 80% aqueous acetic acid affords the O,O-dialkyl glycerol, 5. Bromination of compound 5 followed by reaction with the sodium salt of cyclic cidofovir or other phosphonate-containing nucleotide yields the desired phosphonate adduct, 7. Ring-opening of the cyclic adduct is accomplished by reaction with aqueous sodium hydroxide. The compound of propanediol species may be synthesized by substituting 1-O-alkylpropane-3-ol for compound 5 in Scheme V. The tenofovir and adefovir analogs may be synthesized by substituting these nucleotide phosphonates for cCDV in reaction (f) of Scheme V. Similarly, other nucleotide phosphonates of the invention may be formed in this manner.

Scheme VI of U.S. Pat. No. 6,716,825 illustrates a general method for the synthesis of nucleotide phosphonates of the invention using 1-O-hexadecyloxypropyl-adefovir as the example. The nucleotide phosphonate (5 mmol) is suspended in dry pyridine and an alkoxyalkanol or alkylglycerol derivative (6 mmol) and 1,3-dicyclohexylcarbodiimde (DCC, 10 mmol) are added. The mixture is heated to reflux and stirred vigorously until the condensation reaction is complete as monitored by thin-layer chromatography. The mixture is then cooled and filtered. The filtrate is concentrated under reduced pressure and the residues adsorbed on silica gel and purified by flash column chromatography (elution with approx. 9:1 dichloromethane/methanol) to yield the corresponding phosphonate monoester.

Scheme VII (which is referenced as FIG. 1 in Kern et al., AAC 46 (4):991) illustrates the synthesis for alkoxyalkyl analogs of cidofovir (CDV) and cyclic cidofovir (cCDV). In FIG. 1, the arrows indicate the following reagents: (a) N,N-dicyclohexylmorpholinocarboxamide, N,N-dicyclohexylearbodiimide, pyridine, 100° C.; (b) 1-bromo-3-octadecyloxyethane (ODE), or 1-bromo-3-hexadecyloxypropane (HDP), N,N-dimethylformamide, 80° C.; (c) 0.5 M NaOH.

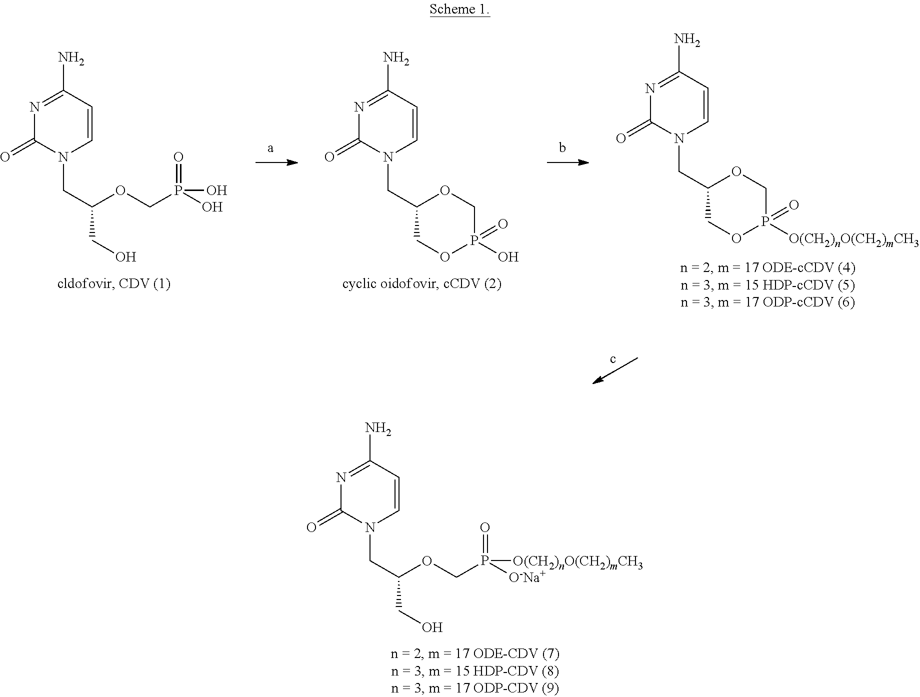

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention may be those that result in the formation of stable or chemically feasible compounds.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Active compounds as described herein may also be prepared in accordance with known procedures, or variations thereof that will be apparent to those skilled in the art. See, e.g., Painter et al., Evaluation of Hexadecyloxypropyl-9-R-[2-(Phosphonomethoxy)Propyl]-Adenine, CMX157, as a Potential Treatment for Human Immunodeficiency Virus Type I and Hepatitis B Virus Infections, Antimicrobial Agents and Chemotherapy 51, 3505-3509 (2007) and US Patent Application Publication No. 2007/0003516 to Almond et al.

CMX157 may be prepared in accordance with known procedures, or variations thereof that will be apparent to those skilled in the art. See, e.g., Painter et al., Evaluation of Hexadecyloxypropyl-9-R-[2-(Phosphonomethoxy)Propyl]-Adenine, CMX157, as a Potential Treatment for Human Immunodeficiency Virus Type 1 and Hepatitis B Virus Infections, Antimicrobial Agents and Chemotherapy 51, 3505-3509 (2007) and US Patent Application Publication No. 2007/0003516 to Almond et al.

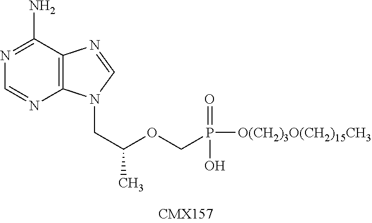

In one embodiment, the compound described herein may be prepared by dissolving compound 1 in an appropriate solvent,

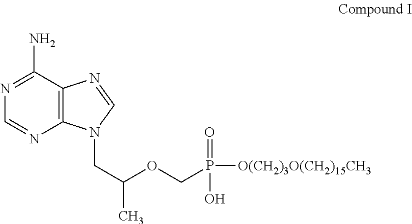

adding a suitable base to the mixture of the solvent and compound 1, and removing the solvent to provide a salt of formula I.

The solvent used in the preparation may be any suitable solvent known to one skilled in the art or a combination of solvents that provides satisfactory yield of the product. In one embodiment, the solvent is a mixture of at least two solvents. Exemplary combination of solvents includes, but is not limited to, dichloromethane and methanol, dichloromethane and ethanol. In one embodiment, the molar ratio of the dichloromethane and methanol is in a range of about 1:1 to 9:1. In one embodiment, the molar ratio of the dichloromethane and methanol is in a range of about 7:3 to 9:1. In a further embodiment, the molar ratio of the dichloromethane and methanol is about 9:1.

The base used in the preparation may be any suitable base known to one skilled in the art or a combination of bases that provides satisfactory yield of the product. In some embodiments, the base is an alkali metal alcoholate base. Exemplary bases include, but are not limited to, potassium methoxide, sodium methoxide, lithium ter-butoxide, ammonium hydroxide, sodium hydroxide, potassium hydroxide, and lithium hydroxide.

The process described herein may further include the step of recrystallization to remove impurity, side products, and unreacted starting material. The recrystallization step comprises the step of dissolving the product in a suitable solvent at an appropriate temperature, cooling to an appropriate temperature for a sufficient period of time to precipitate the compound of formula I, filtering to provide the compounds of formula I. In some embodiments, the temperature for the step of dissolving is in a range of about 50° C. to 80° C.

### C. PHARMACEUTICAL FORMULATIONS AND ADMINISTRATION

In one embodiment, the present invention is a pharmaceutical composition comprising the compounds described herein. In another embodiment, the pharmaceutical composition further comprises a pharmaceutically acceptable carrier. The term “pharmaceutically acceptable carrier” as used herein refers to any substance, not itself a therapeutic agent, used as a vehicle for delivery of a therapeutic agent to a subject. Examples of pharmaceutically acceptable carriers and methods of manufacture for various compositions include, but are not limited to, those described in Remington's Pharmaceutical Sciences, 18th Ed., Mack Publishing Co. (1990) (See also US Patent Application US 2007/0072831).

In some embodiments, the pharmaceutical composition further comprises one or more immunosuppressive agents described in Section E.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Any suitable route of administration may be employed for providing a mammal, especially a human with an effective dosage of a compound of the present invention. For example, the compositions of the present invention may be suitable for formulation for oral, parenteral (including subcutaneous, intramuscular, intravenous, intradermal, intrathecal and epidural), inhalation spray, topical, rectal, nasal, sublingual, buccal, vaginal or implanted reservoir administration, etc. in some embodiments, the compositions are administered orally, topically, intraperitoneally or intravenously. These suspensions may be formulated according to techniques known in the art using suitable dispersing or wetting agents and suspending agents. Among the acceptable vehicles and solvents that may be employed are water, Ringer's solution and isotonic sodium chloride solution. In addition, sterile, fixed oils are conventionally employed as a solvent or suspending medium.

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

A pharmaceutically acceptable oil may be employed as a solvent or suspending medium in compositions of the present invention. Fatty acids, such as oleic acid and its glyceride derivatives are suitably included in injectable formulations, as are natural pharmaceutically acceptable oils, such as olive oil or castor oil, especially in their polyoxyethylated versions. The oil containing compositions of the present invention may also contain a long-chain alcohol diluent or dispersant, such as carboxymethyl cellulose or similar dispersing agents that are commonly used in the formulation of pharmaceutically acceptable dosage forms including emulsions and suspensions. The compositions suitably further comprise surfactants (such as non-ionic detergents including Tween® or Span®) other emulsifying agents, or bioavailability enhancers.

The compositions of this invention may be in the form of an orally acceptable dosage form including, but not limited to, capsules, tablets, suspensions or solutions. The oral dosage form may include at least one excipient. Excipients used in oral formulations of the present can include diluents, substances added to mask or counteract a disagreeable taste or odor, flavors, dyes, fragrances, and substances added to improve the appearance of the composition. Some oral dosage forms of the present invention suitably include excipients, such as disintegrants, binding agents, adhesives, wetting agents, polymers, lubricants, or glidants that permit or facilitate formation of a dose unit of the composition into a discrete article such as a capsule or tablet suitable for oral administration. Excipient-containing tablet compositions of the invention can be prepared by any suitable method of pharmacy which includes the step of bringing into association one or more excipients with a compound of the present invention in a combination of dissolved, suspended, nanoparticulate, microparticulate or controlled-release, slow-release, programmed-release, timed-release, pulse-release, sustained-release or extended-release forms thereof.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc.), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, rings, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Pharmaceutically acceptable compositions of the present invention may be in the form of a topical solution, ointment, or cream in which the active component is suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Where the topical formulation is in the form of an ointment or cream, suitable carriers include, but are not limited to, mineral oil, sorbitan monostearate, polysorbate 60, cetyl esters wax, cetearyl alcohol, 2 octyldodecanol, benzyl alcohol and water. In some embodiments, the topical composition of the present invention is in the form of a spray.

The pharmaceutically acceptable compositions of this invention may also be administered by nasal, aerosol or by inhalation administration routes. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments, the nasal administration of the composition of the present invention is in the form of a spray. Any suitable carrier for spray application may be used in the present invention.

Alternatively, pharmaceutically acceptable compositions of this invention may be in the form of a suppository for rectal administration. The suppositories can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Additionally, the pharmaceutical formulation including compounds of the present invention can be in the form of a parenteral formulation. The term “parenteral” as used herein includes subcutaneous, intravenous, intramuscular, intra-articular, intra-synovial, intrasternal, intrathecal, intrahepatic, intralesional and intracranial injection or infusion techniques.

In certain embodiments, the pharmaceutically compositions of this invention are formulated for oral administration. For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions may be provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

It should also be understood that a specific dosage and treatment regimen for any particular patient will depend upon a variety of factors, including the activity of the specific compound employed, the mode of administration, the age, body weight, general health, gender, diet, rate of excretion, drug combination, and the judgment of the treating physician, the condition being treated and the severity of the condition. Such dosage may be ascertained readily by a person skilled in the art. This dosage regimen may be adjusted to provide the optimal therapeutic response.

The present invention further provides veterinary compositions comprising at least one active ingredient as above defined together with a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can be used to provide controlled release pharmaceutical formulations containing as active ingredient one or more compounds of the invention (“controlled release formulations”) in which the release of the active ingredient can be controlled and regulated to allow less frequent dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for treating various viral infections and/or diseases associated with virus. Exemplary diseases associated with virus include, but are not limited to, diseases associated with at least one virus selected from polyomavirus (including BK, John Cunningham virus (JCV), Merkel cell virus (MCV), KI polyomavirus (KIV), WU polyomavirus (WUV), Simian virus 40 (SV 40)), papillomavirus (including human papillomavirus, cottontail rabbit papillomavirus, equine papillomavirus and bovine papillomavirus), herpes virus, adenovirus, Epstein-Barr virus (EBV), human cytogegalovirus (HCMV), Hepatitis B virus, Hepatitis C virus, varicella zoster virus (VZV) or a combination thereof. The controlled release formulations can also be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotropic virus and HIV-2 infections. The invention accordingly provides pharmaceutical formulations for treating the above-mentioned human or veterinary conditions.

Pharmacokinetic enhancers. The compounds of the invention may be employed in combination with pharmacokinetic enhancers (sometimes also referred to as “booster agents”). One aspect of the invention provides the use of an effective amount of an enhancer to enhance or “boost” the pharmacokinetics of a compound of the invention. An effective amount of an enhancer, for example, the amount required to enhance an active compound or additional active compound of the invention, is the amount necessary to improve the pharmacokinetic profile or activity of the compound when compared to its profile when used alone. The compound possesses a better efficacious pharmacokinetic profile than it would without the addition of the enhancer. The amount of pharmacokinetic enhancer used to enhance the potency of the compound is, preferably, subtherapeutic (e.g., dosages below the amount of booster agent conventionally used for therapeutically treating infection in a patient). An enhancing dose for the compounds of the invention is subtherapeutic for treating infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase, RT or protease and/or reduced systematic clearance. One example of a pharmacokinetic enhancer is RITONAVIR™ (Abbott Laboratories).

Combinations. As noted above, the compositions of the present invention can include the active compounds as described in section A above in combination with one or more (e.g., 1, 2, 3) immunosuppressant agents such as described in section E below, in analogous manner as known in the art.

Specific examples of such combinations include, but are not limited to: CMX001 or a pharmaceutically acceptable salt thereof in combination with at least one immunosuppressant agents. Exemplary immunosurpressant agent include, but are not limited to, Daclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm and a combination thereof. In some embodiments, the pharmaceutical composition includes CMX001, Tysabri (natalizumab), and a pharmaceutically acceptable carrier.

In one embodiment, the pharmaceutical composition described herein comprises CMX001, or pharmaceutically acceptable salt thereof and one or more medication that cause PML in at least one pharmaceutically acceptable carrier. In one embodiment, one or more medication is selected from the group consisting of Rituxan, Raptiva, Tysabri (natalizumab), Myfortic, Avonex, Remicade, Enbrel, Humira, Cellcept and a combination thereof in at least one pharmaceutically acceptable carrier.

In another embodiment, the pharmaceutical composition described herein includes CMX001 and CMX 157 or a pharmaceutically acceptable salt of any thereof, in at least one pharmaceuticaly acceptable carrier.

### D. METHODS OF USE

One aspect of the present invention provides methods of treating conditions/disease associated with at least one virus in a subject which includes administering to the subject a therapeutically effective amount of a compound described herein.

In one embodiment, the compounds described herein specifically target against viral replication and/or virally infected/transformed cells. For example, CMX001 demonstrates specificity against polyomavirus infected cells such as BK virus and JC virus infected cells. In one embodiment, the compounds described herein have a higher cytotoxicity against virally infected and/or transformed cells compared to normal (uninfected cells).

In some embodiments, the disease associated with virus is selected from nephropathy, hemorrhagic cystitis, or progressive multifocal leukoencephalopathy (PML). In another embodiment, nephropathy, hemorrhagic cystitis is associated with at least one polyomavirus (e.g., BK virus or JC virus). Further, in one embodiment, hemorrhagic cystitis is associated with at least one adenovirus (e.g., serotypes 11 and 12 of subgroup B). In one embodiment, the progressive multifocal leukoencephalopathy (PML) is associated with at least JC virus.

In some embodiments, the disease is associated with at least one virus selected from polyomavirus (including BK, John Cunningham virus (JCV), Merkel cell virus (MCV), KI polyomavirus (KW), WU polyomavirus (WUV), Simian virus 40 (SV 40)), papillomavirus (including human papillomavirus, cottontail rabbit papillomavirus, equine papillomavirus and bovine papillomavirus), herpes virus, adenovirus, Epstein-Barr virus (EBV), human cytomegalovirus (HCMV), Hepatitis B virus, Hepatitis C virus or a combination thereof.

In another embodiment, the disease is associated with at least one virus selected from the group consisting of human immunodeficiency virus (HIV), influenza, herpes simplex virus 1, herpes simplex virus 2, human herpes virus 6 (HHV-6), human herpes virus 8 (HHV-8)), cytomegalovirus (CMV), hepatitis B and C virus, Epstein-Barr virus (EBV), varicella zoster virus, variola major and minor, vaccinia, smallpox, cowpox, camelpox, monkeypox, ebola virus, papilloma virus, adenovirus or polyoma virus including JC virus, BK virus, SV40, and a combination thereof.

In some embodiments, the disease is associated with at least one virus that is BK virus or JCV.

In one embodiment, the subject is human. In one embodiment, the subject is an immunocompromised subject. In one embodiment, the subject is in need of a chemtherapy agent.

In some embodiments, the subject has been previously treated with at least one antiviral agents and the previous treatment has failed and the previously used antiviral agent is selected from the group consisting of cidofovir, ganciclovir, valganciclovir, foscarnet, acyclovir, valacyclovir and a combination thereof. In another embodiment, the present invention provides methods of treating conditions/disease associated with at least one virus in a subject, wherein treatment with cidofovir alone has failed. In another embodiment, the present invention provides methods of treating conditions/disease associated with at least one virus in a subject where treatment with ganciclovir alone has failed. In another embodiment, the present invention provides methods of treating conditions/disease associated with at least one virus in a subject where treatment with cidofovir and/or ganciclovir alone or in combination has failed.

In another embodiment, the disease is associated with herpes virus and the subject has been previously treated with at least one antiviral agents and the previous treatment has failed and the previously used antiviral agent is selected from the group consisting of cidofovir, ganciclovir, valgancielovir, foscarnet, acyclovir, valacyclovir and a combination thereof. In another embodiment, the present invention provides methods of treating a herpes virus infection where treatment with acyclovir alone has failed. In another embodiment, the present invention provides methods of treating a herpes virus infection where treatment with valacyclovir alone has failed. In another embodiment, the present invention provides methods for treating a herpes virus infection where treatment with acyclovir and/or valacyclovir alone or in combination has failed.

In another embodiment, the disease is associated with adenovirus virus and the subject has been previously treated with at least one antiviral agents and the previous treatment has failed and the previously used antiviral agent is selected from the group consisting of cidofovir, ganciclovir, valganciclovir, foscarnet, acyclovir, valacyclovir and a combination thereof.

In another embodiment, the disease is associated with at least with one herpes virus and the methods comprise administering a compound (CMX001) having the structure

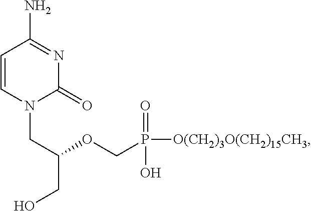

or a pharmaceutically acceptable salt thereof, and/or in combination with at least one antiviral agent selected from the group consisting of ganciclovir, valganciclovir, foscarnet, acyclovir, valacyclovir, and a combination thereof. In another embodiment, the present invention provides methods of treating a herpes virus infection with a combination of CMX001 and acyclovir.

Further, in one embodiment, the disease is associated with at least one cytomegalovirus and the methods comprise administering a compound (CMX001) having the structure

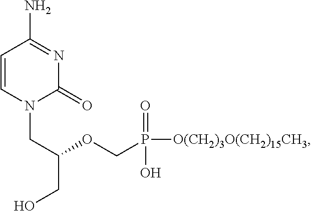

or a pharmaceutically acceptable salt thereof, and/or in combination with at least one antiviral agent selected from the group consisting of ganciclovir, valganciclovir, foscarnet, acyclovir, valacyclovir and a combination thereof. In another embodiment, the present invention provides methods of treating a cytomegalovirus infection with a combination of CMX001 and ganciclovir.

In one embodiment, the disease is associated with at least one adenovirus and the methods comprise administering a compound (CMX001) having the structure

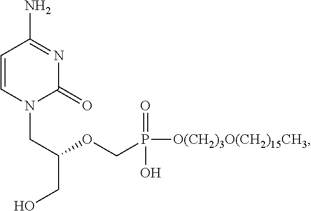

or a pharmaceutically acceptable salt thereat and/or in combination with at least one antiviral agent selected from the group consisting of ganciclovir, valganciclovir, foscarnet, acyclovir, valacyclovir and a combination thereof.

In another embodiment, the present invention provides methods of treating an adenovirus infection with CMX001. In another embodiment, the present invention provides methods of treating an adenovirus infection in vivo. In another embodiment, the present invention provides methods of treating an adenovirus infection in vivo with CMX001.

As used herein, immunodeficiency (or immune deficiency) is a state in which the immune system's ability to fight infectious disease is compromised or entirely absent. An immunocompromised subject is a subject that has an immunodeficiency of any kind or of any level. An immunocompromised person may be particularly vulnerable to opportunistic infections, in addition to normal infections. Exemplary immunocompromised subject includes, but are not limited to, a subject with primary immunodeficiency (a subject that is born with defects in immune system) and a subject with secondary (acquired) immunodeficiency. In addition, other common causes for secondary immunodeficiency include, but are not limited to, malnutrition, aging and particular medications (e.g. immunosuppressive therapy, such as chemotherapy, disease-modifying antirheumatic drugs, immunosuppressive drugs after organ transplants, glucocorticoids). Other exemplary diseases that directly or indirectly impair the immune system include, but are not limited to, various types of cancer, (e.g. bone marrow and blood cells (leukemia, lymphoma, multiple myeloma)), acquired immunodeficiency syndrome (AIDS) caused by human immunodeficiency virus (HIV), chronic infections and autoimmune diseases (e.g. Acute disseminated encephalomyelitis (ADEM), Addison's disease, Alopecia areata, Ankylosing spondylitis, Antiphospholipid antibody syndrome (APS), Autoimmune hemolytic anemia, Autoimmune hepatitis, Autoimmune inner ear disease, Bullous pemphigoid, Coeliac disease, Chagas disease, Chronic obstructive pulmonary disease, Crohns Disease, Dermatomyositis, Diabetes mellitus type 1, Endometriosis, Goodpasture's syndrome, Graves' disease, Guillain-Barré syndrome (GBS), Hashimoto's disease, Hidradenitis suppurativa, Kawasaki disease, IgA nephropathy, Idiopathic thrombocytopenic purpura, Interstitial cystitis, Lupus erythematosus, Mixed Connective Tissue Disease, Morphea, Multiple sclerosis (MS), Myasthenia gravis, Narcolepsy, Neuromyotonia, Pemphigus vulgaris, Pernicious anaemia, Psoriasis, Psoriatic Arthritis, Polymyositis, Primary biliary cirrhosis, Rheumatoid arthritis, Schizophrenia, Scleroderma, Sjögren's syndrome, Stiff person syndrome, Temporal arteritis (also known as “giant cell arteritis”), Ulcerative Colitis, Vasculitis, Vitiligo, Wegener's granulomatosis.)

In some embodiments, the compound described herein is administered to said subject at a dosage of less than 1 mg/Kg; in some embodiments the conjugate compound is administered to said subject at a dosage of 0.01, 0.05, 0.1, 0.2, 0.3, or 0.5 to 5, 10, 15 or 20 mg /Kg.

In some embodiments, the compounds described herein may be useful in treating subjects afflicted with at least two different dsDNA which synergistically activate one another (e.g., CMV and HIV virus in combination, CMV and BK virus in combination; etc.) See, e.g., L T Feldman et al., PNAS, Aug. 15, 1982, 4952-4956; B. Bielora et al., Bone Marrow Transplant, 2001 September; 28(6): 613-4.

In one embodiment, the subject is a transplant patient (including, but is not limited to, a renal transplant patient, a bone marrow transplant patient, a hepatic transplant patient, a liver transplant patient, a stem cell transplant patient, a lung transplant patient, a pancreas transplant patient, and/or a heart transplant patient) on immunosuppressive agent.

In some embodiments, the present invention is applied to a subject on immunosuppressive medications, (e.g. transplant patient or subjects that are suffering from an over-active immune system), a subject receiving certain kinds of chemotherapy, or a subject that is infected with human immunodeficiency virus (HIV). In one embodiment, the present invention is applied to a subject on at least one chemtherapy medication.

Another aspect of the invention provides methods of treating progressive multifocal leukoencephalopathy (PML) comprising administering to a subject a therapeutically effective amount of compound having the structure of

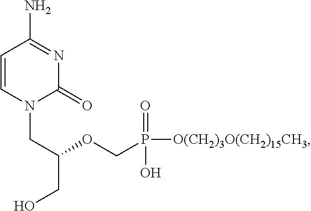

or a pharmaceutically acceptable salt thereof, wherein the subject is administered in combination with at least another medication that causes PML. In one embodiment, the medication is selected from the group consisting of Rituxan, Raptiva, Tysabri (natalizumab), Myfortic, Avonex, Remicade, Enbrel, Humira, and Cellcept.

According to one aspect of the present invention, it provides a method of treating multiple sclerosis and/or progressive multifocal leukoencephalopathy (PML). The method comprises administering to a subject a therapeutically effective amount of compound having the structure of

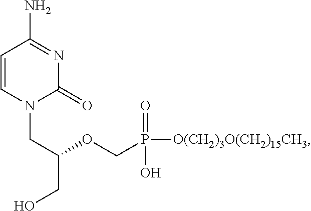

or a pharmaceutically acceptable salt thereof and in combination with Tysabri (natalizumab).

According to another embodiment of the invention, it provides methods of treating HIV and/or disorders associated with at least one virus in a subject comprising administering to the subject a therapeutically effective amount of a compound of

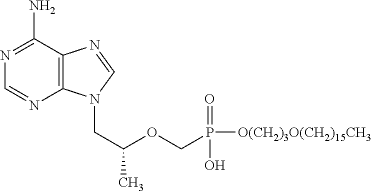

or a pharmaceutically acceptable salt of any thereof, and in combination with

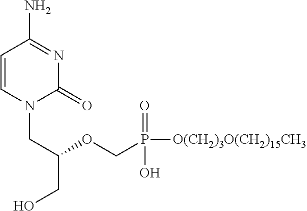

or a pharmaceutically acceptable salt of any thereof.

In some embodiments, the disease associated with at least one virus is selected from the group consisting of nephropathy, hemorrhagic cystitis, and progressive multifocal leukoencephalopathy (PML). In another embodiment, the disease is associated with at least one virus that is polyomavirus or adenovirus. In one embodiment, the disease is associated with at least one virus selected from the group consisting of BK, John Cunningham virus (JCV), Merkel cell virus (MCV), KI polyomavirus (KIV), WU polyomavirus (WUV), Simian virus 40 (SV 40) and a combination thereof. In one embodiment, the disease is associated with at least one virus that is BK virus or JCV.

### E. COMBINATION WITH IMMUNOSUPPRESSANT AGENTS FOR TREATING DISEASES ASSOCIATED WITH VIRUS

The compounds described herein may be used in combination (concurrently or sequentially) with additional immunosuppressive agents to treat diseases associated with a virus of a subject that is in need of immunosuppressant medications. Any appropriate immunosuppressive agent may be used in combination with compounds described herein. As used herein, immunosuppressive medications are described in Section E above and used in an amount effective to provide an immunosuppressant effect.

Any immunosuppressive agents known to one skilled in the art may be used in combination with the compounds described herein. Exemplary immunosuppressive agents include, but are not limited to, aclizumab, Basiliximab, Tacrolimus, Sirolimus, Mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept, Adalimumab, Tysabri (Natalizumab), Fingolimodm or a combination thereof. In one embodiment, CMX001, or a pharmaceutically acceptable salt thereof may be administered in combination with at least one immunosuppressant agents that is Tysabri (natalizumab) to treat diseases associated with virus.

Additional exemplary immunosuppressant agents are further described in Mukherjee et al., A comprehensive review of immunosuppression used for liver transplantation, Journal of Transplantation, vol. 2009, article ID 701464 and Woodroffe et al., Clinical and cost-effectiveness of newer immunosuppressive regimens in renal transplantation: a systematic review and modeling study, Health Technology Assessment, vol. 9, No. 21(2005).

### F. EXAMPLES

The present invention is explained in greater detail in the following non-limiting Examples.

The present invention will now be described in more detail with reference to the following examples. However, these examples are given for the purpose of illustration and are not to be construed as limiting the scope of the invention.

In the following Examples, CMX001 is

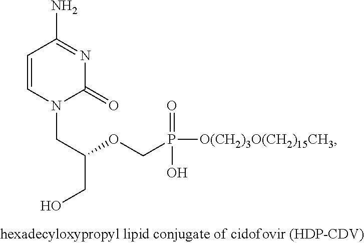

or a pharmaceutically acceptable salt thereof.

In the following Examples, cidofovir (also referred as “CMX021”) is

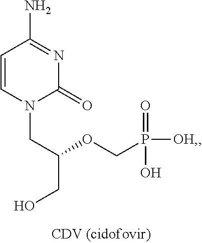 [SMILES: Nc1ccn(C[C@@H](CO)OCP(=O)(O)O)c(=O)n1]

or a pharmaceutically acceptable salt thereof.

In the following Examples, CMX064 has the structure:

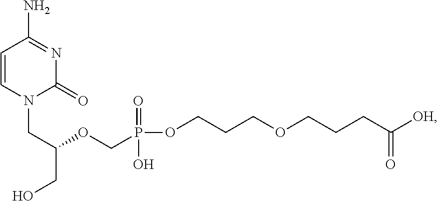 [SMILES: Nc1ccn(C[C@@H](CO)OCP(=O)(O)OCCCOCCCC(=O)O)c(=O)n1]

or a pharmaceutically acceptable salt thereof.

### Examples of Preparation of the Compounds Described Herein

### (1) Synthesis of the Hexadecyloxypropyl, Octadecyloxypropyl, Octadecyloxyethyl and Hexadecyl Esters of Cyclic Cidofovir

To a stirred suspension of cidofovir (1.0 g, 3.17 mmol) in N,N-DMF (25 mL) was added N,N-dicyclohexyl-4-morpholine carboxamidine (DCMC, 1.0 g, 3.5 mmol). The mixture was stirred overnight to dissolve the cidofovir. This clear solution was then charged to an addition funnel and slowly added (30 min.) to a stirred, hot pyridine solution (25 mL, 60° C.) of 1,3-dicyclohexyl carbodiimide (1.64 g, 7.9 mmol). This reaction mixture was stirred at 100. degree. C. for 16 h then cooled to room temperature, and the solvent was removed under reduced pressure. The residue was adsorbed on silica gel and purified by flash column chromatography using gradient elution (CH₂Cl₂+MeOH). The UV active product was finally eluted with 5:5:1 CH₂Cl₂ /MeOH/H₂O Evaporation of the solvent gave 860 mg of a white solid. The ¹H and ³¹P NMR spectrum showed this to be the DCMC salt of cyclic cidofovir (yield=44%).

To a solution of cyclic cidofovir (DCMC salt) (0.5 g, 0.8 mmol) in dry DMF (35 mL) was added 1-bromo-3-hexadecyloxypropane (1.45 g, 4 mmol) and the mixture was stirred and heated at 80° C. for 6 h. The solution was then concentrated in vacuo and the residue adsorbed on silica gel and purified by flash column chromatography using gradient elution (CH₂Cl₂+EtOH). The alkylated product was eluted with 90:10 CH₂Cl₂/EtOH. The fractions containing pure product were evaporated to yield 260 mg HDP-cyclic cidofovir (55% yield).

To a solution of cyclic cidofovir (DCMC salt) (1.0 g, 3.7 mmol) in dry DMF (35 mL) was added 1-bromo-3-octadecyloxypropane (2.82 g, 7.2 mmol) and the mixture was stirred and heated at 85° C. for 5 h. The solution was then concentrated in vacuo and the residue adsorbed on silica gel and purified by flash column chromatography using gradient elution (CH₂Cl₂+MeOH). The alkylated product was eluted with 9:1 CH₂Cl₂/MeOH. The fractions containing pure product were evaporated to yield 450 mg ODP-cyclic cidofovir.

To a solution of cCDV (DCMC salt) (1.0 g, 3.7 mmol) in dry DMF (35 mL) was added 1-bromo-3-octadecyloxyethane (3.0 g, 7.9 mmol) and the mixture was stirred and heated at 80° C. for 4 h. The solution was then concentrated in vacuo and the residue adsorbed on silica gel and purified by flash column chromatography using gradient elution (CH₂Cl₂+MeOH). The alkylated product was eluted with 9:1 CH₂Cl₂/MeOH. The fractions containing pure product were evaporated to yield 320 mg octadecyloxyethyl-cCDV.

To a solution of cyclic cidofovir (DCMC salt) (0.5 g, 0.8 mmol) in dry DMF (35 mL) was added 1-bromo-hexadecane (1.2 g, 4 mmol) and the mixture was stirred and heated at 80° C. for 6 h. The solution was then concentrated in vacuo and the residue adsorbed on silica gel and purified by flash column chromatography using gradient elution (CH₂Cl₂+MeOH). The alkylated product was eluted with 9:1 CH₂Cl₂/MeOH. The fractions containing pure product were evaporated to yield 160 mg hexadecyl-cCDV.

### (2) Synthesis of the Hexadecyloxypropyl, Octadecyloxypropyl, Octadecyloxyethyl and Hexadecyl Esters of Cidofovir

Hexadecyloxypropyl-cyclic CDV from above was dissolved in 0.5M NaOH and stirred at room temp for 1.5 h. 50% aqueous acetic was then added dropwise to adjust the pH to about 9. The precipitated HDP-CDV was isolated by filtration, rinsed with water and dried, then recrystallized (3:1 p-dioxane/water) to give HDP-CDV.

Similarly, the octadecyloxypropyl-, octadecyloxyethyl- and hexadecyl-cCDV esters were hydrolyzed using 0.5M NaOH and purified to give the corresponding cidofovir diesters.

### (3) Preparation of the Salts of CMX157

The free acid form of CMX157 may be prepared by methods known to one skilled in the art (See e.g., Painter et al., Evaluation of hexadecyloxypropyl-9-R-[2-(Phosphonomethoxy)propyl]-adenine, CMX157, as a potential treatment for human immunodeficiency virus type 1 and hepatitis B virus infections. Antimicrob Agents Chemother 51:3505-9 (2007), and Painter, et al., Design and development of oral drugs for the prophylaxis and treatment of smallpox infection. Trends Biotechnol 22:423-7 (2004).)

CMX157 Sodium Salt The free acid form of CMX157 (55.0 grams, 96.5 mmol) is dissolved in solution of DCM:MeOH (9:1, 550 mL) at room temperature. Sodium methoxide (0.5M solution in methanol, 193.1 mL, 96.5 mmol) is added to the solution and stirred at room temperature for 30 minutes. The reaction mixture is concentrated in vacuo to dryness (50° C. water bath). The resulting off-white foam is dissolved in ethanol (200 mL) at 60° C., diluted with acetone (200 mL), cooled to room temperature, and aged for 18 hours. The suspension is held at 5° C. for 48 hours, filtered, washed with acetone (200 mL), and dried in vacuo at 35° C. for 48 hours to yield CMX157-sodium salt 54.4 g (95.2%) as a white solid. HPLC (AUC) purity 99.6%.

CMX157 Potassium Salt The free acid form of CMX157 (55.0 grams, 96.5 mmol) is dissolved in solution of DCM:MeOH (9:1, 550 mL) at room temperature. Potassium methoxide (25% solution in methanol, 28.5 mL, 96.5 mmol) is added to the solution and stirred at room temperature for 30 minutes. The reaction mixture is concentrated in vacuo to dryness (50° C. water bath). The resulting off-white foam is dissolved in ethanol (200 mL) at 60° C., diluted with acetone (200 mL), cooled to room temperature, and aged for 18 hours. The suspension is held at 5° C. for 48 hours, filtered, washed with acetone (200 mL), and dried in vacuo at 35° C. for 48 hours to yield CMX157-potassium salt 48.4 g (82.4%) as a white solid. HPLC (AUC) purity 97.4%.

CMX157 Lithium Salt The free acid form of CMX157 (55.0 grams, 96.5 mmol) is dissolved in solution of DCM:MeOH (9:1, 550 mL) at room temperature. Lithium tert-butoxide (7.73 g, 96.5 mmol) is added to the solution and stirred at room temperature for 30 minutes. The reaction mixture is concentrated in vacuo to dryness (50° C. water bath). The resulting off-white solid is dissolved in ethanol (800 mL) at 70° C., cooled to room temperature, and aged for 16 hours. The fine suspension is filtered, washed with acetone (200 mL), and dried in vacuo at 35° C. for 48 hours to yield CMX157-lithium salt 51.2 g (92.1%) as a white solid. HPLC (AUC) purity 95.7%.

CMX157 Ammonium Salt The free acid form of CMX157 (55.0 grams, 96.5 mmol) is dissolved in 2-propanol (220 mL) at 78° C. in the presence of ammonium hydroxide (28-30% solution 13.54 mL, 96.5 mmol). The reaction mixture is cooled to room temperature, and aged for 18 hours. The suspension is held at 5° C. for 48 hours, filtered, and air dried for approximately 48 hours to yield CMX157-ammonium salt 51.7 g (91.3%) as a white solid. HPLC (AUC) purity 98.7%.

### EXAMPLE II

### Preclinical Studies of CMX001

### Example 1

As summarized in Tables 1-2 below, pre-clinical studies of CIVLX001 indicate that it is essentially completely protective against lethal Orthopoxvirus infections in mice and rabbits. The effective dose in these animal models ranges from 1-2 mg/kg daily for 5 days in low titer inoculums, while late stage requires 20-30 mg/kg as a single dose.

TABLE 1
CMX001 has Enhanced In Vitro Potency Against dsDNA Viruses.

| | Cell | Cidofovir | CMX001 | Enhanced |
|---|---|---|---|---|
| Virus | Line | EC50 (μm) | EC50 (μM) | Activity |
| Variola major | Vero 76 | 27.3 | 0.1 | 271 |
| Vaccinia Virus | HFF | 46 | 0.8 | 57 |
| HCMV(AD169) | MRC-5 | 0.38 | 0.0009 | 422 |
| BK Virus | WI-38 | 115.1 | 0.13 | 885 |
| HSV-1 | MRC-5 | 15 | 0.06 | 250 |
| HHV-6 | HSB-2 | 0.2 | 0.004 | 50 |
| Adenovirus | HFF | 1.3 | 0.02 | 65 |
| HPV 18 | HeLa | 516 | 0.42 | 1229 |
| HPV 11 | A431 | 716 | 17 | 42 |
| EBV | Dardi | >170 | 0.04 | >4250 |

TABLE 2
CMX001 is protective against lethal orthopoxvirus
infections in mice and rabbits.

| | Viral Inoculum | 100% Protective |
|---|---|---|
| | (PFU) | Dose of CMX001* |
| Mice Infected | 1.2 | 1 mg/kg/day |
| with Ectromelia | 27 | 4 mg/kg/day |
| | 270 | 4 mg/kg/day |
| | 9200 | 8 mg/kg/day |
| Rabbits Infected | 100 | 2 mg/kg/day |
| with Rabbitpox | 500 | 10 mg/kg/day |
| | 1000 | 20 mg/kg/day |

*Dose was orally administered for five consecutive days

In addition, over twenty-one toxicology studies have been conducted in mice, rats, rabbits and monkeys with CMX001 being delivered by the oral route. In none of these studies (as opposed to the delivery of efficacious doses of cidofovir by i.v.), has there been any indication of nephrotoxicity (see, e.g., Example 2 below).

### Example 2

To test the ability of CMX001 to inhibit replication of BK virus, stocks of BK virus were prepared in HFF cells and dilutions of the virus stocks were used to infect primary human renal tubular epithelial cells (RPTECs). Drug dilutions were then added to the wells containing the infected cells and the plates were incubated for 5 days. Total DNA was prepared from the plates and viral DNA was quantified by qPCR. CMX001 exhibited good activity against BK virus in RPTECs and was more potent than cidofovir (Table 3(a)). The negative control drug, ganciclovir, was essentially inactive. The assay optimization in the cell line also revealed that the multiplicity of infection appeared to impact the efficacy of cidofovir and CMX001.

TABLE 3(a)
Antiviral activity of CMX001 against BK virus in RPTEC cells

| Virus Dilution | cidofovir EC₅₀ | CMX001 EC₅₀ | ganciclovir EC₅₀ |
|---|---|---|---|
| 1:10 | 2.0 | 0.016 | 89.0 |
| 1:50 | 0.65 | 0.0035 | >100 |
| 1:100 | 0.44 | 0.0037 | 70.8 |

All EC₅₀ values are in μM.

To test the ability of CMX001 to inhibit JC virus, COS-7 cells were infected with JCV (Mad-4) at an estimated TCID50 of 0.2. After a 2 h incubation at 37° C., supernatants were replaced with fresh medium without or with increasing amounts of CMX001 and incubated for 5 days. JC virus was quantified by qPCR after DNA extraction. As shown in Table 3(b), CMX001 was active against JC virus.

TABLE 3(b)
Antiviral activity of CMX001 against JC
virus in COS-7 cells.

| CMX001 EC₅₀ | CMX001 EC₉₀ |
|---|---|
| 0.15 | 0.6 |

All values are in μM.

### Example 3

RPTECs were infected with BKV(Dunlop). CMX001 was added before and 2 h postinfection (hpi). Cells and supernatants were harvested 24-72 hpi. BKV replication was examined by TaqMan assays, western blotting, IF staining and viability of RPTECs was examined by WST-1 assay, BrdU incorporation and a TaqMan assay.

CMX001 0.31 μM reduced extracellular BKV loads by 90% at 72 hpi. At this concentration we observed a 30% reduction in BrdU incorporation while WST-1 activity was unchanged. BKV entry and early expression was unaffected but BKV DNA replication was reduced by 94% at 48 hpi, Late protein expression was about 70% reduced.

CMX001 inhibits BKV replication at the level of DNA replication. CMX001 031 uM gives a 90% reduction of extracellular BKV loads. CMX001 has a longer lasting effect than CDV at 400× lower levels with less effects on metabolic activity and cellular DNA replication less.

### Example 4

### CMX001 Inhibits Polyomavirus BK Replication in Primary Human Renal Tubular Cells

A. Materials and Methods

Primary human renal proximal tubule epithelial cells (RPTECs), BKV(Dunlop) and all methods as previously described by Bernhoff et al (See Bernhoff et al., Cidofovir inhibits polyomavirus BK replication in human renal tubular cells downstream of viral early gene expression, Am J Transplant 8, 1413-1422 (2008).) Only one exception, quantitative PCR (qPCR) to quantify intracellular or extracellular BKV DNA load was performed with a different primer/probe set also targeting the LTag gene (See Hirsch, et al., J. Prospective study of polyomavirus type BK replication and nephropathy in renal-transplant recipients, N Engl J Med., 347, 488-496 (2002)). Before each experiment, CMX001 was freshly dissolved to 1 mg/ml in methanol/water/ammonium hydroxide (50/50/2). It was further diluted in RPTEC growth medium.

B. Experiments and Results

(1) Determination of Inhibitory Concentration IC₉₀

To investigate the effect of CMX001 on BKV progeny, increasing concentrations of CMX001 were added 2 h p.i. and supernatants harvested at 72 h p.i. It was observed that CMX001 reduced the extracellular BKV load in a concentration dependent manner (See FIG. 1a). When viral input was subtracted, CMX001 0.31 μM reduced the BKV load by an average of 90% defining the inhibitory concentration IC₉₀. Immunofluorescence staining 72 h p.i. of BKV-infected RPTECs demonstrated decreasing numbers of BKV-infected cells with increasing CMX001 concentration (FIG. 1b). With CMX001 0.31 uM an approximately 60% decrease in BKV agnoprotein expressing cells was seen. The number of cells expressing LTag was less reduced but the signal intensity was lower than in untreated cells. With 2.5 uM CMX001 only few cells were positive for agnoprotein and LTag expression but the total number of cells in the well appeared to be reduced. With 5 uM CMX001 only few weakly LTag stained cells but no agnoprotein expressing cells were observed with a more pronounced effect on total cell number. With 10 uM CMX001 no BKV-infected cell was observed and the total cell number was even more reduced. The conclusion is that CMX001 reduced the expression of early and late BKV proteins and the production of extracellular progeny but also seemed to affect the proliferation rate of RPTECs at higher concentrations.

(2) Effects of CMX001 on RPTECs (DNA Replication and Metabolic Activity)

Inspection of RPTECs by phase contrast microscopy did not reveal any signs of impaired viability during the 3 day exposure to CMX001 0.31 μM. To use more sensitive assays, host cell DNA replication and metabolic activity using BrdU incorporation and WST-1 assays in uninfected RPTECs were used. Addition of CMX001 reduced both DNA replication (FIG. 2a) and metabolic activity (FIG. 2b) of uninfected RPTECs in a concentration-dependent manner. Compared to untreated RPTECs, CMX001 at 0.08 to 10 μM decreased DNA replication by 15% to 93% and the metabolic activity by 41% to 88%, respectively. At the concentration of 0.31 μM which caused a 90% inhibition of BKV replication, it is observed an approximately 20% reduction in BrdU incorporation and WST-1 activity.

(3) Effect of CMX001 on BKV Genome Replication

To investigate whether the BKV genome replication was affected by CMX001, intracellular BKV load at 24-72 h p.i. by qPCR was measured. The intracellular BKV load was normalized to the cell number using the aspartoacylase (ACY) gene as described (See Bernhoff et al., 2008; Randhawa, et al., Quantitation of DNA of polyomaviruses BK and JC in human kidneys. J Infect Dis., 192, 504-509(2005)). Compared to untreated RPTECs, CMX001 0.31 μM reduced the intracellular BKV load by 94% at 48 h and 63% at 72 h p.i. (FIG. 3). Thus, a significant inhibitory effect of CMX001 on intracellular BKV genome replication, which is the second step of the BKV lifecycle that may be identified. This step is known to require LTag expression which also increases viral late gene expression by two mechanisms: 1. increasing the DNA templates for late gene transcription and 2. by activating transcription from the late promoter (Cole, C. N., Polyomavirinae; The Viruses and Their Replication. In Fields Virology, Third edn, pp. 1997-2043. Edited by B. N. Fields, D. M. Knipe & P. H. Howley. New York: Lippincott-Raven (1996).)

(4) CMX001's Effects on BKV Early and Late Gene Expression

To investigate expression of LTag at the single-cell level, immunofluorescence staining at 48 and 72 h p.i was performed. At 48 h p.i. the number of BKV positive cells was almost the same in CMX001 and untreated wells. At 72 h p.i., the CMX001 treated cells seemed to express less LTag per cell (FIG. 4a). When late protein expression at 48 and 72 h p.i was examined by immunofluorescence staining, a significant reduction of agnoprotein (FIG. 4a) and VP1 (data not shown) was observed in CMX001-treated RPTECs. By western blotting the decrease of VP1 was found to be 86% and 63% at 48 and 72 h p.i., respectively (FIG. 4b) while LTag staining was found to be 33% and 30% reduced at 48 and 72 h p.i., respectively. Interestingly, immunofluorescence also revealed some refractory cells in the CMX001 treated culture expressing agnoprotein at levels comparable to untreated cells even with CMX001 concentrations up to 2.5 μM. It was concluded that CMX001 significantly reduces late protein expression but also inhibit early protein expression at late time points of infection.

(5) Timecourse of CMX001 on Extracellular BKV Load

To examine the effect of CMX001 on BKV progeny over time, supernatants of treated and untreated cells were harvested at the indicated timepoints. As earlier described (Bernhoff et al., 2008), the completion of the first lifecycle of BKV(Dunlop) in untreated RPTECs take between 48 and 72 h. While an increased BKV load was observed in supernatants from untreated cells at 48 h p.i, only input virus could be detected in CMX001 treated cells 48 h p.i. At 72 h an increased viral load was seen in both untreated and CMX001 treated cells but the BKV loads in supernatants from CMX001 treated cells were 84% lower (1.13×10⁸ Geq/ml) than in untreated cells (FIG. 5). It is concluded that progeny production in CMX001 treated RPTECs may be delayed.

(6) Treatment of RPTECs Before Infection

To investigate whether or not pre-treatment of cells before virus inoculation could inhibit BKV-infection, RPTECs were either treated for 4 hours and CMX001 was replaced by complete growth medium 20 h pre-infection, or cells were treated for 23 hours at 24 h pre-infection but CMX001 was replaced at one hour before infection with complete growth medium. While treatment for 4 hours, ending 20 hours before infection, had hardly any effect on the BKV load 72 h p.i., treatment for 23 hours until one hour before infection did reduce the viral load by about 50% (FIG. 6). Thus, CMX001 pre-treatment does reduce but not prevent BKV replication.

(7) Stability of CMX001

To examine the stability of CMX001 stock solution 1 mg/ml was put in 4 or −20° C. for one week then diluted to 0.31 uM and tested for its antiviral effect by measuring the extracellular BKV load in BKV-infected RPTEC 3 d p.i. Drug stored at 4° C. had less than 60% activity while drug stored at −20° C. had an approximately 90% activity compared to the freshly prepared drug (FIG. 7).

C. Discussion

The preliminary results from treating BKV-infected RPTECs with CMX001 were shown in FIG. 1-7 as well as the above discussion related to FIGS. 1-7. CMX001 inhibits BKV-infection at the level of BKV genome replication at about 400 times lower concentrations than CDV (CDV 40 ug/ml=127 uM versus CMX001 0.31 uM). CMX001 at a concentration of 0.31 uM reduced extracellular BKV loads by approximately 90% defining the IC90. The same CMX001 concentration decreased cellular DNA replication in uninfected cells by 22% and metabolic activity by 20%. However, in some previous research (Bernhoff et al., 2008) it is shown that BKV infection increase cellular DNA replication by about 40% and metabolic activity around 20% and therefore the hypothesis is that both DNA replication and metabolic activity will be at the level of uninfected cells when CMX001 0.31 uM is used to treat BKV-infected cells.

CMX001 at 0.31 uM reduce BKV DNA replication by 94% at 48 h p.i. At the same time VP1 expression is 86% reduced. However at 72 h p.i., the decrease in DNA replication is only 63%. This discrepancy requires further studies including the effect on infectious supernatants.

When extracellular BKV loads were measured at 24, 48, and 72 h p.i., only input was detectable in CMX001 treated cells at 48 h p.i. indicating that very little or no virus is released before 48 h p.i. At 72 h p.i, only a minor increase in the BKV load was observed accounting for a 84% reduction compared to untreated cells.

Experiments to examine the effect of pre-treatment of RPTEC with CMX001 prior to infection could prevent BKV replication were conducted and results are shown above. Pre-treatment for 4 h 24 h pre-infection did not inhibit BKV replication. However, pre-treatment for 24 h until one hour before infection reduced the viral load about 50%. Thus pre-treatment of cells will partly inhibit BKV replication.

Comparing the immunofluoresence staining of CMX001 IC₉₀ treated cells 72 h p.i and CDV IC₉₀ treated cells (Bernhoff et al., 2008), LTag expression 72h p.i. seem to be more reduced by CMX001 than by CDV. As for CDV, treatment refractory cells were present even at CMX001 concentrations 8 times higher than IC₉₀. Since CMX001 does not depend on the organic anion transporter, selective expression of this transporter in the cells cannot explain the phenomena.

For each CMX001 experiment, fresh stock solutions were prepared. This could lead to minor concentration variation from experiment to experiment. The effect of storing CMX001 stock solutions was therefore tested. Storage of CMX001 at one week at 4° C. or at −20° C. decreased the antiviral activity. However, the possibility that different activity of the stored and freshly prepared CMX001 could be due to minor concentration differences in the stock cannot be excluded.

The conclusion is that the IC₉₀ of CMX001 against BK virus replication in primary human renal proximal tubule epithelial cells was 0.31 μM. In uninfected cells, CMX001 at 0.31 μM inhibited metabolic activity and DNA replication by approximately 20%.

In addition, CMX001 like CDV inhibits BKV replication in primary human RPTECs downstream of initial LTag expression. Probably due to a more favourable uptake, the IC₉₀ for CMX001 in RPTERCS is 410 times lower than for CDV. The host cell toxicity seems to be comparable to CDV. A clear advantage of CMX001 in BKV treatment is the possible oral administration.

### Example 5

### Inhibition of Polyomavirus JC Replication by CMX001

A. Material and Methods

(1) Cell Culture

COS-7 cells were grown in DMEM-5%. Astrocytes derived from progenitor cells were maintained in MEM-E-10% supplemented with Gentamycin. JCV Mad-4 (ATCC VR-1583) supernatants from infected COS-7 cells with a TCID50 of 104.5 per ml were used for infection of cultured cells.

(2) Infection and CDV-Treatment

COS-7 or astrocyte cells were infected at a confluence of 60-70% with JCV(Mad-4) at an estimated TCID50 of 0.2. After 2 h incubation at 37° C., supernatants were replaced with fresh medium without or with increasing concentration of CMX001. CMX001 was freshly dissolved to 1 mg/ml in methanol/water/ammonium hydroxide (50/50/2) and then further diluted the respective growth medium.

(3) Transfection of JCV Genome

Religated JCV Mad-4 DNA was transfected into 50% to 70% confluent COS-7 cells by using Lipofectamine 2000 (Invitrogen) according to the manufacturer's instructions at a DNA:Lipid ratio of 0.8:1.

(4) Immunofluorescence

Cells were fixed with 4% p-formaldehyde (PFA) in phosphate-buffered saline, pH 6.8 (PBS) at room temperature for 20 min and permeabilized with 0.2% Triton X-100 in PBS at room temperature for 10 min. After washing twice with PBS at room temperature for 5 min, PFA was quenched with 0.5 M NH4Cl in PBS at room temperature for 7 min followed washing twice with PBS at room temperature for 5 min. Blocking of unspecific binding sites was done with 3% milk in PBS at 37° C. for 15 min. Primary antibody (rabbit anti-VP1 1:300 in 3% milk PBS) was incubated at 37° C. for 45-60 min. Cells were washed twice with PBS on a shaker at room temperature for 5 min. Secondary antibody (chicken anti-rabbit Cy3 1:2000) and 5 μg/ml Höchst 33342 dye to stain DNA was given to the cells then incubated at 37° C. for 45-60 min. Cells were washed twice with PBS as before. Coverslips were mounted in NPropylgallat.

(5) Real Time PCR

JCV loads were quantified after DNA extraction from 100 ul cell culture supernatants and the Corbett X-tractor Gene and the Corbett VX reagents (Qiagen, Hombrechtikon, Switzerland). The real-time PCR protocol for detection of JCV DNA samples targets the JCV large T coding sequence and has been described elsewhere (5).

(6) WST-1 Assay

The metabolic activity was monitored by the colorimetric WST-1 assay (Roche) of the mitochondrial dehydrogenases in viable cells. COS-7 cells were seeded in 96 well plates and CMX001 was added at indicated concentrations. The WST-1 cleavage product was measured at 450 nm (sample) and at 650 nm (background). WST-1 plus medium alone served as blank.

(7) BrdU Assay

DNA synthesis was quantified by the colorimetric measurement of BrdU incorporation into DNA in proliferating cells using the ‘Cell proliferation ELISA, BrdU’ kit (Roche). COS-7 cells were seeded in 96 well plates and CMX001 was added at indicated concentrations. Absorbance at 450 nm (sample) and at 650 nm (background) was determined 2 h after addition of the substrate.

B. Experiments and Results

(1) Replication of JCV Mad-4 in Cell Culture

The replication characteristics of JCV Mad-4 in COS-7 and astrocyte cell cultures were firstly investigated. At 7 days post-infection (d.p.i.), JCV-infected COS-7 cells were fixed and stained by indirect immunofluorescence. As shown in FIG. 8, JCV late viral capsid protein VP1 is detectable as red signal suggesting that JCV is completing the viral life cycle in COS-7 cells (FIG. 8, left panel). The counter stain for DNA with Höchst-33342 marked the nuclei in blue (FIG. 8, middle panel). Merging both pictures (FIG. 8, right panel) indicated that JCV Mad-4 VP1 is present in the nucleus of the infected COS-7 cells. At a higher magnification, the VP1 signal was dispersed throughout the entire nucleus, but sparing the nucleoli (FIG. 8, left panel). Cells showing an intense VP1 signal in the nucleus had a diffuse staining pattern in the cytoplasm as well. JCV-infected cells showed enlarged nuclei (FIG. 8, middle panel) compared to uninfected cells present in the same cell culture (FIG. 8, right panel). The data demonstrate that COS-7 cells are susceptible to JCV Mad-4 infection and that about 30% of cells have entered the late phase of the JCV lifecycle at 7 d.p.i.

Similar experiments infecting astrocyte cells with JCV Mad-4 were performed. At 7 d.p.i, the subcellular distribution of JCV VP1 appeared similar to JCV Mad-4 infected COS-7 cells (FIG. 9, left panel, red). The counterstain for DNA with Höchst-33342 marked the astrocyte cell nuclei in blue (FIG. 9, middle panel). Merging both pictures indicated that JCV-Mad4 VP1 is present in the nucleus of the infected astrocyte cells. Comparison with the JCV Mad-4 infection of COS-7 cells, significantly fewer astrocyte cells were positive for the late protein VP1 (FIG. 9, right panel). At higher magnification, the VP1 signal was found mainly in the nucleus sparing the nucleoli and a rather diffuse pattern in the cytoplasm of the astrocyte cells (FIG. 9, left panel). Staining of the DNA indicated that large nuclei are present in the culture (FIG. 9, middle panel), which belong to JCV-infected cells (FIG. 9, right panel). Astrocyte cells were also stained for the viral early protein large T-antigen (LT) as expected, the LT was located in the nuclei of infected cells (data not shown). All cells positive for late protein VP1 also expressed LT, but few astrocyte cells were only positive for LT. This observation indicated that JCV Mad-4 proceeded through the polyomavirus life cycle as expected. The confluency at 7 days p.i indicated that astrocytes are slow growing cells and, thus, JCV replication is prolonged compared to BKV replication in human renal proximal tubular epithelial cells. Our observations suggest that a 7 day growth period is not optimal for efficient JCV replication.

Given the slower replication rate of JCV Mad-4 in astrocyte cells, the state of infection at 14 d.p.i. was examined. The results indicated that the number of cells positive for the early LTag (data not shown) and late VP1 had increased by approximately 4-fold (FIG. 10). Thus, the astrocyte cells supported JCV Mad-4 replication, but the life cycle seemed to be considerably prolonged compared to the one observed in COS-7 cells.

Next, the replication competence of religated JCV Mad-4 DNA transfected into COS-7 cells was tested. This approach would be a helpful tool to perform reverse genetics to test CMX001 efficacy on JCV variants. As shown in FIG. 11, JCV late viral capsid protein VP1 is detectable as red signal indicating that JCV is replication competent in COS-7 cells after 7 days post transfection, d.p.t. (FIG. 11, left panel). The counterstain with Höchst 33342 dye for DNA marked the nuclei in blue (FIG. 11, middle panel). Merging both pictures (FIG. 11, right panel) indicated that JCV Mad-4 VP1 is present in the nucleus of the transfected COS-7 cells. At a higher magnification, the VP1 signal was dispersed throughout the entire nucleus, but sparing the nucleoli (FIG. 11, left panel). Cells showing an intense VP1 signal in the nucleus had a diffuse staining pattern in the cytoplasm as well. JCV-transfected cells showed enlarged nuclei (FIG. 11, middle panel) compared to normal cells present in the same cell culture (FIG. 11, right panel). The data demonstrate that COS-7 cells are susceptible to JCV Mad-4 DNA transfection and that about 15% of cells have entered the late phase of the JCV lifecycle by day 7 p.i. After transfection, the subcellular staining pattern for late protein VP1 was identical to the VP1 staining after infection with JCV Mad-4.

(2) Determination of Inhibitory Concentration IC₉₀

To investigate the effect of CMX001 on JCV progeny, increasing concentrations of CMX001 were added at 2 h p.i. and supernatants harvested at 5 d.p.i. It was observed that CMX001 reduced the extracellular JCV load in a concentration dependent manner (FIG. 12). Between day 1 and 5 p.i., the viral load increased in untreated cells by about 2.5 log (1.24×10⁷ vs 5.09×10⁹). By contrast, in cells treated with 2.5 μM CMX001, it is observed only 2½-fold increase during the same time period (9.69×10⁶ vs 2.44×10⁷). Substracting the viral input defined as the viral load at day 1 p.i., CMX001 at 0.15 μM reduced the JCV viral load by >50% and 0.6 μM reduced JCV by >90%, defining the inhibitory concentration IC-₅₀ and IC-₉₀ at day 5 d.p.i., respectively.

(3) Effects of CMX001 on COS-7 Cell Metabolic Activity

Inspection of COS-7 by phase contrast microscopy did not reveal any signs of impaired viability during the 7 day exposure to CMX001 0.6 μM. To use a more sensitive assay, the effects on the cellular metabolic activity using WST-1 assay and BrdU incorporation in uninfected COS-7 cells was investigated. Addition of CMX001 reduced the metabolic activity (FIG. 13) and DNA replication (FIG. 14) of uninfected COS-7 cells in a concentration-dependent manner. Compared to untreated COS-7 cells, increasing CMX001 concentrations from 0.08 to 10 μM decreased the metabolic activity from 3% to 52% and DNA replication 83% to 10%, respectively. At 0.6 μM, COS-7 cells showed a modest loss of metabolic activity of 17%, an approximately 50% reduced BrdU incorporation. Taken together, CMX001 significantly reduced host cell metabolic activity and DNA replication at higher concentrations. Similar experiments were also conducted with astrocyte cells at CMX001 concentrations of 0.08 to 5 μM. DNA replication in uninfected astrocytes decreased by 25% to 92% at the highest CMX001 concentration (data not shown). Comparing the CMX001 associated inhibition of DNA replication both cell types, it seemed that COS-7 were slightly less sensitive (83% vs 92%, respectively). However, for astrocyte cells, the 2 h substrate incubation period of the assay seemed to be not optimal since the optical density was low compared to the readings for COS-7. This is consistent with our observation that astrocyte cells had a slower metabolism compared to COS-7 cells.

(4) CMX001 Effects on Extracellular JCV Load in COS-7

To examine the effect of CMX001 on JCV progeny levels over time, supernatants of treated and untreated cells were harvested at the indicated time points. Supernatants at day 1 p.i. were taken as a measure of input virus. In untreated cells, the extracellular JCV load increased by more than 3 logs over the observation period of 7 days (1.24×10⁷ vs 5.67×10¹⁰ geq/ml). In the presence of 5 μM CMX001, it is observed only a modest change in the JCV load of less than 1 log (1.34×10⁷ vs 8.85×10⁷ geq/ml (FIG. 15). When JC viral loads were compared for CMX001 5 uM treated cells at 7 d.p.i. with virus progeny in untreated cells at 3 d.p.i., the viral load in the CMX001 treated cells reached only 91% of the (9.72×10⁷ vs 8.85×10⁷ geq/ml). It was concluded that progeny production in CMX001 treated COS-7 cells may be delayed.

(5) CMX001 Reduces JCV Late Gene Expression

To investigate the effect of CMX001 on late gene expression, immunofluorescence staining was performed for JCV VP1. At 7 d.p.i., untreated COS-7 cells were stained for JCV VP1 as comparison (FIG. 16, upper panel). Addition of CMX001 at 1.25 μM was associated with a significant reduction of JCV VP1 signal (FIG. 16, middle panel). At the highest CMX001 concentration of 5 μM, essentially no VP1 positive cells could be observed by immunofluorescense (FIG. 16, lower panel). It was concluded that CMX001 significantly reduces JCV late protein expression between 1.25 μM and 5 μM.

(6) CMX001 Effects on Extracellular JCV Load in Astrocyte Cells

To examine the effect of CMX001 on JCV progeny levels over time, supernatants of treated and untreated cells were harvested at the indicated timepoints. To determine input virus samples were taken at 1 d.p.i. In the course of the infection for untreated cells, the extracellular JCV load changes approximately 1 log over the period of 7 days (3.22×10⁷ vs 2.30×10⁸ geq/ml). In the presence of CMX001, JCV load of less than 1 log was seen (1.34×10⁷ vs 8.85×10⁷ geq/ml (FIG. 17). It was concluded that JCV replication was significantly slower in astrocyte cells than in COS-7. Despite the tendency of low concentrations of CMX001 to inhibit progeny production, the observation period of 7 days did not allow to measure inhibitory effects of CMX001.

C. Discussion

The preliminary results suggest that CMX001 inhibits JCV replication in COS-7 cells. The CMX001 concentration of 0.6 μM reduced extracellular JCV loads by approximately 90%. This concentration is 2 orders of magnitude lower than concentrations reported for CDV inhibition, but in the same range as observed for BKV. Here, the CMX001 IC-90 of BKV replication was determined as 0.31 μM in primary tubular epithelial cells (34). It was observed that CMX001 decreased the host cell metabolic activity by 17% and DNA replication by about 50%. When extracellular JCV loads were measured from 1 to 7 d.p.i., the JCV load from cells treated with the highest concentration of CMX001 (5 μM) was only slightly higher at 5 d.p.i. than input virus at 1 d.p.i. indicating that very little or no virus is released within 4 days. At 7 d.p.i, there was only a minor increase in the JCV load at this concentration, and the level of progeny virus was below the viral load measured at 3 d.p.i. of untreated cells. In astrocyte cells, there is a trend that low concentration of CMX001 might delay the accumulation of progeny virus. However, the inefficient progeny virus production in untreated astrocyte cells 7 d.p.i demonstrated that the JCV replication cycle is significantly slower.

The difference between COS-7 and astrocyte cells is likely due to the transformed phenotype of COS-7 cells including the expression of the SV40 large T-antigen supporting a more efficient replication cycle of JCV. In astrocyte cells, this is considerably slower.

The conclusion is that the IC₅₀ and IC₉₀ values for CMX001 against JC virus replication in vitro in COS-7 cells was 0.15 and 0.6 μM, respectively. In uninfected COS-7 cells, the IC₅₀ of CMX001 for metabolic activity and DNA replication was approximately 5 and 0.6 μM, respectively. Notably, these cells express polyomavirus T antigen may be specifically sensitive to the effects of CMX001.

### Example 6

### Inhibition of Polyomavirus JC Replication by CMX001

### Compounds:

Test material CMX021 (cidofovir) provided by Chimerix, Inc. was solubilized at 40 mM in water and CMX001 was solubilized in DMSO at 20 mM. Test materials were evaluated using a 100 μM high test concentration for CMX-021 and 500 nM high test concentration for CMX001 with serial dilutions in half-log increments for the in vitro antiviral assay. A second assay was performed using a lowered high test concentration of 10 μM for CMX-021.

### Anti-JC Polyomavirus Assay:

### Cell Preparation

Human astrocytes (ScienCell catalog #1800) were passaged in astrocyte medium (ScienCell catalog #1801; basal medium supplemented with 2% FBS, astrocyte growth supplement, and Pen/Strep)in T-75 flasks coated with 15 μg/mL poly-L-lysine prior to use in the antiviral assay. Total cell and viability quantification was performed using a hemocytometer and Trypan Blue dye exclusion. Cell viability was greater than 95% for the cells to be utilized in the assay. The cells were resuspended at 1×10⁶ cells per ml in astrocyte medium to the poly-L-lysine coated microtiter plates in a volume of 100 μL and allowed to adhere overnight at 37° C.

### Virus Preparation

The virus used for the assay was JCV_{MAD-4} obtained from the ATCC (catalog #VR-1583) and was grown in COS-7 cells for the production of stock virus pools. A pretitered aliquot of virus was removed from the freezer (−80° C.) and allowed to thaw slowly to room temperature in a biological safety cabinet. Virus was resuspended and diluted into tissue culture medium such that the amount of virus added to each well in a volume of 100 μL was the amount optimized by quantitative PCR at 7 days post-infection.

### Plate Format

Each plate contains cell control wells (cells only), virus control wells (cells plus virus), drug toxicity wells (cells plus drug only), drug colorimetric control wells (drug only) as well as experimental wells (drug plus cells plus virus). Samples were tested in triplicate with five half-log dilutions per compound.

### Toxicity Evaluation

Following incubation at 37° C. in a 5% CO₂ incubator, the test plates were stained with the tetrazolium dye XTT (2,3-bis(2-methoxy-4-nitro-5-sulfophenyl)-5-[(phenylamino)carbonyl]-2H-tetrazolium hydroxide). XTT-tetrazolium was metabolized by the mitochondrial enzymes of metabolically active cells to a soluble formazan product. XTT solution was prepared daily as a stock of 1 mg/mL in RPMI1640. Phenazine methosulfate (PMS) solution was prepared at 0.15 mg/mL in PBS and stored in the dark at 20° C. XTT/PMS stock was prepared immediately before use by adding 40 μL of PMS per ml of XTT solution. Fifty microliters of XTT/PMS was added to each well of the plate and the plate was reincubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader.

### Measurement of Virus Replication by Quantitative PCR

A JC virus DNA product for use as a quantitative standard was generated by PCR amplification of viral DNA extracted during titration of the virus. Briefly, 5 μL of viral DNA from the Day 7 100 μl titration specimen was amplified using TaqPro Complete PCR mix (Denville Scientific) and DNA oligonucleotides JCV3827F (5′-GGTTTCCAAGGCATACTGTGTAAC-3′) and JT-2 (5′-GAGAAGTGGGATG AAGACCTGTTT-3′). The resulting 532 base pair product was purified using QlAquick PCR purification columns and reagents (Qiagen) and quantified based on absorbance at 260 nm. The sequence of the product was verified as JC virus by automated dideoxy sequencing and blast analysis against the NCBI non redundant database.

Viral DNA was extracted from 50 μL of cell culture supernatant using MagMax AI/ND Viral RNA Isolation Kit (Ambion) according to the manufacturers recommended procedure. Isolated viral DNA was eluted in 50 μL of elution buffer provided in the kit and 5 μL of the extracted DNA was analyzed by qPCR using SYBRGreen-ER qPCR with ROX reagents (Invitrogen) and DNA oligonucleotide primers JCV3827F and JT-2 in an Applied Biosystems 7900HT Sequence Detection System. Five microliters (5 μL) of serial 10-fold dilutions of the quantitative standard ranging from 1×10⁶ copies/μL to 10 copies/μL was subjected in duplicate to qPCR analysis to establish the standard curve (FIG. 1). The quantity of viral DNA in each specimen was extrapolated from the standard curve by the SDS2.1 software integrated with the 7900HT Sequence Detection System. The results are expressed as a relative percentage of viral DNA present in the untreated virus control supernatants.

### Data Analysis

Raw data was collected from the Softmax Pro 4.6 software and imported into a Microsoft Excel 2003 spreadsheet for analysis by linear curve fit calculations.

### Results

Anti-JC Polyomavirus Evaluations: CMX001 and CMX021 (CDV) were evaluated against the MAD4 strain of JCV in human astrocytes in two experiments. Different lots of frozen astrocytes were used in the two experiments.

Cidofovir (CMX021-009) was evaluated in parallel with CMX001-044 and yielded EC₅₀ values of 0.19 and 0.57 μM with TC₅₀ values in human astrocytes of 68.11 and 1.82 μM for calculated therapeutic indices of 358.5 and 3.2 in human astrocytes. CMX001-044 yielded EC₅₀ values of 8.21 and 30.36 nM with TC₅₀ values in human astrocytes of 134.4 and 165.8 μM for calculated therapeutic indices of 16.4 and 5.5 in human astrocytes.

### Discussion

Two samples were evaluated for antiviral activity against JC Polyomavirus (JCV) using microtiter in vitro assay systems standardized by IrnQuest BioSciences, Inc. using two lots of human astrocytes. Compound CMX021-009 demonstrated antiviral activity in HA cells against JCV_{MAD-4} yielding therapeutic indices of 358.5 and 3.2 in independent assays resulting from similar EC₅₀ values but significantly different TC₅₀ values. CMX-001-044 demonstrated consistent antiviral activity from JCV infection of different HA cell lots yielding therapeutic indices of 16.4 and 5.5.

### Example 7

### Using CMX001 on Human Patients with ERV-Associated Intracranial Post-Transplant Lymphoproliferative Disorder (PTLD)

The first patient is a 11-year-old patient with a history of sickle cell anemia developed EBV-associated intracranial post-transplant lymphoproliferative disorder (PTLD). EBV was positive in the plasma (7 Dec. and 14 Dec. 2010) and brain biopsies were consistent with PTLD. In early December, the patient presented with a 3 day history of persistent headache, nausea, vomiting, and diarrhea. The patient was admitted to the hospital and had an acute episode of severe headache with possible seizure activity. A CT of the brain showed a ring-enhancing mass in the right frontal lobe and brain biopsy was consistent with EBV-associated PTLD. The patient was admitted to PICU. High intracranial pressure, repetitive seizures associated with apnea led to intubtion and emergency request for CMX001. The use of CMX001 in this patient with EBV-associated PTLD is ongoing since 26 Dec. 2009. The patient has tolerated CMX001 well, and continues to receive 4 mg/kg twice weekly. She has had clinical improvement of her signs and symptoms of disease as well as stabilization if not reduction of her intracranial mass. EBV in the plasma remains negative.

The second patient was a 6 month old heart transplant recipient with EBV-associated PTLD. The patient acquired a primary EBV infection post-operatively. PET scans showed lesions in the liver, lung, and bone (iliac crest) consistent with PTLD. The clinical condition continued to destabilize with what was presumed to be EBV-associated encephalitis with EBV detected in the CSF, clinical and EEG-correlated seizure activity and decreased responsiveness and changes in mental status. At the time of request for CMX001, the patient was on mechanical ventilation, had evidence of both pneumonia and PTLD of his lungs, evidence of seizure activity with clinical criteria for encephalopathy being present. The patient received his first dose of CMX001, 20 mg (approximately 3.3 mg/kg) on 3 Mar. 2010, and his second dose on 7 Mar. 2010 via NG tube The patient had a significant decline in EBV viral loads from 267,338 copies/mL (3 Mar. 2010) to 47,427 (7 Mar. 2010). The patient continued to show evidence of progressive neurologic injury. The parents withdrew support on the day following his second dose of CMX001.

### Clinical Studies

### Example 8

An initial study was conducted to evaluate the safety and pharmacokinetics of CMX001 in healthy volunteers. The study consisted of a single dose arm (SD) and a multiple dose arm (MD). In the single dose arm 7 cohorts of 6 subjects were treated (4 subjects received active drug and 2 placebo). Enrollment was staggered as 2 subjects (one active, one placebo) followed by 4 subjects (Groups A and B). The estimated single doses for the two highest doses treated for a 75 kg subject were 40 mg (0.6mg/kg cohort 6) and 70 mg (1 mg/kg cohort 7). In the multiple dose arm, cohort 6MD received 0.1 mg/kg on Day 0, 6 and 12; Cohort 7MD received 0.2 mg/kg on Day 0, 6 and 12. Levels of cidofovir, CMX001 and CMX064 (major metabolite) were measured in blood and urine of subjects during the course of the study. Gastrointestinal (GI) monitoring of the subjects included (a) monitoring for clinical signs of GI adverse events, (b) monitoring for clinical symptoms using a visual Analog Scale, (c) monitoring for appetite loss/anorexia, nausea, vomiting, diarrhea, constipation and intestinal gas/bloating, (d) laboratory tests for fecal occult blood; serum electrolytes, urine specific gravity, BUN/creatinine ratio; serum albumin, and lipids, and (e) diagnostic studies (the Wireless capsule endoscopy (PillCam®, Given Imaging)).

Upon the completion of the study of cohort 6 (600 μg/kg) (while still blinded) it was observed as follows:
No post-dose clinically significant gastrointestinal capsule endoscopy findings attributable to drug.
No drug associated clinically significant changes to clinical laboratory values, including those indicative of kidney dysfunction.
No serious adverse events (SAEs), no significant adverse events (AEs) (i.e. ≧Grade 2), no AEs directly attributable to drug.

Plasma concentration curves of CMX001 following a single dose administration are shown in FIG. 18, and plasma concentration curves of Cidofovir following a single dose of CMX001 are shown in FIG. 19.

Table 4 illustrates the PK comparison of CMX001 with CMX021 and CMX064 for mouse, rabbit and human.

TABLE 4

| | | CMX001 | | CMX021 | | CMX064 | |
|---|---|---|---|---|---|---|---|
| | Dose | Cmax | AUCO→ | Cmax | AUCO→ | Cmax | AUCO→ |
| Species | (mg/kg) | (ng/mL) | (ng * h/mL) | (ng/mL) | (ng * h/mL) | (ng/mL) | (ng * h/mL) |
| Mouse- | 2 | 7.9-18.0 | 83.14-102.4 | BQL- | ND-50.1 | — | — |
| Rabbit | | | | 5.44 | | | |
| Human | 0.025 | 2.36 | 18.51 | BQL | ND | 1.69 | 11.83 |
| | 0.050 | 5.63 | 36.32 | 1.51 | 33.28 | 4.63 | 38.95 |
| | 0.100 | 10.62 | 133.47 | 3.44 | 125.14 | 2.85 | 34.02 |
| | 0.200 | 24.48 | 225.49 | 5.41 | 189.92 | 4.55 | 39.73 |
| | 0.400 | 68.13 | 526.37 | 10.44 | 444.76 | 23.03 | 202.99 |
| | 0.600 | 114.73 | 728.8 | 12.19 | 519.0 | 24.86 | 187.0 |

Calculated based on mouse doses of 2 and 10 mg/kg, rabbit doses of 5 and 10 mg/kg
ND Not Determined,
BQL Below Quantitation Limit

### Example 9

A study has been conducted on patients with various JC viral infections and diseases associated with JC virus. The details of the study are summarized in the Table 5(a) below.

After these patients are treated with CMX001, the patients have shown significant improvements.

TABLE 5(a)

| | | | JCV Viral Load (copies/mL) | | | |
|---|---|---|---|---|---|---|
| Sub | TMT | | CSF | Blood | Urine | Clinical Comment |
| 1 | ~5 | wks | Pre: 306 | Pre-D32: | D7: 557 | The patient had a fatal outcome likely due to |
| | 2 | mg/kg | D32: <300 | ND | D7-EOT: ND | disease progression and pneumonia. During |
| | | | | | | treatment with CMX001, the JCV viral load |
| | | | | | | became undetectable (<300 copies/mL) in the CSF |
| | | | | | | and urine from an initial level of 398 copies/mL in |
| | | | | | | the CSF and an initial level of 557 copies/mL in |
| | | | | | | the urine. No drug-related adverse events were |
| | | | | | | reported. |
| 2 | ~4.5 | mts | | Pre: 3456 | | During treatment, the patient regained swallowing |
| | 4 | mg/kg | | EOT: ND | | function and was able to eat solid foods. She was |
| | | | | | | relearning speech with difficulty. Dexterity of left |
| | | | | | | hand was improved, but she did not fully regained |
| | | | | | | fine motor functions. Right sided paralysis was |
| | | | | | | unchanged. MRI of the brain showed no new |
| | | | | | | lesions. The old PML lesions were slightly smaller |
| | | | | | | and appear chronic. |
| 3 | 4 | days | Pre: 518 | No data | Pre: <500 | During treatment, the patient had improvement in |
| | (2 doses) | | Post: <500 | | Post: no data | his neurologic condition. The duration of |
| | 4 | mg/kg | | | | therapy was insufficient for assessment of a complete |
| | | | | | | virologic response, although JCV in CSF became |
| | | | | | | undetectable at the single post-therapy time point. |
| | | | | | | The patient experienced a recurrence of his HCV |
| | | | | | | and the family elected to withdraw care. |
| 4 | ~2.5 | wks | Pre: 16,474 | Pre: 148 | Pre: 43 | During treatment, the patient started to move his |
| | 4 | mg/kg | EOT: 1070 | EOT: 261 | Post: ND | right arm and leg after having been completely |
| | | | | | | plegic. Reports indicated there may also be some |
| | | | | | | corresponding changes in the grammatical structure |
| | | | | | | of his speech (adding back prepositions) which |
| | | | | | | may signal the resolution of his frontal operculum |
| | | | | | | syndrome. The physician indicated the patient has |
| | | | | | | that new look . . . the one that signals hope. The |
| | | | | | | physician also said that such improvements in |
| | | | | | | cases of PML can only be considered possible, yet |
| | | | | | | highly unlikely. |
| 5 | Ongoing | | Pre: | Pre: 1800 | No Data | Patient was status epilepticus at start treatment, |
| | ~3 | wks | 168,225 | D7 500 | | rapid with improvement leading to extubation after |
| | 4 | mg/kg | | | | 48 h. One CMX001 dose skipped due to concern |

related to increased liver enzymes, in parallel
clinical impression of neurological worsening.
Negative reintroduction with persistently normal
liver enzymes. Patient was transferred to rehab
after 3 weeks CMX001, and is slowly improving.

ND: not detected or undetectable
EOT: end of treatment, day of last dose or the 1^{st} VL following the last dose
No data: JCV DNA was not assessed in this specimen type or at this time point

### Example 10

A further study has been conducted on patients with various viral infections and diseases associated with virus. The details of the study are summarized in the Table 5 below.

As shown in Table 5(b), all patients that have been previously treated with other antiviral agents for example cidofovir. However, the patents did not respond well to these medications. After these patients are treated with CMX001, the patients show significant improvements.

TABLE 5(b)

| | Patient | | CMX001 | Previous | Concomitant | |
|---|---|---|---|---|---|---|
| | Demographics | Viral | Dose | Antiviral | Antiviral | Virology/Clinical |
| EIND # | (age & weight) | Infection | Regimen | Medications | Medications | Data |
| 107275 | 29 yrs/69 kg | BKV | 2 mg/kg first | Acyclovir | None reported | CMV suppression |
| | | hemorrhagic | dose; | (ACV), | | continued during |
| | | cystitis | 1 mg/kg | leflunomide, | | CMX001 therapy, |
| | | CMV | second dose; | ganciclovir | | after discontinuation |
| | | | twice weekly | (GCV) | | of ganciclovir. BK |
| | | | 2 mg/kg | | | viruria transiently |
| | | | thereafter, | | | decreased during |
| | | | five doses | | | administration of |
| | | | | | | CMX001. |
| | | | | | | Hemorrhagic |
| | | | | | | cystitis clinically |
| | | | | | | improved while on |
| | | | | | | CMX001 |
| 107640 | 66 yrs/108 kg | ADV | 3 mg/kg first | ACV, | None reported | AdV 35 (urine). 1 |
| | (edema), ~80 kg | nephritis | dose; | Valtrex, | | to 2 log reductions |
| | (normal) | and viremia | 2.5 mg/kg | Cidifovir | | in adenovirus |
| | | BKV | twice weekly | (CDV) | | viremia, ADV |
| | | | One month | | | viruria, CMV |
| | | | | | | viremia, BKV |
| | | | | | | viremia and BKV |
| | | | | | | viruria were |
| | | | | | | observed. |
| | | | | | | BK viremia |
| | | | | | | decreased from |
| | | | | | | 1900 to 28 |
| | | | | | | copies/mL; BK |
| | | | | | | viruria declined |
| | | | | | | from 120 million to |
| | | | | | | 48 million |
| | | | | | | copies/mL |
| 108104 | 20 yrs/67 kg | ADV | 2 mg/kg | ACV, | None reported | AdV 34 (plasma). |
| | | BKV | twice weekly | CDV, | | AdV plasma viremia |
| | | CMV | 4 mg/kg | GCV | | declined 3.1 logs |
| | | | twice weekly | | | from 5.1 log₁₀ to the |
| | | | 2 months | | | limit of detection; |
| | | | | | | AdV viruria |
| | | | | | | appeared to respond |
| | | | | | | well initially with a |
| | | | | | | 2.8 log₁₀ decline, but |
| | | | | | | it rebounded to |
| | | | | | | within 0.7 log₁₀ |
| | | | | | | from baseline. |
| | | | | | | Similar trends may |
| | | | | | | be occurring with |
| | | | | | | BKV and CMV. |
| | | | | | | Notably, this patient |
| | | | | | | had initially normal |
| | | | | | | exposures to |
| | | | | | | CMX001 and then |
| | | | | | | low exposures that |
| | | | | | | may correlate with |
| | | | | | | these responses. |
| | | | | | | Renal function |
| | | | | | | improved on therapy |
| | | | | | | and hemorrhagic |
| | | | | | | cystitis improved. |
| 108897 | 47 yrs/89.8 kg | BKV | 4 mg/kg | Valtrex, | None reported | BKV in urine |
| | | | twice weekly | CDV | | decreased from 7.7 |
| | | | 2 weeks | | | log10 at baseline to |
| | | | | | | 5.1 log10 at last |
| | | | | | | dose. Notably, |
| | | | | | | baseline was 18 |
| | | | | | | days prior to first |
| | | | | | | dose and a four-fold |
| | | | | | | variance in viral |
| | | | | | | load was seen |
| | | | | | | between two |
| | | | | | | measures taken on |
| | | | | | | the same day. |
| | | | | | | Hemorragic cystitis |
| | | | | | | resolved |
| 109232 | 53 yrs/95.5 kg | BKV | 2 mg/kg | CDV | None reported | BKV in urine |
| | | | twice weekly | | | decreased 2.2 log10 |
| | | | | | | over the first two |
| | | | | | | weeks of CMX001 |
| | | | | | | therapy; data for |
| | | | | | | later timepoints |
| | | | | | | pending. |

### Example 11

### CMX001 and Adenovirus Infection

Adenovirus infection causes severe morbidity and mortality in immunocompromised patients. There are currently no FDA approved therapies for treatment of adenovirus infections in the United States, with only anecdotal, off-label uses described for a variety of anti-viral agents or immune therapies such as WIG or donor-lymphocyte infusion. The use of many of these agents is limited by toxicity with little evidence of efficacy. We report the successful use of CMX001 as a novel anti-viral agent in the treatment of a case of severe, disseminated adenovirus infection in a pediatric bone marrow transplant recipient. Despite oral administration in the presence of severe GI dysfunction due to graft-versus-host disease and biopsy proven viral infection of the colon, the patient demonstrated drug absorption followed by clinical and virologic (8 log decrease in viral load) response to treatment.

Infections are a major cause of morbidity and mortality following hematopoietic stem cell transplantation (HSCT). Due to the expansion of available agents to treat bacterial and fungal infection as well as changes in the approach to HSCT such as the use of lymphocyte-targeted conditioning, umbilical cord blood as a stem cell source and T cell depleted allografts, viral infections are emerging as one of the major challenges in the field. Management of cytomegalovirus (CMV) reactivation using high-sensitivity monitoring with PCR and prophylactic use of effective antiviral agents has reduced the incidence of CMV disease. However, other viral pathogens such as adenovirus remain a major cause of morbidity and mortality, in part, due to lack of effective agents. Although Vistide® (cidofovir injection) is used to treat adenovirus infection it has limited clinical utility due to its potential to cause nephrotoxicity.

CMX001 is an orally available lipid-conjugate of the nucleoside analog, cidofovir. The lipid conjugate allows oral administration and enables rapid uptake of CMX001 into cells where it is cleaved and the resulting cidofovir is phosphorylated to the active antiviral agent. CMX001 has a broad spectrum of activity, effective against all 5 families of double-stranded DNA viruses including orthopoxviruses, [variola, monkeypox (MPXV), vaccinia (VACV), cowpox (CPXV), and ectromelia (ECTV) viruses], herpesviruses [cytomegalovirus (CMV), herpes simplex (HSV) 1 and -2, varicella zoster (VZV), Epstein-Barr (EBV), and human herpes (HHV-6, and HHV-8) viruses], adenoviruses (AdV), polyomaviruses [BK virus], and papilloma viruses. The antiviral activity of CMX001 against adenovirus has been characterized in vitro in cell culture systems and in vivo in animal models. In vitro studies demonstrated that CMX001 is effective against multiple serotypes of adenovirus. The majority of serotypes have EC₅₀s <50 nM with the exception of AdV 31 (EC₅₀ of 0.28 μM). Compared to cidofovir, CMX001 is 33- to 200-fold more potent against AdV types 3, 5, 7 and 8, and 5-fold more activity against AdV 31. In vivo, CMX001 was highly effective against adenovirus in an immunocompromised, AdV 5 Syrian Hamster model characterized by severe systemic disease with hepatic necrosis. CMX001 (2.5 mg/kg/d) prevented mortality in AdV 5-infected hamsters when administered two days post-infection. Infectious AdV5 titers in liver were reduced 6 logs to nearly undetectable levels in most animals by seven days post infection. Here we report the first case of successful eradication of disseminated adenovirus by CMX001 in a severely immunocompromised pediatric recipient following failure to respond to cidofovir.

### Clinical Course

A 12 year old girl with severe aplastic anemia received a 9/10 HLA matched (allele mismatch at B) allogeneic bone marrow transplantation following alemtuzumab/fludarabine/melphalan conditioning. Despite prophylaxis with cyclosporine A, methotrexate and anti-thymocyte globulin she developed grade 4 graft-versus-host disease (GvHD) of the intestine and skin that was refractory to high-dose steroids and monoclonal antibodies. She subsequently received mesenchymal stem cells on a compassionate use experimental protocol with gradual improvement, such that she tolerated enteral feeding. She developed recurrent diarrhea in association with rising quantitative plasma adenovirus DNA PCR analyzed using a commercially available, validated assay (Viracor). Colonic biopsies and stool cultures confirmed adenovirus enteritis, with pulmonary specimens also culture positive later in the disease course. Despite taper of immunosuppression (tacrolimus, mycophenolate, and prednisone) and treatment with IV cidofovir 1 mg/kg 3 times weekly, plasma adenoviremia increased to 680 million copies/ml. Her clinical condition deteriorated with severe gastrointestinal (GI) bleeding, hepatitis and, eventually respiratory failure despite increasing cidofovir to 5 mg/kg once weekly and administration of intravenous immune globulin.

Her renal function deteriorated while on Vistide®, eventually necessitating veno-venous hemodialysis. Given the extremely poor prognosis for disseminated adenovirus infection in this setting, CMX001 was administered under an FDA-approved Emergency Investigational New Drug Application (EIND) following IRB approval and appropriate informed consent by the parent. At the time of treatment with CMX001, the patient was intubated and sedated, had metabolic acidosis, and renal insufficiency with creatinine levels between 1.6 and 1.9. The patient had unremitting gastrointestinal bleeding requiring daily transfusions of packed RBCs and platelets. CMX001 was started at a dose of 2 mg/kg twice weekly with a prompt and continued reduction in plasma adenovirus load noted following initiation of therapy (FIG. 20). Within the first 5 weeks of CMX001 therapy, transfusion requirements dramatically decreased, renal function and hepatic function improved and the patient was extubated, with an undetectable viral load. Within 8 weeks, hemodialysis was discontinued, the patient was transferred from the ICU with resolution of GI bleeding and renal impairment. The patient had persistent absolute lymphopenia counts (ALC) less than 300 throughout the treatment course. The viral load showed a marked reduction while on therapy, despite the fact that the ALC remained well below normal limits. There was subsequent recovery of ALC after the viral load had decreased to near undetectable. Following resolution of AdV viremia and clinical signs and symptoms of disease, the patient was maintained on CMX001 at a dose of 3 mg/kg weekly. CMX001 was well tolerated and no drug-related serious adverse events were observed.

### CMX001 Administration and Pharmacokinetics

The patient initially required continuous nasogastric (NG) suctioning due to severe GI bleeding, thus CMX001 was administered via NG tube with interruption of suctioning for as long as tolerated (generally 1-3 hours). Plasma samples were obtained at regular intervals throughout her treatment course for analysis of CMX001 and cidofovir concentrations using a validated analytical method (LC/MS/MS). Interestingly, AdV viremia resolved despite lower than predicted plasma exposure to CMX001 during the first 5 weeks of treatment (through about the 10^{th} dose) (FIG. 20, inset). The potential impact of the patients GI bleeding and use of nasogastric suctioning on absorption, and ultimately systemic exposure to CMX001 are uncertain but likely decreased exposure because as the patient's GI bleeding resolved, higher systemic exposures to CMX001 were observed. Following elimination of residual cidofovir from the preceding administration of Vistide®, maximum plasma concentrations of CMX001-derived cidofovir never exceeded 80 ng/mL. By contrast, peak plasma concentrations of cidofovir after administration of Vistide (5 mg/kg with probenecid) are in the range of 19,600 ng/mL (Vistide® package insert).

Disseminated adenovirus is a serious and often fatal complication of HSCT. Clinical manifestations include respiratory disease, hepatitis, nephritis, cystitis, gastrointestinal disease including hemorrhagic colitis and enteritis, encephalitis, and multiorgan failure. Risk factors for disease include young age, allogeneic transplantation, T cell depleting conditioning regimens, unrelated or HLA-mismatched grafts, lymphocytopenia, and GvHD. The expected mortality rate in patients with disseminated adenovirus disease is up to 80% depending on the organ system involved. There are currently no FDA-approved therapies for adenovirus infection. While cidofovir is often used in this setting, efficacy has not been well established due to virulence of the virus, variable pharmacokinetics/dynamics, and unavoidable toxicities of prolonged cidofovir therapy. Thus, new agents to treat adenovirus following HSCT are clearly needed. Furthermore, the availability of high-sensitivity PCR-based monitoring offers the opportunity to monitor and potentially prevent disseminated adenovirus infection utilizing pre-emptive therapeutic approaches. As is the case with CMV infection in this patient population, pre-emptive therapy is likely to be more practical and effective as less toxic agents are identified, particularly oral agents with excellent bioavailability. Our extremely high-risk patient exhibited complete response to treatment with CMX001, in combination with mesenchymal stem cell infusion and continued immunosuppression. Plasma analysis demonstrated, initially, lower than expected concentrations of CMX001 presumably due to extensive GI disease, however, a complete virologic and clinical response was observed and plasma concentrations increased as the GI disease resolved. Also notable in this patient was an improvement in renal function while receiving treatment with CMX001 which is consistent with animal and human data that have revealed no evidence of nephrotoxicity associated with CMX001. This is likely due to much lower peak plasma concentrations of cidofovir observed following administration of CMX001 compared with Vistide. In this patient peak cidofovir concentrations were more than 100-fold lower than those reported in the label for administration of 5 mg/kg Vistide®. Cidofovir in plasma is not thought to be relevant to the efficacy of CMX001, rather, it is presumed to be an elimination product. Hence low plasma concentrations of cidofovir are a desirable trait of CMX001 that reduces the potential for nephrotoxicity with no relevance to efficacy.

### Example 12

### CMX001 and Adenovirus Infections in Immunocompromised Patients

The prevalence of adenovirus (AdV) infections in immunocompromised patients is increasing, now occurring in 8.5%-30% of allogeneic BMT recipients, particularly in patients with severe Graft-Versus-Host Disease (GVHD) and absolute lymphocyte counts (ALC) <300 cells/mm³, Mortality rates are as high as 70%. Cidofovir (CDV) is used to treat AdV disease, without supportive data from prospective or controlled trials. CDV is associated with significant nephrotoxicity and occasional neutropenia. No therapeutic agent has been established as the definitive treatment for AdV infections. CMX001, a lipid conjugate of Cidofovir is taken up by the cells and cleaved intracellular to yield free CDV, which is phosphorylated to produce the active antiviral agent, cidofovir diphosphate (CDV-PP). In vitro, CMX001 yields much higher intracellular levels of CDV-PP in human peripheral blood mononuclear cells (PBMCs) than equimolar CDV. This explains the increase in potency against adenovirus shown below in Table 6. Unlike CDV which has to be administered intravenously, CMX001 is dosed orally. Also CMX001 has low potential for nephrotoxicity, probably due to the inability of the renal organic anion transporters to recognize CMX001.

TABLE 6
Comparison of In Vitro Activity of Several Antiviral
Agents against Adenovirus

| | IC₅₀(μM) | | | |
|---|---|---|---|---|
| Virus | Acyclovir | Ganciclovir | Cidofovir | CMX001 |
| Adenovirus | >100^{a} | 4.5-33^{b} | 1.3 | 0.02 |

*Cancer Gene Ther 2003 10:791
^{b}CID 2006 43:331
^{c}JID 2005191:396

### Methods

The records of patients who were granted emergency investigational-new-drug approval for CMX001 for treatment of AdV were analyzed retrospectively. Of the 16 patients with AdV disease treated with CMX001, 13 had data available for ≧4 weeks after starting CMX001. Doses of CMX001 ranged from approximately 1 mg/kg once weekly to 4 mg/kg twice weekly. Adenovirus qPCR was performed at VireCor (9 cases), Focus Diagnostics (2) and Molecular Virology Laboratory, University of Washington (2). Disseminated AdV disease was defined by the isolation of the virus from 2 or more sites, including blood. At the end of CMX001 treatment, virologic response (VR) was defined as either ≧99% drop in plasma VL from baseline or undetectable plasma virus. The Wilcoxon signed rank test was used to evaluate whether VL changed from baseline to weeks 1, 2, 4, 6, and 8. Logistic regression models were employed to evaluate possible associations between covariates and VR.

TABLE 7
Change in adenovirus viral load (log10) from initiation
of CMX001 to week 8

| | Median decrease | 95% confidence | |
|---|---|---|---|
| Week | from baseline | interval | p-value |
| 1 | 1.43 | (0.375, 2.22) | 0.002 |
| 2 | 1.71 | (0.96, 2.45) | 0.001 |
| 4 | 1.89 | (0.88, 2.84) | 0.002 |
| 6 | 2.44 | (0.78, 3.17) | 0.006 |
| 8 | 2.98 | (2.00, 5.26) | 0.004 |

### Results

### Baseline

Median age of the group was 12 years (range 0.92-66). There were 5 male and 8 female, 8 pediatric patients and 5 adults. One patient had severe combined immunodeficiency, one solid organ transplant recipient, 11 hematopoietic cell transplant recipients (10 of whom had GVHD). All 13 patients had viremia with AdV isolation from ≧1 additional site: GI 7 (53.8%); GU 4 (30.8%); lungs 3 (23.1%); brain 1 (3.85%); and bone marrow 1 (3.85%). The disease was diagnosed at a median of 68 days (15-720) after transplantation. Median ALC at diagnosis was 300 cells/μL (range 50-1500). All patients received prior CDV and were switched to CMX001 after a median of 21 days (range 7-90) due to refractory AdV infection or renal toxicity. Patients were treated with CMX001 for a median of 68 days (range 15-208).

### End of Therapy

The relationships of ALC and VL at weeks 1, 2, 4, 6 and 8, compared to baseline, are shown in FIGS. 21a-21e. VR was not associated with age, sex, total mg of CMX001 received, AUC or Cmax of CMX001. The pharmacodynamic effect of CMX001 on VL is shown in Table 7. VR was achieved in 8 (61.5%) of 13 patients. No serious adverse events, gastro-intestinal, renal or bone marrow toxicity were reported. CMX001 has potent in vitro activity against adenovirus and may be a future option for the treatment of adenoviral disease in immunocompromised patients. No significant safety issues were raised with CMX001 in this critically ill population.

### Example 13

### CMX001 for Cytomegalovirus Infections in Stem Cell Transplant Patients

Cytomegalovirus (CMV) infections are associated with significant morbidity and mortality in the stem cell transplant setting. CMX001, a lipid conjugate of cidofovir is administered orally and circulates as the lipid conjugate in plasma; it is efficiently taken up by target cells and high concentrations of the active antiviral are achieved intracellularly. The first clinical experiences in stem cell transplant patients infected with CMV who received CMX001 are described. The patients had history of AML (acute myelogenous leukemia; 3 patients), refractory lymphoma, multiple myeloma, and severe aplastic anemia, and sickle cell anemia. Six of the seven patients had received stem cell transplantation (SCT) and the seventh awaited SCT. Treatment with CMX001 was initiated pre-transplant in one patient, and 21 days to greater than 2 years in six patients (median of 61 days).

7 patients with CMV viremia who were treated under emergency IND because of lack of other reasonable therapeutic options were treated. All 7 patients had failed conventional antiviral therapy: all patients had received ganciclovir and/or valganciclovir, 6 had received foscarnet, and 4 had received CMV 1G. The doses of CMX001 in these patients ranged from 80 mg to 300 mg (approximately 2 to 4 mg/kg); follow-up data was available for at least 4 weeks in all patients. Virologic response was defined as more than a 90% reduction (1 log 10) in viral load (VL) and complete response was defined as an undetectable viral load.

The 4 males and 3 females treated had a median age of 55 years (range 11 to 69 years); they were treated with CMX001 for a median of 88 days (range 29-131 days). The median reduction in VL was greater than 1.2 log 10 at 4 weeks. A complete response was observed in ⅗ (60%) patients who did not have mutations in the CMV polymerase UL54 gene; ⅖ had an average reduction in CMV by PCR of 1.2 log 10. Neither of two patients with a relevant mutation in UL54 (L501F and A987G) had a 1 log reduction in viremia at the last time point.

Two patients had pre-existing renal insufficiency; no dose adjustment was needed based on kidney function. This is in contrast to treatment with cidofovir, which is known to be nephrotoxic. During treatment with CMX001, one patient experienced C. difficile colitis, one experienced pancytopenia along with graft versus host disease (GVHD) and sepsis, one with a seizure disorder experienced seizure, and one experienced severe GVHD. No trends were observed in serious adverse events.

### Example 14

### CMX001 and Ganciclovir Combination Therapy for Treatment of Human Cytomegalovirus Infection

CMX001 has been reported previously to inhibit the replication of human cytomegalovirus (HCMV) both in vitro and in vivo. Since CMX001 is a monophosphate analog, it does not require initial phosphorylation by the HCMV UL97 kinase; therefore, it is highly active against most ganciclovir (GCV) resistant strains, and should be useful in the treatment of resistant-virus infections. We investigated the antiviral activity of CMX001 in combination with GCV in vitro to evaluate the efficacy and safety of this combination. Human foreskin fibroblast cells were infected with HCMV at a multiplicity of infection of 0.01 PFU/cell and serial concentrations of CMX001 and GCV alone or in combination were added to either uninfected or infected cells. Total DNA was harvested following a 7 day incubation and the copy number of viral DNA was determined by real time PCR. As expected, CMX001 was highly active against HCMV and reduced the quantity of viral DNA by 10-fold at concentrations less than 1 nanomolar, and 1000-fold at 10 nanomolar. The efficacy of GCV was comparatively modest and reduced the accumulation of viral DNA by less than 10-fold at 10 μM. Combinations of CMX001 and GCV were synergistic, when concentrations of CMX001 as low as 3 picomolar were added to GCV. No significant changes in cytotoxicity were observed for any of the concentrations tested confirming that the combination was not toxic. The exceptional potency of CMX001 observed in these assays was confirmed in a quantitative real-time RT-PCR-based array that determined levels of all viral transcripts, Reductions in the levels of viral transcripts were consistent with the reductions in genome copy number and reflected the marked inhibition of viral replication in vitro relative to GCV. These results clearly indicated that combinations using suboptimal concentrations of CMX001 with GCV are synergistic in vitro.

Preemptive and prophylactic therapy with ganciclovir (GCV) appears to provide some clinical benefit to immunocompromised individuals infected with HCMV, however resistance to the drug occurs frequently in this population and appears to be related to levels of viral replication that occur notwithstanding GCV therapy (Gilbert et al. 2002). Although PFA and CDV are available to treat resistant infections, their associated renal toxicity limits their utility (Torres-Madriz and Boucher 2008).

Alkoxyalkyl and alkyl esters of esters of nucleoside analogs, such as hexadecyloxypropyl-CDV (CMX001) have also been shown to be exceedingly active against this virus and are effective against drug resistant isolates of the virus (Wan et al., 2005; Williams-Aziz et al. 2005). This compound exhibits excellent antiviral efficacy against HCMV that is a thousand fold greater than that of CDV against HCMV. It also has greatly improved the efficacy against other DNA viruses including adenovirus (Hartline et al. 2005). The broad spectrum of antiviral activity of this compound suggests that it might be useful in the therapy of transplant recipients, which are often infected with multiple viruses.

We evaluated the efficacy and cytotoxicity of this compound against HCMV in combination with GCV since it will likely be used in patients that are resistant to this drug.

Combination Studies with CMX001 and GCV

Combinations of CMX001 and GCV were evaluated using an in vitro antiviral assay to assess combined efficacy by methods similar to those described previously (Prichard and Shipman, 1990). Briefly, a checkerboard matrix of drug dilutions was prepared with BioMek 2000 directly in 96-well plates containing monolayers of human foreskin fibroblast (HFF) cells. Four replicate plates were infected at a multiplicity of infection (MOI) of 0.001 PFU/cell, incubated for 7 days, and viral load quantified by real time PCR. Two replicate plates remained uninfected, and cytotoxicity was evaluated with CellTiter-Gla at 7 days.

### Quantitative PCR Assays

Inhibition of viral DNA synthesis was determined in monolayers of HFF cells in 96-well plates infected with the AD169 strain of HCMV at an MOI of 0.01 PFU/cell. Compound dilutions were added to the infected monolayers, which were incubated for 7 days. DNA from assay plates was isolated with a 96-well Wizard kit (Promega), and quantified by real time PCR using primers 5′-AGG TCT TCA AGG AAC TCA GCA AGA-3′ and 5′-CGG CAA TCG GTT TGT TGT AAA-3′, and the probe 5′-(6FAM) CCG TCA GCC ATT CTC TCG GC TAMRA-3′. Absolute quantitation of viral copy number was performed using a standard curve with dilutions of a plasmid DNA (pMP217) containing sequences homologous to the amplified fragment.

### HCMV Real Time Array

A real time array was developed to provide a global analysis of HCMV gene expression. This technique quantifies mRNA levels from 139 viral genes by real time PCR. The analysis is performed on two assay plates containing primers for the viral genes as well as two cellular housekeeping genes that are used to normalize the experimental data and quantify viral transcripts by the ΔΔCt method.

Monolayers of primary lung fibroblast cells (HEL299, ATCC) were prepared in 6-well plates and incubated for 3 days prior to infection. Cell monolayers were then infected with the AD169 strain of HCMV (as the HB5 BAC strain) at an MOI of 1.0 PFU/cell. After a 1 h adsorption period, compounds are added to triplicate wells. Concentrations used in these studies are as follows: CDV 50 μM, GCV 15 μM, and CMX001 0.5 μM. Total RNA from triplicate wells was harvested and isolated with Rneasy columns (Qiagen). Residual DNA was degraded with RNAse-free DNAse. cDNA was prepared by MuLV reverse transcriptase and oligo dT, Viral mRNA from triplicate wells was quantified by real time PCR. Data were normalized to housekeeping genes and statistically significant changes were determined by ANOVA (P<0.05).

### Drug Combination Studies

Previous studies done in our laboratory demonstrated the efficacy of CMX001 against the herpesviruses and orthopoxviruses. The exceptional efficacy against HCMV, including isolates that are resistant to GCV suggested that the compound might be useful in the treatment of resistant infections. Combinations of CMX001 and GCV were evaluated to assess their combined efficacy against HCMV. The evaluation of combined cytotoxicity was performed concurrently and no synergistic cytotoxicity was observed (data not included). Both CMX001 and GCV were effective in reducing the accumulation of viral DNA (FIG. 22). CMX001 is a more potent inhibitor of viral replication as measured by real time PCR. Concentrations of CMX001 above 10 nM essentially eliminated the amplification of viral DNA, which remained at or below the level of input DNA.

The efficacy of CMX001 and GCV combinations were evaluated and analyzed by methods reported previously (Prichard and Shipman, 1990). The MacSynergy II software is available for free download at the following website: http://medicine.uab.edu/Peds/69011/. An analysis of the interactions showed that the combination synergistically inhibited the accumulation of viral DNA (FIG. 23). The volume of synergy was comparatively low (2.2 log₁₀ ge/ml), and was expected since both compounds inhibit the DNA polymerase.

Cytotoxicity was also evaluated concurrently using a CellTiter-Glo assays (Promega). Combinations of both agents were well tolerated and did not result in synergistic cytotoxicity (FIG. 24).

### CMX001 and GCV Reduce Expression of Most HCMV Open Reading Frames

The exceptional inhibition of viral DNA synthesis by CMX001 was investigated further by examining the transcriptional profile of viral genes in cultures treated with this compound (FIG. 25). Responses to compounds with different mechanisms of action result in distinct transcriptional profiles.

Statistically significant reductions in mRNA levels were identified by ANOVA (P<0.05). Significant reductions in levels of most viral transcripts were observed in response to GCV, CDV, and CMX001. This included both early and late viral genes and was unexpected. The general pattern of inhibition was similar for these three inhibitors of DNA synthesis and suggests that this pattern is a characteristic of drugs with this mechanism of action.

The potent inhibition of DNA accumulation by CMX001 did not appear to increase the number of viral transcripts affected, but resulted in greater reductions in the quantities of the transcripts. Reductions in viral transcripts in response to CDV and CMX correlated well (FIG. 26).

The transcriptional profiles induced by three DNA synthesis inhibitors were similar and appear to be a characteristic of this mechanism of action. Changes in Ct values associated with CDV and CMX001 (a prodrug of CDV) correlated well. These responses also correlated well with that observed for GCV and suggest that this transcriptional profile is associated with inhibitors of viral DNA synthesis.

CMX001 is a very potent inhibitor of HCMV replication and can reduce the accumulation of viral DNA synthesis by at least three orders of magnitude. Combinations of CMX001 and GCV synergistically inhibit viral replication and suggest that additional in vivo studies are warranted. No synergistic cytotoxicity was observed. Transcriptional changes induced by CMX001 are very similar to those induced by CDV and GCV which would be predicted for this inhibitor of viral DNA synthesis. The large decrease in genome copy number induced by CMX001 does appear to change the number of transcripts affected, but rather impacts the magnitude of their decreased accumulation.

### Example 15

### CMX001 and Acyclovir Combination Therapy for Treatment of Herpes Simplex Virus Infection

Previous studies have shown that either CMX001 or acyclovir (ACV) are effective in vitro against herpes simplex virus (HSV) isolates and in preventing mortality of mice infected intranasally with HSV-1 or 2. Evaluation of efficacy using suboptimal doses of these two agents in combination has not been reported previously. In cell culture, CMX001 was evaluated against a panel of both wild-type and ACV-resistant isolates of HSV-1 and HSV-2 and found to be highly effective with EC50 values ranging from 0.008 to 0.03 μM. These virus isolates were also inhibited by concentrations of ACV ranging from 2.0 to >100 μM. Using various concentrations of CMX001 and ACV in combination in tissue culture cells resulted in synergistic efficacy with no increase in toxicity. To determine if this combination would result in enhanced efficacy in an animal model, CMX001 was given once daily at 1.25, 0.42 or 0.125 mg/kg with or without ACV to mice infected intranasally with HSV-2. ACV was given twice daily at 30, 10 or 3 mg/kg. Treatments were initiated 72 hr post viral infection by oral gavage for 7 days. As expected from previous work where 5 mg/kg was an optimal dose of CMX001 in this model, CMX001 as a single therapy at 1.25, 0.42 or 0.125 mg/kg did not significantly improve survival or increase the mean day to death (MDD). ACV alone improved survival at 30 mg/kg (p=0.06) and significantly increased the mean day to death at 30 or 10 mg/kg (p<0.01), but not at 3 mg/kg. Suboptimal doses of CMX001 and ACV together, significantly enhanced protection from mortality or increased the MDD compared with either drug alone in 8 of 9 combination groups. No additive toxicity was detected. These results indicated that low dose combinations of these two agents act synergistically in vitro and in vivo and should be considered for use in herpesvirus infections in humans.

### Materials and Methods

In vitro screening by CPE assay in human foreskin fibroblast (HFF) cells with confirmation using plaque reduction assays in HFF.

Animals: BALB/c, female mice, 3-4 weeks of age.

Viral Inoculations: Intranasal, 0.04 ml using 1.1×10⁵ pfu/mouse, an approximate LID₉₀.

Virus Stocks: Herpes Simplex Viruses, type 1, strains E-377, F, HL-3, DM2.1, B-2006, PAAr5, and SC16-S1; or HSV, type 2, strains MS, G, SR, AG-3, 12247, 11680, or 11572.

Antiviral Compounds : CMX001 (hexadecyloxypropyl-CDV or HDP-CDV), cidofovir (CDV) or acyclovir (ACV).

Treatments were administered to mice for 7 consecutive days beginning 72 hr post viral inoculation by oral gavage using a 0.2 ml volume. CMX001 was administered once daily and ACV was given twice daily at approximately 12 hr intervals.

Mortality rates analyzed by Fisher's exact test; Mean day of death (MDD) by Mann-Whitney U rank sum.

For combination studies, a 24 hr incubation of single or combined antiviral drugs was performed using low passage HFF. CMX001 was added using concentrations from 0 to 500 nM with or without ACV using concentrations of 0 to 20 μM for determination of effects against HSV-2, MS replication by Real Time PCR. Statistical significance of 95% confidence levels were determined by the MacSynergy program.

### Results

In vitro efficacy of CMX001, CDV or ACV as single agents against wild type strains of HSV-1 or -2 are shown in Table 8. Efficacy of CMX001 and ACV against ACV-resistant strains of HSV-1 or -2 are shown in Table 9. The EC₅₀values indicate CMX001 is more effective in vitro than CDV. The combinations of CMX001 with ACV resulted in synergy without increases in toxicity as shown in FIG. 27.

When CMX001 was administered orally (p.o.) using suboptimal doses of 1.25, 0.42 or 0.125 mg/kg once daily for 7 days to mice infected with HSV-2, MS mortality was not significantly reduced when treatments were initiated 72 hr post viral inoculation nor was the mean day to death extended. When twice daily treatments of ACV using suboptimal doses of 30, 10 or 3 mg/kg were started 72 hr post viral inoculation, mortality was not significantly reduced, but mean day to death was significantly extended at the 30 and 10 mg/kg doses. Combinations of CMX001 with ACV, however, improved either the survival or time to death in the majority of groups when compared to single monotherapy (Table 10).

TABLE 8
In Vitro Efficacy of CMX001 Against Wild Type Strains of HSV-1 and HSV-2

| | HSV-1 | | | HSV-2 | | |
|---|---|---|---|---|---|---|
| Compound | E-377 | F | HL-3 | MS | G | SR |
| CMX001 | 0.06 ± 0.03^{a} | 0.01 ± 0.005 | 0.01 ± 0.01 | 0.08 ± 0.07 | 0.01 ± 0.01 | 0.02 ± 0.02 |
| CDV | 5.5 ± 6.0 | 1.5 ± 0.4 | 3.6 ± 3.0 | 5.1 ± 5.4 | 5.5 ± 2.1 | 5.9 ± 5.9 |
| ACV | 2.5 ± 1.3 | 0.35 ± 0.2 | 3.8 ± 0.4 | 2.2 ± 0.8 | 3.1 ± 3.5 | 4.4 ± 0.6 |

^{a}EC50 Values in μM

TABLE 9
In Vitro Efficacy of CMX001 or ACV Against ACV
Resistant Strains of HSV-1 and HSV-2

| Virus | Strain | CMX-001 | ACV |
|---|---|---|---|
| HSV-1 | DM2.1 | 0.008 ± 0.0007^{a} | >100 ± 0 |
| B-2006 | | 0.01 ± 0.0007 | >100 ± 0 |
| PAAr5 | | 0.01 ± 0 | 13.6 ± 2.7 |
| SC16-S1 | | 0.01 ± 0 | >100 ± 0 |
| F (wild type) | | 0.015 ± 0.006 | 1.9 ± 0.47 |
| HSV-2 | AG-3 | 0.023 ± 0.009 | >100 ± 0 |
| 12247 | | 0.021 ± 0.012 | >100 ± 0 |
| 11680 | | 0.009 ± 0.0007 | 79 ± 10 |
| 11572 | | 0.027 ± 0.018 | >100 ± 0 |
| G (wild type) | | 0.029 ± 0.015 | 2.1 ± 0.99 |

^{a}Values shown as EC₅₀ μM

TABLE 10
Effect of Treatment with CMX001 in Combination with ACV on Mortality of BALB/c Mice
Infected with HSV-2, strain MS

| | Mortality | | | | |
|---|---|---|---|---|---|
| Treatment + 72 hr | Number | Percent | P-value | MDD^{a} | P-value |
| Untreated | 12/15 | 80 | — | 8.5. ± 0.9 | — |
| Vehicle-0.4% CMC | 12/15 | 80 | — | 8.0 ± 1.4 | — |
| Acyclovir 30 mg/kg | 6/15 | 40 | 0.06 | 10.7 ± 2.0 | 0.01 |
| 10 mg/kg | 11/15 | 79 | NS^{a} | 11.1 ± 2.4 | 0.01 |
| 3 mg/kg | 15/15 | 100 | NS | 9.0 ± 1.6 | NS |
| CMX001 1.25 mg/kg | 9/15 | 60 | NS | 9.4 ± 2.9 | NS |
| 0.42 mg/kg | 12/15 | 80 | NS | 9.8 ± 2.6 | NS |
| 0.125 mg/kg | 13/15 | 87 | NS | 9.0 ± 1.0 | 0.06 |
| ACV 30 + CMX 1.25 | 6/15 | 40 | 0.06 | 11.0 ± | NS |
| ACV 30 + CMX 0.42 | 4/15 | 27 | <0.01 | 4.7 | <0.01 |
| ACV 30 + CMX 0.125 | 2/15 | 13 | 0.001 | 12.8 ± | <0.05 |
| ACV 10 + CMX 1.25 | 3/15 | 20 | <0.01 | 4.1 | 0.05 |
| ACV 10 + CMX 0.42 | 8/15 | 52 | NS | 10.5 ± | 0.06 |
| ACV 10 + CMX 0.125 | 6/15 | 40 | 0.06 | 0.7 | 0.05 |
| ACV 3 + CMX 1.25 | 6/15 | 40 | 0.06 | 9.7 ± 0.6 | <0.01 |
| ACV 3 + CMX 0.42 | 8/15 | 53 | NS | 10.4 ± | <0.01 |
| ACV 3 + CMX 0.125 | 10/15 | 67 | NS | 3.4 | NS |
| | | | | 9.8 ± 1.7 | |
| | | | | 11.3 ± 2.5 | |
| | | | | 10.3 ± 1.9 | |
| | | | | 9.2 ± 2.2 | |

In vitro efficacy of combinations of CMX001 with ACV indicated that synergistic activity against HSV-2, strain MS occurred using nanomolar concentrations of CMX001 with 20 or less μM concentrations of ACV. The use of suboptimal doses of CMX001 or ACV in vivo as single agents caused an increase in the MDD for the higher doses of ACV, but did not reduce mortalities using CMX001 or ACV when treatments were initiated 72 hrs post viral inoculation in mice. In mice infected with HSV-2, combination therapy significantly increased survival or mean day to death when compared to vehicle treated groups or monotherapy treated groups.

### Example 16

### CMX001 and Polyomavirus BK

Effective antiviral drugs for treating polyomavirus BK (BKV) replication in polyomavirus-associated nephropathy or -hemorrhagic cystitis are of considerable clinical interest. Unlike cidofovir, the lipid conjugate 1-O-hexadecyloxypropyl-cidofovir (CMX001) is orally available and has limited nephrotoxicity in rodent models and human studies. Primary human renal proximal tubular epithelial cells were infected with BKV-Dunlop and CMX001 was added 2 h post-infection (hpi). Intracellular and extracellular BKV DNA load was determined by quantitative PCR, viral early and late gene expression by reverse transcription PCR, western blotting, and immunofluorescence microscopy. The host cell was also examined regarding viability, metabolic activity, DNA replication and real-time proliferation. Titration of CMX001 identified 0.31 μM as the inhibitory concentration reducing the extracellular BKV load at 72 hpi by 90% (IC-90). We found no effect on BKV large T-antigen mRNA and protein expression at 24 hpi, but subsequent BKV genome replication as measured by intracellular loads was reduced by 90% at 48 hpi. Late gene expression was reduced by 70-90% at 48 and 72 hpi. Comparing exposure from 24, 48, 72 and 96 h on BKV loads at 96 hpi, it was found that CMX001 IC-90 inhibition was rapid and more enduring than cidofovir IC-90. CMX001 0.31 μM had little effect on overall cell metabolism, but reduced BrdU incorporation and host cell proliferation by 20-30%, while BKV infection increased cell proliferation rate of both, exponential and near-confluent cultures. It was concluded that CMX001 inhibits BKV replication with a longer lasting effect than cidofovir at 400× lower levels, with lesser side effects on relevant host cells in vitro.

In vitro studies have shown effect of CDV on BKV replication in human embryonic lung fibroblast cells (WI-38) and in primary human renal tubular epithelial cells (RPTECs). In RPTECs, CDV inhibited BKV replication in a dose-dependent manner with a 90% inhibitory concentration (IC-90) at 40 ug/mL. The inhibition was mediated at the step of BKV DNA replication, but also decreased both host cellular DNA replication and metabolic activity as correlates of nephrotoxicity in vivo. Another caveat of CDV is that it must be given intravenous and the patients therefore need to be hospitalized. Recently a 1-O-hexadecyl-oxypropyl lipid conjugate of CDV (HDP-CDV) denoted CMX001 was developed. Unlike CDV, the conjugate seems to be taken up by cells similar to lysophosphatidylcholine where CDV as active compound is liberated by phospholipase cleavage. Studies of single and repeated dosing in animals and in human volunteers ranging from 0.1 mg/kg to 4.0 mg/kg showed no evidence of nephrotoxicity. In a previous study, CMX001 has been reported to inhibit BKV replication in human fetal fibroblasts, but the mechanistic details were not reported. The effects of CMX001 on BKV replication in RPTECs are reported herein, which is the primary target of BKV in PyVAN.

### Materials and Methods

### Cells and Virus

Primary human renal proximal tubule epithelial cells (RPTECs) (Lonza, www.lonzabioscience.com) were propagated as described by the manufacturer. All experiments were performed with RPTECs passage 4 and BKV-Dunlop supernatants or gradient-purified virus from Vero cells.

### Infection and CMX001 Treatment

Before each experiment, CMX001 was freshly dissolved to 1 mg/ml in methanol/water/ammonium hydroxide (50/50/2) and further diluted in RPTEC growth medium. About 50% confluent RPTECs were infected with BKV (Dunlop) MOI 1. After 2 h incubation at 37° C., the virus was replaced with fresh medium with or without CMX001 unless otherwise stated,

### Cytotoxicity and Cell Proliferation Assay

The mitocondrial metabolic activity was monitored by the colorimetric WST-1 assay (Roche, Rotkreuz, Switzerland) measuring reduction of the Tetrazolium salt, WST-1, by mitochondrial dehydrogenases. DNA synthesis was quantified by colorimetric measurement of BrdU incorporation into DNA using the “Cell proliferation ELISA, BrdU” kit (Roche). The attachment and proliferation of the cells was measured as impedance using E-plates and the xCelligenee system (Roche). In order for the RPTECs to attach and proliferate on the E-plates, the plates were first coated with fibronectin. Next the background impedance of the plates was monitored by addition of 100 medium to each well, before the plate was connected to the system and checked in the cell culture incubator for proper electrical-contacts. Subsequently, 100 μl cell suspension containing the indicated cell numbers was seeded. To determine the effect of BKV infection and CMX001 treatment, about 24 h after seeding 150 μl of the media was replaced with fresh media with or without purified BKV-Dunlop in the presence or absence of CMX001 (final concentration of 0.31 nM). The cells were grown for 96 h and impedance was measured every 15 minutes for the first 6 h then every 30 minutes. Impedance was expressed as an arbitrary unit called the Cell Index.

RNA Extraction and cDNA Synthesis

At 24, 48 and 72 hpi cells were lysed and total RNA extracted using the mirVana PARIS kit (Ambion). RNA samples were treated with DNase turbo (Ambion) to remove residual DNA before the RNA quality was checked by agarose gel electrophoresis and RNA concentration was determined. cDNA was generated from 250 ng RNA per sample using the High Capacity cDNA kit (Applied Biosystems).

### DNA Extraction

To assay extracellular BKV loads, cell culture supernatants were harvested at 24, 48 and 72 hpi and frozen in −70° C. until automatic extraction by a robot (GenoM-48, Qiagen, www.qiagen.com). For intracellular BKV loads, cells were washed, trypsinized and pelleted at 220 g for 10 min, resuspended in G2 buffer from the MagAttract DNA Mini M48 kit (Qiagen) and frozen at −70° C. until extraction with the same robot.

### Quantitative PCR for BKV DNA and Cellular Gene Detection

To quantify intracellular or extracellular BKV DNA load, a quantitative PCR (qPCR) with primers and probe targeting the large T-antigen (LT-ag) gene was used. For normalization of intracellular BKV DNA each sample was analyzed in parallel by the qPCR for the gene for aspartoacylase (ACY) to correct for cellular DNA.

### Western Blotting

Cells were lysed in Cell Disruption buffer (mirVana PARIS kit, Ambion) 24, 48 and 72 hpi and stored at −70° C. until separation with SDS-polyacrylamide gel electrophoresis (SDS-PAGE) followed by blotting onto PVDF membrane. Detection of BKV and cellular proteins was performed with polyclonal rabbit antiserum directed against LT-ag (1:2000), VP1 (1:10000), or agno (1:10000) (1, 27) and a monoclonal mouse antibody directed against GAPDH (Ab8245; 1:5000, Abeam, www.abcam.com) followed by anti-rabbit and anti-mouse infrared dye-labeled secondary antibodies (IR Dye 800, Rockland, www.rockland-inc.com and Alexa Fluor 680, Invitrogen, www.invitrogen.com) both 1:5000 before detection with Licor Odyssey Infrared detection system.

### Immunofluorescence Staining and Digital Image Processing

Cells were washed in PBS, methanol fixed, blocked with 3% goat-serum in PBS for 30 min, followed by subsequently incubation with primary (37° C.) and secondary (r.t.) antibodies for 30 min. Primary antibodies were monoclonal anti-SV40 LT-ag antibody (Ab-2 Pab416; 1:100, Chemicon, www.ehemicon.com) and polyclonal rabbit antiserum directed against agno or VP1 (both 1:1000). The secondary antibodies were anti-mouse conjugated with AlexaFluor 568 and anti-rabbit conjugated with AlexaFluor 488 (1:500; Molecular Probes, www.invitrogen.com). Nuclei were labeled with DRAQ5™ (Biostatus, www.biostatus.com). Images were collected using a Nikon TE2000 microscope equipped and processed with NIS Elements Basic Research software version 2.2 (Nikon Corporation).

### Results

### Determination of Inhibitory Concentration IC-90

To investigate the effect of CMX001 on BKV progeny, increasing concentrations of CMX001 were added 2 hpi and supernatants harvested at 72 hpi. We observed that CMX001 reduced the extracellular BKV load in a concentration dependent manner (FIG. 28a). When viral input was subtracted, CMX001 0.31 μM reduced the BKV load on average by 90% defining the inhibitory concentration IC-90. In agreement with this immunofluorescence staining of BKV-infected RPTECs 72 hpi demonstrated a concentration dependent decrease in number and intensity of large T-antigen (LT-ag) and agno expressing cells (FIG. 28b). At concentrations as high as 10 μM CMX001, no BKV-infected cell was observed, but the total cell number was significantly reduced (data not shown; see also below). It was concluded that CMX001 reduced the expression of early and late BKV proteins and the production of extracellular progeny, but also seemed to have a concentration-dependent effect on the proliferation of RPTECs.

### CMX001 and BKV Early Gene Expression

To study the effect of CMX001 IC-90 on BKV early gene expression, the LT-ag mRNA levels in treated and untreated RPTECS at 24, 48 and 72 hpi by quantitative reverse transcription PCR (qRT-PCR) were compared. The results were normalized to the housekeeping gene huHPRT and presented as the changes relative to the untreated sample at 24 hpi. No difference was found in early gene expression at 24 hpi, but a reduction of 33% and 64% was seen at 48 hpi and 72 hpi, respectively (FIG. 29a). Analyzing LT-ag expression by western blotting revealed a corresponding result showing little difference at 24 hpi, but a 20% to 30% reduction at the later time points (FIG. 29b). It was concluded that CMX001 did not inhibit BKV early protein expression early in the viral life cycle, but later at 48 and 72 hpi.

### CMX001 and BKV Genome Replication

Since BKV episome replication is known to occur around 36 hpi, it was investigated whether the BKV genome replication was affected by CMX001. Intracellular BKV load at 24, 48 and 72 hpi was measured by qPCR and normalized to the cell number using the aspartoacylase (ACY) as a cellular reference gene. Compared to untreated RPTECs, CMX001 at 0.31 μM reduced the intracellular BKV load by 94% at 48 h and 91% at 72 hpi (FIG. 29c). Thus, a significant inhibitory effect of CMX001 on intracellular BKV genome replication was identified. This step is known to require LT-ag function which increases viral late gene expression by two synergistic mechanisms, namely by increasing the DNA templates thereby the gene dosis per per cell for late gene transcription and by activating transcription from the late promoter.

### CMX001 and BKV Late Gene Expression

To study the effect of CMX001 on BKV late gene expression, the VP1 and agno mRNA levels in CMX001 treated and untreated RPTECS at 24, 48 and 72 hpi by RT-qPCR were compared. Late mRNA levels were normalized to the housekeeping gene huHPRT and presented as the changes relative to the untreated sample at 24 hpi. A 93% and 82% reduction was found at 48 and 72 hpi, respectively (FIG. 30a). By western blotting, a decrease of VP1 of 85 and 96%, respectively, was found while agno was reduced by 97 and 96% (FIG. 30b).

To investigate the effects of CMX001 on BKV gene expression at the single-cell level, immunofluorescence for the early LT-ag and the late agno at 48 hpi and 72 hpi was performed. It was found that at 48 hpi, the number and the intensity of nuclear LT-ag signals was slightly reduced. The decrease was more pronounced for the late agno expression. At 72 hpi, LT-ag and agno expression had increased in the CMX001 treated wells, but to a lesser extent than in the untreated wells (FIG. 30c). Interestingly, immunofluorescence also revealed some refractory cells in the CMX001 treated culture expressing agno at levels comparable to untreated cells even with CMX001 concentrations up to 2.5 μM (FIG. 28b). It was concluded that CMX001 significantly reduces late protein expression but also inhibit early protein expression at later time points after BKV genome replication had occurred.

### Kinetics of CMX001 Inhibition

To examine the kinetics of CMX001 inhibition on BKV replication, RPTECs were treated after infection for 24 h, 48 h, 72 h or 96 h and BKV loads were determined in the supernatants at 96 hpi. At the indicated times, the supernatant was harvested, the cells were washed once and new complete medium was added. At 96 hpi, BKV loads were measured in the supernatants. As shown, CMX001 treatment at 0.31 μM for 24 h was enough to reduce the BKV load at 96 hpi by approximately 90% (FIG. 31a). Longer exposure times had only a marginal effect. Under these conditions, CDV treatment at 40 μg/mL for at least 48 h was needed to reduce the extracellular BKV load to this level. To investigate whether or not the observed reductions in BKV load corresponded to a reduced number of infectious units, the supernatant harvested at the different timepoints were 10-fold diluted and seeded on new RPTECs. Three days p.i. cells were fixed and immunofluoresence staining with antibodies against LT-ag and agno performed. The results demonstrated that little infectious virus was detectable after treating cells with CMX001 for only 24 h, while CDV treatment for 72 h was needed to obtain a similar result (FIG. 31b). It was concluded that CMX001 has a more rapid and enduring inhibitory effect than CDV.

### Effects of CMX001 on RPTECs

Phase contrast microscopy did not reveal any crude signs of impaired host cell viability during the 3 day exposure to CMX001 at 0.31 μM. To use more sensitive assays, the host cell DNA replication and metabolic activity were investigated using BrdU incorporation and WST-1 assays in uninfected and infected RPTECs. It was found that CMX001 reduced both DNA replication and metabolic activity of infected RPTECs in a concentration-dependent manner (FIG. 32a). Of note, CMX001 at 0.31 μM, the IC-90 of BKV replication, induced a 25% reduction in BrdU incorporation, but no significantly altered metabolic activity.

To investigate the influence of CMX001 at 0.31 μM on proliferation of uninfected and BKV infected RPTECs in real time, the impedance in arbitrary cell index units was measured using the xCelligence system. Cells were at two different densities one that permitted exponential growth up to 72 h (2000 cells/well, bottom), and one at subconfluency entering confluency within the first 24 h after seeding (12000 cells/well, top). One day post-seeding, the medium was replaced, and four conditions examined: i) uninfected and untreated, ii) uninfected, but CMX001 treated, iii) BKV infected, but untreated or iv) BKV infected and CMX001 treated. The cell index was measured in 30 min intervals up to 96 h. The data showed that BKV infection increased cell proliferation in exponentially growing and in subcontinent cell cultures (FIG. 32b). In exponentially growing cells, CMX001 reduced the rate of RPTEC proliferation by approximately 25% in uninfected cells and by approximately 35% in BKV infected cells at 48 h postexposure (72 h after seeding). In subcontinent cells, CMX001 had only a minimal inhibitory effect on infected and uninfected cells alike. It was concluded that CMX001 at IC-90 of 0.31 μM had a certain inhibitory effect on RPTEC proliferation which was inversely proportional to cell density, but did not appear to be toxic at this concentration.

### Discussion

Antiviral drugs with higher efficacy and specificity are needed to improve current outcomes of BKV-mediated polyomavirus-associated nephropathy after kidney transplantation and hemorrhagic cystitis after allogenic hematopoietic stem cell transplantation. In this study, CMX001 was characterized with respect to its inhibitory activity regarding BKV replication in human primary proximal tubular epithelial cells. The results demonstrate that CMX001 at 0.31 μM was sufficient to reduce the extracellular progeny BKV load by 90% at 72 hpi. Investigation of the BKV life cycle indicated that CMX001 inhibition occurred after the initial early gene expression at 24 hpi at the level of BKV genome replication. Compared to untreated controls, the intracellular BKV genome loads were not increasing between 24 and 48 hpi. Moreover, the subsequent burst of late gene expression at 48 and 72 hpi was significantly reduced which in untreated cells results from the synergy of LT-ag mediated activation and the higher gene copy number of DNA templates. CMX001 was active at about 400 times lower concentrations than the IC-90 for CDV in the same test system (CDV IC-90 40 ug/ml=127 uM versus CMX001 IC-90 0.31uM). The inhibitory activity of the CMX001 was more immediate and enduring compared to the CDV requiring an exposure time of 24 h as compared to 48 h to 72 h for CDV for an IC-90 of BKV progeny loads at 96 hpi. This difference in inhibitory kinetics was also apparent in infectious units when seeding diluted supernatants onto new RPTECs and seems to be result from lysophosphatidylic-like modification with the improved uptake and high intracellular concentrations. Taken together, the data indicate a significantly enhanced BKV-inhibitory potency of the lysophosphatidylic-like derivative CMX001 over the parent compound CDV.

Previous work on CDV indicated that the inhibitory activity of CDV was closely linked to inhibitory effects of the host cells: CDV IC-90 reduced the proliferation of RPTEC by 30%-40% according to BrdU incorporation, while the overall metabolic activity was reduced by 20% to 30% (1). Given the increased potency of CMX001 on BKV replication, it was of considerable interest to monitor effects on the host cells. Results indicated that CMX001 IC-90 had only little effect on the overall metabolic activity of BKV-infected RPTECs and reduced the overall proliferative activity by up to 25%. As reported previously, BKV infection by itself increased the metabolic activity of RPTECs over uninfected cells and increased the proliferative activity as measured by BrdU incorporation. Comparing RPTEC proliferation in a novel real-time proliferation assay, the stimulating effect of BKV infection on cell proliferation was clearly demonstrated on exponentially growing as well as on cell cultures reaching confluency. In accordance with the BrdU results, this assay showed that CMX001 BKV IC-90 reduced the proliferation rate of exponentially growing RPTECs by approximately 25%. This effect was less apparent at higher cell densities suggesting that the specificity of CMX001 on BKV infection increased when confluent cells are infected. It can be envisaged that this feature might also contribute to an overall reduced nephrotoxicity especially when treating focal diseases such as polyomavirus-associated nephropathy. Since CMX001 pretreatment for 24 h was also effective to reduce BKV progeny loads, this might contribute to an effective antiviral state without excessive toxicity and contribute to a significant clearance rate by further lowering the R₀ to below 1.

CMX001 was found to inhibit BKV replication in human embryonic lung fibroblasts cells (WI-38) with a more than 800-fold increased effective concentration (EC)-50 of 0.13 μM compared to the 115.1 μM observed for CDV. These results were obtained by determining the BKV loads of cells harvested 7 days after infection and normalising to the host cell load using a house keeping gene for the cytotoxic concentration (CC)-50 and indicated a selectivity index (SI)-50 of 113. Results aimed at determining the IC-90 in RPTECs needed to clear viremia and viruria by 3 and 10 weeks, respectively, according to a detailed infection model of polyomavirus-associated nephropathy in kidney transplants. Since BrdU incorporation at CMX001 concentration of 10 μM was reduced to approximately 10% of untreated infected cells (CC-90), one could estimate the SI-90 as being 62.5. This SI must be regarded as very favourable for pre-clinical and clinical study and extend earlier observations to a clinical relevant host cell model of primary human tubular epithelial cells.

Taken together, it is concluded that CMX001 like CDV inhibits BKV replication in primary human RPTECs downstream of initial LT-ag expression at the level of viral genome replication. Although polyomavirus replication is dependent on host cell DNA polymerase function, the improved BKV specificity may result from activation of infected cells and the preferential recruitment of replication to the site of viral genome replication mediated by LT-ag. The lysophatidylic modification causes a more rapid and enduring antiviral effect of CMX001 at approximately 400-fold times lower concentration than for CDV and an estimated SI-90 of 62.5.

### Example 17

### CMX001 and JC Virus

The effect of CMX001 in comparison to CDV on replication of JCV in the human fetal glial cell line SVG was investigated. Limited cytotoxicity for CMX001 in SVG cells was observed for concentrations between 0.01 to 0.1 μM. CMX001 caused a dose dependent decrease of JCV-infected cells during initial infection and virtually eliminated of JCV-infected cells during a previously established infection, which appeared to be due to a defect at the level of viral DNA replication. Suppression of JCV infection at concentrations that are not toxic to the human glial cells and increased bioavailability suggests a potential use of CMX001 to limit JCV multiplication in PML patients.

### Materials and Methods

### Cells and Virus

SVG cells were generated by transfecting human fetal glial cultures with an origin-defective SV40 mutant and growing the resultant culture of cells that are immortalized by stable expression of SV40 T antigen. SVG cells were maintained in minimal essential medium (MEM) supplemented with 10% FBS, 2 mM L-glutamine, and penicillin/streptomycin. The Mad-4 variant of JCV was grown in and purified from human fetal brain progenitor derived astrocytes. Virus concentration was determined by hemagglutination (HA) of human type O erythrocytes.

### JC Virus Infection.

SVG cells were seeded at densities of 1×10⁴-2×10⁴ cells per well in 96-well plates or 3×0⁵ cells per well in 6-well plates. Cells were grown overnight at 37° C. The culture medium was then removed and cells were washed 3 times with phosphate buffered saline (PBS). Cells were exposed to a minimal volume of PBS containing Mad-4 JCV at a concentration of 10 hemagglutinin units (HAU) per 5×10⁴ cells for 90 minutes. Culture medium was added to each well to the nominal volume of the culture plate. The non-infected control cultures incubated with PBS for 90 minutes in the absence of virus. After overnight exposure to JCV the culture medium was replaced with drug containing media.

### Maintenance of JCV-Infected SVG Cultures.

Infected cultures of SVG cells were generated by exposing SVG cells to Mad-4 JCV at a concentration of 10 hemagglutinin units (HAU) per 5×10⁴ cells for 90 minutes. Culture medium was added to the nominal volume of the culture plate and cells were fed with new medium and carried for 7 to 11 passages. After 7 passages the culture was considered to be an established infection and was used in drug treatment experiments. Maintenance of JCV infection during cell passages was determined by in situ DNA hybridization to a JCV DNA specific probe.

### Drug Preparation and Dilutions.

The chemical structure, biological properties, and mechanism of action for the drugs used in this study are listed in Table 11. Cytosine β-D-arabinofuranoside (Ara-C) was obtained from Sigma-Aldrich (St. Louis, Mo.) and was stored as a 5 mg per mL stock in PBS at −20° C. Ara-C was diluted directly into cell culture medium at 5 and 20 μg per mL concentrations. Cidofovir (CDV) was obtained from Gilead (Foster City, Calif.) and was stored as a 1.2 M stock as an aqueous solution at room temperature. CDV was diluted directly into cell medium at 0.01, 0.03, 0.07, 0.1 and 1 μM concentrations. Hexadecyloxypropyl-cidofovir, CMX001, was obtained from Chimerix Inc (Durham, N.C.) and was stored as a 1.8 mM stock in methanol/water/ammonium hydroxide (50 vol: 50 vol: 2 vol) at 4° C. CMX001 was diluted directly into cell culture medium at concentrations of 0.01, 0.03, 0.07, 0.1 and 1 μM.

### In Situ DNA Hybridization.

Replication of viral DNA in JCV infected SVG cells was detected by in situ DNA hybridization using a full-length JCV biotinylated DNA BioProbe (Enzo Life Sciences, Inc., New York, N.Y.) as previously described in Houff, S. A., D. Katz, C. V. Kufta, and E. O. Major. (1989) “A rapid method for in situ hybridization for viral DNA in brain biopsies from patients with AIDS” AIDS 3:843-5. Calf thymus DNA was used as a non-specific control for the JCV probe.

### MTS Assay.

Quantification of cell viability using the MTS [3-(4,5-dimethylthiazol-2-yl)-5-(3-carboxymethoxyphenyl)-2-(4-sulfophenyl)-2H-tetrazoliuml assay is based on bioactivity of mitochondria dehydrogenase in living cells, which converts colorless tetrazolium salt to a colored formazan. MTS assays were performed according to manufacturer's instructions (Promega, Madison Wis.). Briefly, culture medium was removed from cells and 50 μL of PBS was added into each well of a 96 well plate. Ten μL of MTS reagent were added to wells of cells, and to a well without cells to determine background. Mixtures were incubated at 37° C. for 2 hours and the absorbance was measured at 490 nm. Trypan blue staining was performed on identical cultures to correlate MTS values with cell viability. The final values indicated are the result from 3 replicates. The value for the non-treated control was set to 100% and all other values are represented as a percentage of the control. Alamar blue (AB) assay.

Quantification of cell viability using the alamar blue (AB) assay was performed according to the manufacturer's instructions (Invitrogen, Gaithersburg, Md.). Briefly, 10 μL of AB reagent was added to each well of a 96 well dish containing cells or without cells to determine background. Mixtures were incubated at 37° C. for 6 hours and the absorbance measured at 570 nm using 600 nm as a reference wavelength. The value for the non-treated control was set to 100% and all other values are represented as a percentage of the control.

### Semi-Quantification of Hematoxyl in Intensity.

Because cell density in cultures processed for in situ DNA hybridization could not be measured by MTS or AB assays, cell density was approximated in cell cultures processed by quantifying hematoxylin staining intensity. Coverslips containing cells were prepared for in situ DNA hybridization and co-stained with hematoxylin. Subsequently, each coverslip was scanned and the hematoxylin intensities quantified using Image. To determine the total cell number per coverslip, images were acquired at 10 random positions at 100× magnification. Cells were counted throughout each image and the average cell number per slide was generated by averaging counts from the 10 images for each experimental group. Exactly 900 images at 100× magnification cover the surface of an 18 mm×18 mm coverslip.

### DNA Extraction.

DNA was harvested from non-infected and JCV-infected SVG cell cultures using the DNeasy® Blood & Tissue Kit according to manufacturer's instructions (Qiagen, Valencia, Calif.). DNA was quantified using a ND-8000 NanoDrop (Thermo Scientific, Wilmington, Del.) and was stored at 4° C. before use.

Quantitative Real-Time PCR (qPCR).

Quantification of JCV viral genome copy number in JCV infected SVG cultures was performed using a quantitative real-time PCR assay using a pair of JCV Mad-1 specific primers and probe targeting the nucleotide sequences of the N terminus of the viral T antigen as previously described in Ryschkewitsch, C., P. Jensen, J. Hou, G. Fahle, S. Fischer, and E. O. Major. (2004) “Comparison of PCR-southern hybridization and quantitative real-time PCR for the detection of JC and BK viral nucleotide sequences in urine and cerebrospinal fluid” J Viral Methods 121:217-21. Dual negative controls consisting of no template and DNA elution buffer were included to determine false-positives during each step of the purification process. A standard curve was generated using serial dilutions of the JCV Mad-1 plasmid, pM1_{TC}, ranging from 100 pg to 10 ag (attograms) and was used to calculate viral genome copy number for the infected cell cultures.

### Statistics.

The data were expressed as percentage of mean plus or minus the standard deviation (SD). Data were statistically evaluated at a significance level of 1% with One- or Two-Way ANOVA by using software VASSARSTATS followed by the Tukey HSD test.

### Results

### SVG Cells as a Tissue Culture Model to Measure the Activity of Drugs on JCV Infection.

Because JCV lytically replicates in glial cells of the central nervous system (CNS), SVG cells have been used for studies of the effects of drug treatment on JCV replication. SVG cells are a heterogeneous culture that resulted from immortalization of primary human fetal brain cultures with an origin-defective mutant of SV40. They are immortalized by stable expression of SV40 T antigen. SVG cells maintain the morphology of astrocytes with large flat cell bodies that are irregular in shape and contain a large nucleus (FIGS. 33a and 33b). An MIS assay was used to determine the growth kinetics and cell viability of SVG cultures. Cells were seeded at a range of densities and 24 hours post plating cell viability was determined by MTS assay and trypan blue staining. A standard curve for cell viability was generated by plotting MTS values (optimal densities) versus viable cell counts as determined by trypan blue staining as shown in FIG. 33c. As was expected, there was a linear relationship between MTS values and cell viability as observed by regression analysis (y=1×10″⁵×+0.0015, R²=0.9614). This standard curve was used to determine cell number based on MIS values in subsequent figures. To determine viability of SVG cultures over time, non-infected or JCV infected SVG cells were seeded into 96-well plates at 2×10³ per well, and MTS values were monitored at days 1, 2, 3, 4, and 7 post plating. The MTS values for each day (data not shown) were converted to cell number using the equation generated in FIG. 33c and were plotted versus time in days where day 0 is the time of plating. FIG. 33d demonstrates that cell number at day 1 was similar to the seeding amount of 2×10³ per well, indicating a lag period of 1 day for cell growth after cell plating. Cell grew exponentially during day 1 through day 3. By day 4, the non-infected culture contained more cells than the JCV-infected culture; however by day 7 post plating the cell numbers were similar in both cultures as they became confluent. No major difference in growth kinetics was observed between non-infected and JCV-infected SVG cells at the times tested since JCV replication requires between 7 to 14 days.

Because a previous study examining the effect of Ara-C on JCV replication showed that Ara-C is capable of reducing JCV infection in SVG cells, we sought to reproduce these results in the SVG cultures used in this study. SVG cells were exposed to Mad-4 JCV at a concentration of 10 HA units (HAU) per 5×10⁴ cells or PBS alone for the non-infected control. Approximately 24 hours after JCV exposure, non-infected and JCV-infected cells were treated with 0, 5, and 20 μg per mL of Ara-C. The culture media was replaced with new Ara-C containing media on day 4. Day 7 after JCV exposure, cell viability was measured by MIS analysis and active viral infection was measured by in situ DNA hybridization. JCV-positive cells, stained brown, were present in the JCV-infected culture and not in non-infected culture as shown in FIG. 34a. Duplicate plates of non-infected and JCV-infected SVG cells were also hybridized with a non-specific DNA probe; the non-specific probe was negative providing evidence for specificity of the JCV probe (data not shown). JCV-positive cells were quantified and the percentage of JCV positive cells was expressed as a percentage of the no Ara-C control (FIG. 34b). Ara-C treatment caused a statistically significant, dose dependant decrease in the percentage of cells containing JCV DNA of 25% for 5 μg per mL and 83% for 20 μg per mL (ρ<0.05). The effect of Ara-C on the cell viability was also determined (FIG. 34c). MTS analysis determined that 5 μg/ml Ara-C treatment did not cause cytotoxicity, but 20 μg/ml decreased cell viability by 21% (ρ<0.01). To determine if the reduction in the percentage of JCV DNA positive cells in the 20 μg per mL Ara-C treatment was due to an inhibitory effect of Ara-C on JCV rather than an indirect effect on reducing total cell number through cytotoxic affects, the percentages of cells containing JCV DNA were normalized to the number of viable cells present in the culture using the MTS reading (FIG. 34d). Normalization to cell viability showed that Ara-C treatment of 5 and 20 μg per mL reduced the percentage of JCV DNA containing cells by 27% and 78% respectively (ρ<0.05), which is similar to the values shown in FIG. 34b. These results demonstrate that Ara-C inhibits JCV replication in SVG cells. Therefore, SVG cells are a valid model in the present study to examine the effects of drug treatment on JCV replication in vitro.

### Limited Cytotoxicity of CMX001 to SVG Cells.

Treatment of PML with CDV has been associated with a positive prognosis in rare cases, however is not considered an efficacious treatment due to the inability to pass the BBB and high cytotoxicity. CMX001 is modified derivative of cidofovir that has demonstrated a higher level of potency than CDV for suppression of many viruses. To determine the effect of CMX001 on cell viability, SVG cells were treated with CMX001 or CDV at concentrations ranging from 0.01 to 1 μM for 4 days. CDV did not elicit any visible changes in cell density as detected by microscopy (FIG. 35a) or viability as measured by alamar blue (AB) staining (FIG. 35b) at a concentration range of 0.1 to 1 μM. Cell viability was quantified with AB staining because it is a more sensitive analysis than MTS. Importantly, cytotoxicity has been associated with higher concentrations of CDV of 20 to 50 μg per mL (63 and 159 μM). Treatment with CMX001 caused visible reduction in cell density and some morphological changes at 0.1 and 1 μM concentrations (FIG. 35a). In addition, treatment with CMX001 at all concentrations tested caused a reduction in cell viability including 6% at 0.01 μM, 11% at 0.1 μM, and 32% at 1 μM (ρ<0.01) as shown in FIG. 35b. From these results, we conclude that a concentration range between 0.01 and 0.1 μM is suitable for comparison of CMX001 and CDV for their effects on JCV due to limited affects on cell viability.

### CMX001 Suppresses JCV Replication in SVG Cells.

Confluent cultures of SVG cells were exposed to 10 hemagglutinin units (HAU) per 5×10⁴ cells of Mad-4 JCV in a 6 well plate. After overnight JCV exposure cells were treated with CMX001 or CDV at concentrations of 0.01, 0.03, 0.07 and 0.1 μM or drug diluent as a non-treated control for 4 days. JCV DNA replication was measured in the cultures by in situ DNA hybridization (FIG. 36a). JCV-positive cells were quantified and the percentage of JCV positive cells was expressed as a percentage of the non-treated control (FIG. 36b). CMX001 caused a dose-dependent reduction in the percentage of JCV DNA containing cells including 46%, 57% and 71% for the concentrations of 0.03, 0.07 and 0.1 μM, respectively. In contrast, CDV at same concentrations did not elicit any significant reduction in JCV DNA containing cells. Because CMX001 does affect cell viability at the concentrations tested, total cell number was determined from the coverslips used for quantification by in situ DNA hybridization. As illustrated in FIG. 36a, the density of cells did not change in the CDV-treated samples. However, CMX001 treatment resulted in a dose dependent reduction in cell density (FIG. 36c). To determine if the reduction in the percentage of JCV DNA positive cells upon CMX001 treatment was due to an inhibitory effect of the drug on JCV rather than an indirect effect on reducing total cell number through cytotoxic affects, the percentages of cells containing JCV DNA were normalized to the total cell number on the coverslips (FIG. 36d). CMX001 treatment of 0.01 μM did not result in significant reduction in total cell number, whereas at concentrations of 0.03, 0.07 and 0.1 μM caused 14%, 39% and 46% reductions in total cell number. CDV did not cause cytotoxicity at any concentration tested similar to the results measured by AB staining (FIG. 35b). Normalization of the percentage of JCV DNA containing cells to the total cell number showed that CMX001 caused a dose-dependent reduction in the amount of cells containing JCV DNA, with a maximal suppression of 52% at concentration of 0.1 μM (FIG. 36d). The reduction in JCV DNA containing cells between non-treated and the group of CMX001 treatments tested was significant (p<0.0001). CMX001 reduces JCV DNA replication in SVG cells.

CMX001 is a derivative of CDV which disrupts DNA viruses by inhibiting polymerase function. Therefore, the suppression of JCV multiplication by CMX001 is likely caused by blockage of viral DNA replication by the host DNA polymerase. To determine if JCV DNA replication is affected by CMX001, quantitative real time PCR (qPCR) was used to measure the total viral DNA present in CMX001 treated JCV infected SVG cells. Confluent cultures of SVG cells were exposed to 10 HAU per 5×10⁴ cells of Mad-4 JCV. After overnight JCV exposure, JCV-infected cells were treated with CMX001 or CDV at different concentrations or drug diluent as a non-treated control for 4 days. Total DNA was harvested from the cell cultures and an equal amount of DNA from replicate samples was used in a JCV specific qPCR assay. JCV copy number in the non-treated controls was given a value of 100% and all other values are reported as a percentage of the control. CDV treatment had no affect the copy number of JCV genomes present in SVG cells at any concentration tested. In contrast, CMX001 treatment caused a dose dependant decrease in the number of JCV genome copies present in infected cells by 57% (ρ<0.01) and 60% (ρ<0.01) at concentrations of 0.07 and 0.1 μM respectively. This result demonstrates that CMX001 is affecting JCV DNA replication at some level during an initial infection of SVG cells. The EC₅₀ for CMX001 on JCV infection was 0.045 μM as determined by the concentration of CMX001 that caused a 50% reduction in JCV copy number during infection of SVG cells.

### Limited Cytotoxicity of CMX001 in an Established JCV Infection of SVG Cells.

The results described above demonstrate that CMX001 suppresses JCV multiplication during a new or initial infection of SVG cells. However, by the time that PML is diagnosed in the patient, JCV multiplication in the brain has been occurring for a significant period of time. JCV-infected cells producing high levels of progeny virus will be present at the onset of treatment. Therefore, to determine if CMX001 would be an effective treatment for an ongoing infection we sought to measure the effect of CMX001 on a culture of SVG cells with a previously established infection. To determine the effect of CMX001 on cell viability, non-infected and JCV-infected SVG cells were treated with CMX001 at concentrations ranging from 0.01 to 1 μM for 4 days. Cell viability was measured by MTS analysis. The viability of non-treated cells was given a value of 100% and all other values are provided in reference to the control. Interestingly, CMX001 did not alter cell viability of non-infected or JCV-infected SVG cells at a concentration of 0.01 or 0.1 μM (FIG. 38). However, CMX001 at a concentration of 1 μM caused a 15% reduction in viability of non-infected cells (ρ>0.05) and a 40% reduction in viability of JCV-infected cells (ρ<0.01). This trend is consistent with viability determinations from the initial infections (FIG. 35).

CMX001 Treatment Virtually Eliminates JCV-Infected Cells from an Established Infection.

To determine the effect of CMX001 on JCV multiplication during an ongoing or established infection, non-infected and infected cultures of SVG cells were treated with CMX001 at 0.1 μM or drug diluent for non-treated control for 4 days. CMX001 treatment had minimal effects on cell density or morphology in non-infected or JCV-infected cells as observed by phase contrast microscopy in FIG. 39a, which is consistent with the determination of cell viability in FIG. 38. The presence of JCV DNA in the CMX001 treated cultures was determined by in situ DNA hybridization and cell density was determined by intensity of hematoxylin staining. JCV DNA-containing cells were observed in the non-drug treated infected cultures (FIG. 39b). The percentage of JCV DNA containing cells was quantified and the non-treated control was given a value of 100% and the 0.1 μM treatment was expressed as a percentage of the control. Rare JCV DNA containing cells were present in the CMX001 treated culture (FIG. 39c). CMX001 had a modest affect on cell viability shown in FIG. 39d. The percentages of JCV DNA containing cells were normalized to total cell number (FIG. 39e), demonstrating that CMX001 treatment caused 94% elimination of JCV positive cells from an established infection of SVG cells (p<0.05).

### Discussion

This study demonstrated that the lipid-linked derivative of cidofovir hexadecyloxypropyl-cidofovir, CMX001, suppresses JCV multiplication in the human fetal glial cell line SVG. Cytotoxicity for CMX001 in SVG cells was only observed for concentrations of 1 μM and higher. CMX001 caused a dose dependent decrease of JCV-infected cells during initial infection and significant elimination of JCV-infected cells during an established infection. Quantitative PCR analysis revealed that CMX001 interrupts the ability of JCV to replicate DNA by up to 60%. Suppression of JCV infection at concentrations that are not toxic to the human glial cells and increased bioavailability of the drug in the patient suggests a potential use of CMX001 to limit JCV multiplication in PML patients.

A combination of in situ DNA hybridization and qPCR is a reliable approach to determine JCV DNA replication and virus multiplication. To understand whether CMX001 directly affects JCV replication or indirectly reduces the copy number of JCV by its cytotoxicity all measurements of viral replication were normalized against cell viability. To ensure the accuracy of measuring cell viability different methods were employed in this study, including the MTS assay (FIG. 38), Alamar Blue staining (FIG. 35), and semi-quantification of hematoxylin intensity (FIGS. 36 and 39). All three methods of determining cell viability demonstrated the same trend that CMX001 was only minimally toxic to cells at concentration of 1 μM. Normalization to cell viability clearly demonstrated that CMX001 suppresses JCV infection in the SVG cell model during initial infection (FIG. 36) as well as during an established infection (FIG. 39). These results suggest that CMX001 has the ability to interfere with JCV replication during active infection and could be an appropriate candidate for treatment of PML in the patient.

CMX001 is a derivative of CDV, which has been reported to inhibit viral DNA polymerases from herpes viruses to cytomegalovirus. Quantitative PCR for JCV genome in CMX001 treated JCV-infected SVG cultures demonstrated that CMX001 reduces the level of viral DNA produced by up to 60% (FIG. 37). This result suggests that CMX001 is suppressing viral multiplication at the level of DNA replication. Without viral DNA replication there would not be enough template to produce virion structural proteins as well as DNA to encapsidate, resulting in a severe reduction in the capacity of cells to produce infectious progeny. Because CMX001 has increased bioavailability it is likely that introduction of CMX001 into the brain via the plasma or lymph could significantly reduce virus replication in the brain of PML patients with much greater efficacy of CDV because this drug is more efficient at entering host cells.

CDV at the tested concentration range from 0.01 to 0.1 μM did not show any effect on JCV replication, whereas CMX001 demonstrated a more potent activity in suppressing JCV at these concentrations (FIG. 36). It has been shown that CDV is active only at a concentration range from 20 to 50 μg per ml (63 to 159 μM) in the suppression of Polyomaviruses. JCV multiplication appears to be very sensitive to CMX001 treatment. The effective concentration that produce a 50% of maximal response (EC₅₀) for CMX001 for BK virus infection, another related polyomavirus, has been reported at 0.14 μM. The results described in this study demonstrate that the EC₅₀ of CMX001 for JCV is 0.045 which suggests that CMX001 may be a highly effective drug for the treatment of JCV infection, assuming it can achieve good levels in the CNS. Future studies are required to determine the dose necessary to be effective against JCV multiplication in humans.

This study strongly demonstrates the superior efficacy of CMX001 over CDV as a suppressor of JCV multiplication in a cell culture model. In addition, CMX001 also has many other advantages than CDV, such as oral bioavailability and reduced nephrotoxicity. Based on the efficacies of CMX 001 to reduce JCV infection observed in this study, CMX 001 may be an appropriate drug to evaluate for PML therapy.

TABLE 11
Antiviral agents tested in this study

| Antiviral Agent | Classification | Mechanism of Action | Uses | Structure |
|---|---|---|---|---|
| Cytosine Arabinoside (Ara-C) | Nucleoside Analog | DNA damage, Viral DNA polymerase | Antineoplastic agent, Antiviral-DNA viruses, PML | 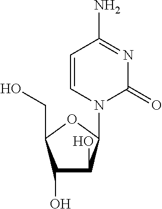 |
| Cidofovir (CDV) | Acyclic nucleoside phosphonate | Viral DNA polymerase | Anti-viral-DNA viruses, PML | 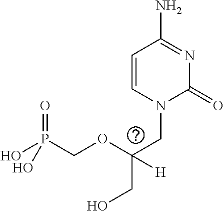 [SMILES: Nc1ccn(CC(CO)OCP(=O)(O)O)c(=O)n1] |
| Hexadecyloxypropyl- Cidofovir (HDP-Cidofovir, CMX001) | Acyclic nucleoside phosphonate | Viral DNA polymerase | Antiviral-DNA viruses | 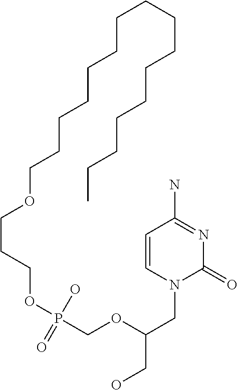 [SMILES: CCCCCCCCCCCCCCCCOCCCOP(=O)(O)COC(CO)Cn1ccc(N)nc1=O] |

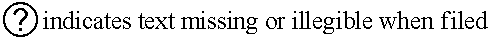

### Example 18

### Enhanced In Vitro Potency of CMX001

Table 12 shows the enhanced in vitro potency of CMX001 versus several dsDNA viruses compared with cidofovir.

TABLE 12

| | | Cidofovir | CMX001 | Enhanced |
|---|---|---|---|---|
| Viral Class | Virus | EC₅₀ (μM) | EC₅₀ (μM) | Activity |
| Adenovirus | AdV 5 | 1.3 | 0.02 | 65 |
| Herpes | HSV 1 | 15 | 0.06 | 250 |
| | HHV 6 | 0.2 | 0.004 | 50 |
| | HCMV | 0.38 | 0.0009 | 422 |
| | EBV | >170 | 0.04 | >4250 |
| Papilloma | HPV 11 | 716 | 17 | 42 |
| Polyoma | BK | 115.1 | 0.13 | 885 |
| | JC | 0.38 | 0.02 | 19 |
| Orthopox | Variola major | 27.3 | 0.1 | 271 |
| | Vaccinia | 46 | 0.8 | 57 |

### Example 19

### CMX001 and Cidofovir-Diphosphate

CMX001 increases cellular exposure to the active antiviral agent, cidofovir-diphosphate (CDV-PP). FIG. 40 shows CMX001 results in 80 times more CDV-PP with 10 times less drug than cidofovir. CDV-PP in PBMCs incubated for 48 hours with CMX001 or cidofovir. The t_{1/2} for CDV-PP was 6.5 days. FIG. 41 shows in vitro intracellular levels of CDV-PP in human PBMCs after incubation with CMX001 for 48 hours. The t_{1/2} for CDV-PP was 3.9 days. FIG. 42 shows in vitro levels of CDV-PP in human PBMCs after incubation with CMX001 for 1 hour. The t_{1/2} for CDV-PP was 6.5 days.

### Example 20

### CMX001 Clearance and Distribution

FIG. 43 shows the clearance of cidofovir or CMX001 from mouse kidney over 4 hours. FIG. 44 shows the organ distribution of CMX001 four hours after an oral dose of 5 mg/kg of [C2-¹⁴C]CMX001. CMX001 is orally available and widely distributed.

### Example 21

### CMX001 Study in Healthy Adult Volunteers

A randomized double-blind, placebo-controlled, single-dose, dose-escalation study of CMX001 in healthy adult volunteers was conducted.

### Single Doses

Nine single dose cohorts- CMX001:placebo (2:1)

Six subjects per cohort; 36 received CMX001; 10 received placebo

Highest dose was 2 mg/kg (140 mg in a 70 kg person)

### Multiple Doses

Six multiple dose cohorts- CMX001:placebo (2:1) (3 doses over 3 weeks)

Five subjects per cohort; 20 received CMX001; 10 received placebo

### Safety

Single and multiple doses were well tolerated; No adverse effects by wireless capsule endoscopy (WCE).

Table 13 shows the human pharmacokinetics after CMX001 2 mg/kg single dose.

TABLE 13

| | | | | |
|---|---|---|---|---|
| | | AUC_{inf}_obs | t_{1/2} | AUCₗₐₛₜ |
| Tₘₐₓ (hr) | Cₘₐₓ (ng/mL) | (hr * ng/mL) | (hr) | (hr * ng/mL) |

CMX001 (plasma):

| | | | | | |
|---|---|---|---|---|---|
| Mean | 3.0 | 350 | 2650 | 24.0 | 2610 |
| SD | | 119 | 445 | 0.7 | 445 |

Cidofovir (plasma):

| | | | | | |
|---|---|---|---|---|---|
| Mean | 11.5 | 31.1 | 1740 | 63.0 | 1510 |
| SD | | 7.0 | 409 | 11.0 | 285 |

### Example 22

### CMX001 Study in Healthy Volunteers

An open-label, 3-way crossover study in healthy volunteers comparing the bioavailability of CMX001 tablet versus solution formulation and the effect of food on CMX001 pharmacokinetics was completed. 24 healthy volunteers received 40 mg tablets (fed), 40 mg tablets (fasted), and 40 mg as a solution (fasted). Ingestion of a high fat meal reduced CMX001 plasma Cₘₐₓ by approximately 40%. The Tₘₐₓ for the tablet was delayed by ˜1 hour compared to that of the liquid formulation in the fasted state. Exposure as indicated by AUC was unchanged. Adverse events were generally mild and there were no serious adverse events.

### Example 23

### CMX001 in Humans with BK Virus

Safety and tolerability of CMX001 in HSCT and renal transplant recipients with BK virus viruria is studied. The safety and tolerability of CMX001 in a post-transplant population is investigated and levels of BKV DNA in urine and plasma over time is monitored.

### Study Design

Twelve HSCT and twelve renal transplant recipients randomized 2:1 (CMX001:Placebo)

BKV viruria >10⁴, renal transplant recipients allowed with viremia <10⁴

Concomitant valgancielovieganciclovir excluded for HSCT recipients only

### Dosing Schedule:

| Treatment | | | | | | |
|---|---|---|---|---|---|---|
| Group | Day 0 | Day 7 | Day 10 | Day 14 | Day 21 | Day 28 |
| CMX001 | 40 mg | 40 mg | 40 mg | 40 mg | 40 mg | 40 mg |
| Placebo | Placebo | Placebo | Placebo | Placebo | Placebo | Placebo |

Table 14 shows safety data (blinded) in renal transplant (RT) subjects (40 mg/wk×5) and Table 15 shows safety data (blinded) in stem cell transplant (SCT) subjects (40 mg/wk×5).

TABLE 14

| | Day 0 | Day 7 | Day 14 | Day 21 | Day 28 |
|---|---|---|---|---|---|
| Serum creatinine | 9 | 9 | 9 | 7 | 5 |
| (mg/dL) number of RT | | | | | |
| subjects | | | | | |
| Mean Serum creatinine | 1.3 | 1.3 | 1.3 | 1.4 | 1.4 |
| (mg/dL) | | | | | |
| Absolute Neutrophil | 8 | 9 | 9 | 7 | 4 |
| Count (1000 per cu mm) | | | | | |
| number of RT subjects | | | | | |
| Mean absolute | 3.16 | 4.88 | 4.59 | 3.46 | 4.42 |
| Neutrophil count (1000 | | | | | |
| per cu mm) | | | | | |
| Alanine | 9 | 9 | 9 | 7 | 6 |
| Aminotransferase (ALT) | | | | | |
| number of RT subjects | | | | | |
| Mean ALT | 19.3 | 22.4 | 21.4 | 19.6 | 18.2 |

TABLE 15

| | Day 0 | Day 7 | Day 14 | Day 21 | Day 28 |
|---|---|---|---|---|---|
| Serum creatinine | 3 | 2 | 2 | 2 | 2 |
| (mg/dL) number of SCT | | | | | |
| subjects | | | | | |
| Mean Serum creatinine | 0.93 | 1.0 | 0.9 | 0.9 | 1.2 |
| (mgldL) | | | | | |
| Absolute Neutrophil | 2 | 1 | 2 | 2 | 2 |
| Count (1000 per cu mm) | | | | | |
| number of SCT subjects | | | | | |
| Mean absolute | 4.40 | 4.40 | 3.55 | 3.50 | 3.15 |
| Neutrophil count (1000 | | | | | |
| per cu mm) | | | | | |
| Alanine | 3 | 1 | 2 | 2 | 2 |
| Aminotransferase (ALT) | | | | | |
| number of SCT subjects | | | | | |
| Mean ALT | 32.7 | 17 | 17.5 | 21.0 | 20.0 |

### Example 24

### CMX001 Phase 2 Study in HSCT Recipients

A multicenter, randomized, double-blind, placebo-controlled, dose-escalation study of the safety, tolerability, and ability of CMX001 to prevent or control cytomegalovirus (CMV) infection in R+ hematopoietic stem cell transplant (HSCT) recipients was designed. The study population is CMV R+ HSCT recipients enrolled 14-30 days post transplant when engrafted. CMX001:Placebo is 2:1. Thirteen subjects enrolled in cohort 1.

### Primary Objectives

Determine the safety and tolerability of CMX001

Determine the ability of CMX001 to prevent or control CMV infection in R+ HSCT recipients.

### Primary Endpoints

Safety Endpoints include clinical assessments and laboratory values, adverse events (and serious adverse events), changes from baseline in laboratory values, vital signs and renal function.

Efficacy Failure Endpoint is CMV DNAemia >200 copies/mL at the conclusion of treatment or diagnosis of CMV disease during the treatment period.

### Example 25

### CMX001 and Progressive Vaccinia

The treatment of a patient with progressive vaccinia was studied; A military recruit, age 20 years, with unknown AML received live virus smallpox vaccine. Post chemotherapy-severe progressive vaccinia, multi-organ failure and pseudomonas sepsis. Poor response to vaccinia immunoglobulin and an investigational antiviral drug-satellite lesions at the vaccination site and lack of antiviral response (as indicated by viral load and serology). The patient was treated with CMX001 (200 mg initial dose) and received increasing doses of other therapies. Within 5 days the virus cultures at the vaccination site became negative for vaccinia virus, and the skin lesions began to heal. The patient received CMX001 every 6 or 7 days for a total of six doses (MMWR 2009; 58:1-4).

Oral ST-246 400 mg was begun on March 5 (51 days post vaccination) and dose increased to 1200 mg on March 25. Oral CMX001 200 mg was given on March 26, then 100 mg weekly for 5 weeks (until April 27). Other treatments included topical ST-246, topical imiquimod and (IV) VIGIV.

### Example 26

### Oral CMX001 Versus IV Cidofovir

FIG. 45 shows a comparison of plasma cidofovir concentrations following IV cidofovir or oral CMX001. Specifically, FIG. 45 shows the estimated cidofovir plasma concentrations in patients given a single intravenous dose of 5 mg/kg cidofovir with probenecid (Cundy, 1999) compared with single dose of 2 mg/kg CMX001 in healthy subjects.

### Example 27

### Antiviral Response to CMX001: Evaluation of First Fifteen EIND Patients

Antiviral activity of CMX001 resulted in all but one of the evaluable patients.

Six AdV patients

5/6 evaluable patients with adenovirus had >99% (2 log 10) reductions in adenovirus or went below the limit of detection. One patient with pre-treatment resistance to cidofovir and CMX001 had approximately a 1.5 log 10 reduction in viremia on CMX001 and a 3 log rebound following discontinuation of CMXOOL

### Six Cytomegalovirus (CMV) Patients

Three were evaluable for viral load reductions and 3/3 responded. Two of the patients who could not be evaluated for reductions in CMV had undetectable levels throughout CMX001 therapy. One patient received only one dose of CMX001 with no virologic follow up.

### Two Polyomavirus BK (BKV) Patients

One had a >1.5 log 10 response.

### One Polyomavirus JC (JCV) Patient

Low levels of JCV in cerebrospinal fluid (CSF) and urine became undetectable on CMX001 therapy.

### One Disseminated Vaccinia Virus (VACV) Patient

Detectable, viable vaceinia in lesions while on ST-246 and vaccinia immune globulin (VIG). Eliminated after treatment with CMX001.

FIG. 46 shows a patient's response of adenovirus viremia to CMX001 treatment. FIG. 47 shows treatment of Epstein-Barr virus (EBV) viremia in a patient with CMX001.

Table 16 shows the response of adenovirus viremia to CMX001 treatment. Table 17 shows the laboratory safety data for 10 patients with high intensity exposure to CMX001 (>19.25 mg/kg/month).

TABLE 16

| | | After 1^{st} | | | |
|---|---|---|---|---|---|
| | | week | After 2^{nd} | | |
| | | Log₁₀ | week Log₁₀ | | |
| | Pre-RX AdV | copies/mL | copies/mL | | |
| | viremia Log₁₀ | (change from | (change from | Associated | |
| Patient | copies/mL | pre-RX) | pre-RX) | infection | Outcome |
| 12 yr | 8.1 | 7.4 (0.7) | 7.0 (1.1) | None | Survived |
| 43 yr | 5.4 | 3.4 (2.0) | 3.3 (2.1) | None | Survived |
| 4 yr | 6.5 | 5.2 (1.3) | 6.2 (0.3) | BKV, HSV | Survived |
| 33 yr | 4.5 | 2.0 (2.5) | 0 (4.5) | None | Survived |
| 20 yr | 7.2 | 3.2 (4.0) | 2.2 (5.0) | BK | Survived |
| 16 yr | 6.4 | 5.4 (1.0) | 6.2 (0.2) | None | Death |
| 66 yr | 5.5 | 5.0 (0.5) | 4.1 (1.3) | BKV, CMV | Death due to |
| | | | | | Graft versus |
| | | | | | Host disease |
| 48 yr | 4.2 | 2.3 (1.9) | 2.2 (2.0) | BKV | Death due to |
| | | | | | Graft versus |
| | | | | | Host disease |

TABLE 17

| | Duration | | | | | | | |
|---|---|---|---|---|---|---|---|---|
| | of | | | Dialysis | | | | |
| | Treatment | Total | | during | | | ANC (1) | ANC (2) |
| No. | (months) | Dose | mg/kg/month | treatment | S Cr (x) | S Cr (y) | WBC (3) | WBC (4) |
| 107923^{a} | 0.73 | 140 | 19.6 | No | 0.3 | 0.1 | 5.2 (3) | 4.3 (4) |
| 107175^{a} | 3.97 | 4320 | 19.8 | Yes | 1.9 | — | 2.17 (3) | — |
| 108085^{a} | 0.5 | 700 | 20.0 | No | 0.9 | 0.7 | 1.1 (1) | 3.3 (2) |
| 108104^{a} | 0.6 | 840 | 20.9 | Yes | 3.2 | 1.3 | — | — |
| 107581^{b} | 2.47 | 2300 | 34.5 | No | 0.4 | 0.7 | — | 7.6 (2) |
| 108106^{b} | 0.87 | 320 | 36.8 | Yes | 0.25 | 0.16 | 3.6 (1) | 3.6 (2) |
| 107836^{c} | 0.83 | 1760 | 38.6 | No | 0.6 | 0.4 | 3.6 (3) | 5.4 (4) |
| 107917^{b} | 1.53 | 280 | 39.8 | No | 0.3 | 0.3 | 1.5 (1) | — |
| 107966^{a} | 0.6 | 240 | 40.0 | No | 0.2 | 0.34 | 3.2 (1) | 5.9 (2) |
| 107878^{b} | 0.1 | 520 | 81.3 | No | 0.7 | 0.7 | 2.4 (1) | 7.7 (2) |

^{a}non-responsive to cidofovir
^{b}no prior cidofovir
^{c}some prior cidofovir
(x), (1), (3)—value on day one of CMX001 treatment
(y), (2), (4)—value on last day of CMX001 treatment

### Example 28

### Treatment of Adenovirus with CMX001

Currently an unmet medical need exists for Adenovirus disease in transplant patients (e.g., HSCT and solid organ transplants (SOT)). HSCT: risk factors include younger age, T-cell depleting regimens, Graft versus host disease (GVHD), others. SOT: typically end-organ disease; may occur later in course; viremia may not be present with disease. For Adenovirus viremia, ≧1000 copies/mL is diagnostic of disease. This is predictive of imminent symptom development, associated with mortality at ≧10,000 copies/mL in plasma. Reduction in viral burden is protective from adenovirus-related mortality.

### Proposed Study

HSCT and SOT patients with adenovirus viremia ≧1000 copies/mL are included. The endpoint is sustained reduction in viremia as measured by drop of 1 log 10 at 28 days. Comparator is second dose of CMX001 (2 mg/kg vs. 4 mg/kg), Ages 9 months and up. Follow-up of 30 days. The study excludes those at risk of imminent demise (includes septic shock). The study allows participation if in renal failure. May discontinue dosing after 3 weeks of sustained undetectability. The study allows for participation in continuation study if: viral load meets endpoint but is not undetectable, or if at risk for rebound of adenovirus disease.

The study does not include patients who have not had a transplant. Analysis is stratified by ALC <300 versus ALC ≧300 cells/mm³ and by SOT versus HSCT. The objective is to show >40% response rate based on historical controls. Secondary endpoints will include reduction in the antiviral AUC and clinical improvement as measured by improvement in a toxicity scale.

### Continuation Study

A continuation study for patients who have completed the proposed study above. CMX001 will be at same dose as in prior study. Participation requires having met the endpoint in the study above and being at ongoing risk from adenovirus disease. Treatment of up to 60 days is allowed. Treatment may be discontinued sooner if adenovirus assays are sustained at undetectable levels for 3 weeks. The objective is control of adenovirus disease.

### Example 29

### Treatment of Cytomegalovirus with CMX001

### Case History

A 46 year-old woman with refractory cytomegalovirus (CMV) and was a D+/R−transplant recipient had a history of recurrent CMV viremia (4 episodes in 3 months). Valganciclovir therapy was complicated by severe neutropenia (3 hospitalizations and ongoing treatment with G-CSF). The patient had a history of renal dysfunction associated with high tacrolimus levels (pre-CMX001 treatment glomerular filtration rate was 51 mL/min.

Treatment with CMX001

Therapy was started with 120 mg CMX001, followed by 60 mg weekly, and dose adjusted to a final dose of 120 mg twice weekly. CMV became undetectable with CMX001 treatment and remained undetectable for the duration of therapy (2 subsequent months). WBC became more robust and G-CSF treatment was decreased.

Treatment with CMX001:

| | | | | | | Week |
|---|---|---|---|---|---|---|
| | Week 1 | Week 2 | Week 5 | Week 10 | Week 11 | 18 |
| CMX001 | 120 mg | 60 mg | 120 mg | 120 mg | 120 mg | 120 mg |
| | qW | qW | | | | |
| Plasma | UD | UD | 3307 | 1541 | UD | UD |
| CMV by | | | | | | |
| PCR | | | | | | |

UD = undetectable

The duration of CMX001 therapy was more than 16 weeks. CMX001 was tolerated without difficulty. Intercurrent events included cough treated with azithromycin, body aches associated with G-CSF, and cholecystectomy for chronic cholecystitis that predated CMX001 therapy. No drug-related adverse changes in renal, liver, or hematologic function were observed.

### Case History

A 50 year-old man with remote kidney transplant (27 years) was treated for refractory cytomegalovirus (CMV). The patient had a kidney transplant in 1981 for glomerulonephritis of unknown etiology. Baseline creatinine was 2.3 mg/dL. The patient developed a fever of unknown origin (FUO) the prior year associated with CMV DNAemia (treated for culture negative endocarditis with 6 weeks of IV antibiotics and for CMV with IV ganciclovir). CMV viremia persisted for many months, with periodic flares, sometimes associated with fevers. Creatinine worsened with ganciclovir/valganciclovir therapy; leflunomide was started but CMV persisted. Renal impairment precluded use of foscarnet or cidofovir. Moderate hypogammaglobulinemia; multiple doses of cytomegalovirus immune globulin (CMVIg) did not help clear viremia. Invasive squamous cell carcinoma status-post finger amputation.

Treatment with CMX001

CMX001 was started at 180 mg followed by 80 mg weekly. Following the first dose of CMX001, CMV plasma DNA became undetectable for the first time in nearly a year. CMX001 was well tolerated. Recurrent squamous cell carcinoma was treated with radiation therapy. CMV DNA rose to 5,037 copies/mL following 2 weeks of skipped doses of CMX001; therapy was reinstated. Creatinine remained stable in the 4.2 to 4.7 range during nearly 3 months of CMX001 therapy. Mycophenolate was discontinued in an attempt at control of the cancer, and kidney rejection ensued. Fatal brain hemorrhage due to increased INR/PT and metastatic cancer occurred; none of the events were considered related to CMX001.

### Case History

A 68 year-old man with double lung transplant and renal failure was treated for refractory cytomegalovirus (CMV). CMV viremia was treated with IV ganciclovir; therapy was switched to foscarnet because of ongoing viremia. Treatment was continued with valganciclovir for prophylaxis once plasma was undetectable for CMV. Reactivation of CMV occurred during a sepsis episode (ganciclovir was started and foscarnet was added after 2 weeks). Dialysis was required for advancing renal failure. Genotype showed A594V resistance mutation to ganciclovir; no UL54 (cidofovir or foscarnet) mutations were detected. Prior ICU care for urinary VRE infection with hypotension and Klebsiella aspiration pneumonia complicated the patient's clinical condition.

Treatment with CMX001

CMV DNA became undetectable following the start of treatment with CMX001. Pharmacokinetic samples were collected following the first, third, and fifth doses, and no dose-adjustment was required based on renal failure. The peak plasma concentration of CMX001 following the first dose was 264 ng/mL and plasma concentrations of cidofovir remained below 250 ng/mL at all observed time points. The patient had multiple ongoing medical problems, but CMV remained suppressed. CMX001 was well tolerated. The patient decided against aggressive care following a massive aspiration. FIG. 48 shows a CMX001 dose and plasma CMV by PCR plot.

### Example 30

### Adenovirus IC₅₀s for CMX001

Table 18 shows several IC₅₀ values for many adenovirus serotypes/isolates.

TABLE 18

| Serotype/Isolate | IC₅₀ (μM) |
|---|---|
| Ad3 ATCC | 0.034 |
| Ad3 | 0.048 |
| Ad4 ATCC | 0.020 |
| Ad4 | 0.032 |
| Ad5 ATCC | 0.046 |
| Ad7 ATCC | 0.065 |
| Ad7 | 0.034 |
| Ad14 ATCC | 0.022 |
| Ad14 | 0.034 |
| Ad14 | 0.041 |
| Ad14 | 0.036 |
| Ad21 | 0.044 |

### Example 31

### CMX001 Prevents Adenovirus-Induced Mortality in an Immunosuppressed Hamster Model

A cyclophosphamide immunosuppressed hamster is infected with adenovirus 5. Viral replication occurs in the liver, adrenals, and pancreas (Toth et al., PNAS 2009). Animals are moribund by day 7.

Upon CMX001 dosing at 2.5 mg/kg/d.i.p. for up to 21 days and dosing pre-challenge or 6 hours, 24 hours or 48 hours, hamsters were rescued from a lethal challenge (˜10⁶ log reduction in liver viremia).

### Example 32

### Effect of CMX001 on HSV-2 Replication in CNS

FIG. 49 shows the effects of CMX001 on Herpes simplex virus-2 (HSV-2) replication in the CNS (Quenelle et al., JID, 2010). The results for CMX001 and acyclovir are reported.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed and that modifications to the disclosed embodiments, as well as other embodiments, are intended to be included within the scope of the appended claims. The invention is defined by the following claims, with equivalents of the claims to be included therein.

1-60. (canceled)

61. A method of treating or ameliorating a viral infection or a viral infection associated disease or disorder in a subject, the method comprising orally administering to the subject a pharmaceutical composition comprising a compound of the following formula: 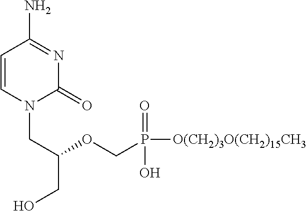
or a pharmaceutically acceptable salt thereof, wherein the compound or the pharmaceutically acceptable salt thereof is administered at a dose of about 1-4 mg/kg once or twice weekly.

62. The method of claim 61, wherein the compound or the pharmaceutically acceptable salt thereof is administered at a dose of 1 mg/kg once weekly.

63. The method of claim 61, wherein the compound or the pharmaceutically acceptable salt thereof is administered at a dose of 2 mg/kg once weekly.

64. The method of claim 61, wherein the compound or the pharmaceutically acceptable salt thereof is administered at a dose of 4 mg/kg once weekly.

65. The method of claim 61, wherein the compound or the pharmaceutically acceptable salt thereof is administered at a dose of 2 mg/kg twice weekly for three weeks, then 3 mg/kg twice weekly.

66. The method of claim 61, wherein the compound or the pharmaceutically acceptable salt thereof is administered at a first dose of 2 mg/kg, a second dose of 1 mg/kg, and then additional doses at 2 mg/kg twice weekly.

67. The method of claim 61, wherein the compound or the pharmaceutically acceptable salt thereof is administered at a first dose of 3 mg/kg, then 2.5 mg/kg twice weekly.

68. The method of claim 61, wherein the compound or the pharmaceutically acceptable salt thereof is administered at a dose of 2 mg/kg twice weekly, then 4 mg/kg twice weekly.

69. The method of claim 61, wherein the compound or the pharmaceutically acceptable salt thereof is administered at a dose of 2 mg/kg twice weekly.

70. The method of claim 61, wherein the compound or the pharmaceutically acceptable salt thereof is administered at a dose of 4 mg/kg twice weekly.

71. The method of claim 61, wherein the viral infection or the viral associated disease or disorder is associated with one or more virus selected from the group consisting of: adenovirus, cytomegalovirus (CMV), JC virus, BK virus, SV40, MCV, KIV, WUV, Epstein-Barr virus (EBV), vaccinia, herpes simplex virus 1, herpes simplex virus 2, human herpes virus 6 (HHV-6), human herpes virus 8 (HHV-8), hepatitis B virus, hepatitis C virus, varicella zoster virus, variola major, variola minor, smallpox, cowpox, camelpox, monkeypox, ebola virus, papilloma virus, human immunodeficiency virus (HIV), influenza, and any combination thereof.

72. A method of treating or ameliorating a viral infection or a viral infection associated disease or disorder in a subject, the method comprising orally administering to the subject a pharmaceutical composition comprising a compound of the following formula: 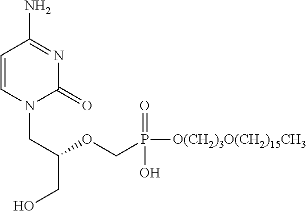
or a pharmaceutically acceptable salt thereof, wherein the compound or the pharmaceutically acceptable salt thereof is administered at a dose of about 40 mg, 50 mg, 70 mg, 75 mg, 100 mg, 150 mg, or 200 mg.

73. The method of claim 72, wherein the compound or the pharmaceutically acceptable salt thereof is administered at a dose of 75 mg twice weekly, 150 mg once weekly, or 150 mg twice weekly.

74. The method of claim 72, wherein the compound or the pharmaceutically acceptable salt thereof is administered at a dose of 100 mg once weekly, 100 mg twice weekly, 200 mg once weekly, or 200 mg twice weekly.

75. The method of claim 72, wherein the compound or the pharmaceutically acceptable salt thereof is administered at a first dose of 200 mg, then 100 mg once weekly.

76. The method of claim 72, wherein the compound or the pharmaceutically acceptable salt thereof is administered at a first dose of 120 mg on first week, then 60 mg on second week, then finally 120 mg twice weekly.

77. The method of claim 72, wherein the compound or the pharmaceutically acceptable salt thereof is administered at a first dose of 180 mg, then 80 mg once weekly.

78. The method of claim 72, wherein the viral infection or the viral associated disease or disorder is associated with one or more virus selected from the group consisting of: adenovirus, cytomegalovirus (CMV), JC virus, BK virus, SV40, MCV, KIV, WUV, Epstein-Barr virus (EBV), vaccinia, herpes simplex virus 1, herpes simplex virus 2, human herpes virus 6 (HHV-6), human herpes virus 8 (HHV-8), hepatitis B virus, hepatitis C virus, varicella zoster virus, variola major, variola minor, smallpox, cowpox, camelpox, monkeypox, ebola virus, papilloma virus, human immunodeficiency virus (HIV), influenza, and any combination thereof.

79. A method of treating or ameliorating a viral infection or a viral infection associated disease or disorder in a subject, the method comprising orally administering to the subject a pharmaceutical composition comprising a therapeutically effective dose of a compound of the following formula: 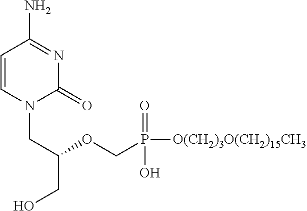
or a pharmaceutically acceptable salt thereof, wherein the pharmaceutical composition comprises one or more antiviral agent selected from the group consisting of: ganciclovir, valganciclovir, foscarnet, acyclovir, valacyclovir, and any combination thereof.

80. The method of claim 79, wherein the viral infection or the viral associated disease or disorder is associated with a virus selected from the group consisting of: adenovirus, herpes virus, cytomegalovirus, and any combination thereof.

81. The method of claim 61, wherein the pharmaceutical composition is administered to a stem cell transplanted subject.

82. The method of claim 72, wherein the pharmaceutical composition is administered to a stem cell transplanted subject.


SEQUENCE LISTING (5 sequences)

SEQ ID NO: 1 - DNA, 24 bp, Artificial Sequence
  Chemically Synthesized Primer Sequence
ggtttccaaggcatactgtgtaac

SEQ ID NO: 2 - DNA, 24 bp, Artificial Sequence
  Chemically Synthesized Primer Sequence
gagaagtgggatgaagacctgttt

SEQ ID NO: 3 - DNA, 24 bp, Artificial Sequence
  Chemically Synthesized Primer Sequence
aggtcttcaaggaactcagcaaga

SEQ ID NO: 4 - DNA, 21 bp, Artificial Sequence
  Chemically Synthesized Primer Sequence
cggcaatcggtttgttgtaaa

SEQ ID NO: 5 - DNA, 20 bp, Artificial Sequence
  Chemically Synthesized DNA probe
ccgtcagccattctctcggc